(19) United States
(12) Patent Application Publication
(10) Pub. No.: US 2012/0129893 A1
(43) Pub. Date: May 24, 2012

(54) Inhibition Of Raf Kinase Using Substituted Heterocyclic Ureas

(76) Inventors: Jacques DUMAS, Orange, CT (US); Uday Khire, Hamden, CT (US); Timothy B. Lowinger, Nishinomiya City (JP); Holger Paulsen, Wuppertal (DE); Bernd Riedl, Wuppertal (DE); William J. Scott, Guilford, CT (US); Roger A. Smith, Madison, CT (US); Jill E. Wood, Hamden, CT (US); Holia Hatoum-Mokdad, Hamden, CT (US); Jeffrey Johnson, Branford, CT (US); Wendy Lee, Hamden, CT (US); Aniko Redman, Derby, CT (US); Robert Sibley, North Haven, CT (US); Joel Renick, San Diego, CA (US)

(21) Appl. No.: 13/349,199

(22) Filed: Jan. 12, 2012

Related U.S. Application Data
(62) Division of application No. 11/768,112, filed on Jun. 25, 2007.
Application No. 11/768,112 is a division of application No. 09/640,780, filed on Aug. 18, 2000, now abandoned.
Application No. 09/640,780 is a continuation of application No. 09/479,125, filed on Dec. 22, 1998, now abandoned.
(60) Provisional application No. 60/135,501, filed on Dec. 22, 1997.

(51) Int. Cl. A61K 31/4439; C07D 401/12; C07D 417/12; A61P 35/02; A61K 31/428; A61K 31/427; A61P 35/00; C07D 231/40; A61K 31/415

(52) U.S. Cl. 514/341; 548/371.7; 546/275.4; 548/171; 548/187; 514/407; 514/367; 514/369

(57) ABSTRACT

Methods of treating tumors mediated by raf kinase, with substituted urea compounds, and such compounds per se.

### FIELD OF THE INVENTION

This invention relates to the use of a group of aryl ureas in treating raf mediated diseases, and pharmaceutical compositions for use in such therapy.

### BACKGROUND OF THE INVENTION

The p21^{ras} oncogene is a major contributor to the development and progression of human solid cancers and is mutated in 30% of all human cancers (Bolton et al. Ann. Rep. Med. Chem. 1994, 29, 165-74; Bos. Cancer Res. 1989, 49, 4682-9). In its normal, unmutated form, the ras protein is a key element of the signal transduction cascade directed by growth factor receptors in almost all tissues (Avruch et al. Trends Biochem, Sci. 1994, 19, 279-83). Biochemically, ras is a guanine nucleotide binding protein, and cycling between a GTP-bound activated and a GDP-bound resting form is strictly controlled by ras' endogenous GTPase activity and other regulatory proteins. In the ras mutants in cancer cells, the endogenous GTPase activity is alleviated and, therefore, the protein delivers constitutive growth signals to downstream effectors such as the enzyme raf kinase. This leads to the cancerous growth of the cells which carry these mutants (Magnuson et al. Semin. Cancer Biol. 1994, 5, 247-53). It has been shown that inhibiting the effect of active ras by inhibiting the raf kinase signaling pathway by administration of deactivating antibodies to raf kinase or by co-expression of dominant negative raf kinase or dominant negative MEK, the substrate of raf kinase, leads to the reversion of transformed cells to the normal growth phenotype (see: Daum et al. Trends Biochem. Sci. 1994, 19, 474-80; Fridman et al. J. Biol. Chem. 1994, 269, 30105-8. Kolch et al. (Nature 1991, 349, 426-28) have further indicated that inhibition of raf expression by antisense RNA blocks cell proliferation in membrane-associated oncogenes. Similarly, inhibition of raf kinase (by antisense oligodeoxynucleotides) has been correlated in vitro and in vivo with inhibition of the growth of a variety of human tumor types (Mania et al., Nat. Med. 1996, 2, 668-75).

### SUMMARY OF THE INVENTION

The present invention provides compounds which are inhibitors of the enzyme raf kinase. Since the enzyme is a downstream effector of p21^{ras}, the instant inhibitors are useful in pharmaceutical compositions for human or veterinary use where inhibition of the raf kinase pathway is indicated, e.g., in the treatment of tumors and/or cancerous cell growth mediated by raf kinase. In particular, the compounds are useful in the treatment of human or animal, e.g., murine cancer, since the progression of these cancers is dependent upon the ras protein signal transduction cascade and therefore susceptible to treatment by interruption of the cascade, i.e., by inhibiting raf kinase. Accordingly, the compounds of the invention are useful in treating solid cancers, such as, for example, carcinomas (e.g., of the lungs, pancreas, thyroid, bladder or colon, myeloid disorders (e.g., myeloid leukemia) or adenomas (e.g., villous colon adenoma).

The present invention therefore provides compounds generally described as aryl ureas, including both aryl and heteroaryl analogues, which inhibit the raf pathway. The invention also provides a method for treating a raf mediated disease state in humans or mammals. Thus, the invention is directed to compounds and methods for the treatment of cancerous cell growth mediated by raf kinase comprising administering a compound of formula I:

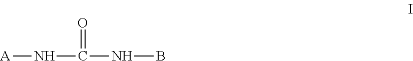

wherein B is generally an unsubstituted or substituted, up to tricyclic, aryl or heteroaryl moiety with up to 30 carbon atoms with at least one 5 or 6 member aromatic structure containing 0-4 members of the group consisting of nitrogen, oxygen and sulfur. A is a heteroaryl moiety discussed in more detail below.

The aryl and heteroaryl moiety of B may contain separate cyclic structures and can include a combination of aryl, heteroaryl and cycloalkyl structures. The substituents for these aryl and heteroaryl moieties can vary widely and include halogen, hydrogen, hydrosulfide, cyano, nitro, amines and various carbon-based moieties, including those which contain one or more of sulfur, nitrogen, oxygen and/or halogen and are discussed more particularly below.

Suitable aryl and heteroaryl moieties for B of formula I include, but are not limited to aromatic ring structures containing 4-30 carbon atoms and 1-3 rings, at least one of which is a 5-6 member aromatic ring. One or more of these rings may have 1-4 carbon atoms replaced by oxygen, nitrogen and/or sulfur atoms.

Examples of suitable aromatic ring structures include phenyl, pyridinyl, naphthyl, pyrimidinyl, benzothiazolyl, quinoline, isoquinoline, phthalimidinyl and combinations thereof, such as, diphenyl ether (phenyloxyphenyl), diphenyl thioether (phenylthiophenyl), diphenylamine (phenylaminophenyl), phenylpyridinyl ether (pyridinyloxyphenyl), pyridinylmethylphenyl, phenylpyridinyl thioether (pyridinylthiophenyl), phenylbenzothiazolyl ether (benzothiazolyloxyphenyl), phenylbenzothiazolyl thioether (benzothiazolylthiophenyl), phenylpyrimidinyl ether, phenylquinoline thioether, phenylhaphthyl ether, pyridinylnapthyl ether, pyridinylnaphthyl thioether, and phthalimidylmethylphenyl.

Examples of suitable heteroaryl groups include, but are not limited to, 5-12 carbon-atom aromatic rings or ring systems containing 1-3 rings, at least one of which is aromatic, in which one or more, e.g., 1-4 carbon atoms in one or more of the rings can be replaced by oxygen, nitrogen or sulfur atoms. Each ring typically has 3-7 atoms. For example, B can be 2- or 3-furyl, 2- or 3-thienyl, 2- or 4-triazinyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-oxadiazol-3- or -5-yl, 1,3,4-thiadiazol-2- or -5-yl, 1,3,4-thiadiazol-3- or -5-yl, 1,2,3-thiadiazol-4- or -5-yl, 2-, 3-, 4-, 5- or 6-2H-thiopyranyl, 2-, 3- or 4-4H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 1-, 3-, 4-, 5-, 6- or 7-benzothiazolyl, 2-, 4-, 5-, 6- or 7-benzisothiazolyl, 2-, 4-, 5-, 6- or 7-benz-1,3-oxadiazolyl, 2-, 3-, 4-, 5-, 6-, 7- or 8-quinolinyl, 1-, 3-, 4-, 5-, 6-, 7-, 8-isoquinolinyl, 1-, 2-, 3-, 4- or 9-carbazolyl, 1-, 2-, 3-, 4-, 5-, 6-, 7-, 8- or 9-acridinyl, or 2-, 4-, 5-, 6-, 7- or 8-quinazolinyl, or additionally optionally substituted phenyl, 2- or 3-thienyl, 1,3,4-thiadiazolyl, 3-pyrryl, 3-pyrazolyl, 2-thiazolyl or 5-thiazolyl, etc. For example, 13 can be 4-methyl-phenyl, 5-methyl-2-thienyl, 4-methyl-2-thienyl, 1-methyl-3-pyrryl, 1-methyl-3-pyrazolyl, 5-methyl-2-thiazolyl or 5-methyl-1,2,4-thiadiazol-2-yl.

Suitable alkyl groups and alkyl portions of groups, e.g., alkoxy, etc., throughout include methyl, ethyl, propyl, butyl, etc., including all straight-chain and branched isomers such as isopropyl, isobutyl, sec-butyl, tert-butyl, etc.

Suitable aryl groups include, for example, phenyl and 1- and 2-naphthyl.

Suitable cycloalkyl groups include cyclopropyl, cyclobutyl, cyclohexyl, etc. The term “cycloalkyl”, as used herein, refers to cyclic structures with or without alkyl substituents such that, for example, “C₄ cycloalkyl” includes methyl substituted cyclopropyl groups as well as cyclobutyl groups. The term “cycloalkyl” also includes saturated heterocyclic groups.

Suitable halogens include F, Cl, Br, and/or I, from one to persubstitution (i.e., all H atoms on the group are replaced by halogen atom), being possible, mixed substitution of halogen atom types also being possible on a given moiety.

As indicated above, these ring systems can be unsubstituted or substituted by substituents such as halogen up to per-halosubstitution. Other suitable substituents for the moieties of B include alkyl, alkoxy, carboxy, cycloalkyl, aryl, heteroaryl, cyano, hydroxy and amine. These other substituents, generally referred to as X and X′ herein, include —CN, —CO₂R⁵, —C(O)NR⁵R^{5′}, —C(O)R⁵, —NO₂, —OR⁵, —SR⁵, —NR⁵R^{5′}, —NR⁵C(O)OR^{5′}, —NR⁵C(O)R^{5′}, C₁-C₁₀ alkyl, C₂-C₁₀ alkenyl, C₁-C₁₀ alkoxy, C₃-C₁₀ cycloalkyl, C₁-C₁₄ aryl, C₇-C₂₄ alkaryl, C₃-C₁₃ heteroaryl, C₄-C₂₃ alkheteroaryl, substituted C₁-C₁₀ alkyl, substituted C₂-C₁₀ alkenyl, substituted C₁-C₁₀ alkoxy, substituted C₃-C₁₀ cycloalkyl, substituted C₄-C₂₃ alkheteroaryl and —Y—Ar.

Where a substituent, X or X′, is a substituted group, it is preferably substituted by one or more substituents independently selected from the group consisting of —CN, —CO₂R⁵, —C(O)R⁵, —C(O)NR⁵R^{5′}, —OR⁵, —NR⁵R^{5′}, —NO₂, —NR⁵C(O)R^{5′}, —NR⁵C(O)OR^{5′} and halogen up to per-halo substitution.

The moieties R⁵ and R^{5′} are preferably independently selected from H, alkyl, alkenyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₂-C₁₀ alkenyl, up to per-halosubstituted C₃-C₁₀ cycloalkyl, up to per-halosubstituted C₆-C₁₄ aryl and up to per-halosubstituted C₃-C₁₃ heteroaryl.

The bridging group Y is preferably —O—, —S—, —N(R⁵)—, —(C₂)—ₘ, —C(O)—, —CH(OH)—, —(CH₂)ₘO—, —(CH₂)ₘS—, —(CH₂)ₘN(R⁵)—, —O(CH₂)ₘ—, —CHX^{a}, —CX^{a}₂, —S—(CH₂)ₘ— and —N(R⁵)(CH₂)ₘ—, where m=1-3, and X^{a} is halogen.

The moiety Ar is preferably a 5-10 member aromatic structure containing 0-4 members of the group consisting of nitrogen, oxygen and sulfur which is unsubstituted or substituted by halogen up to per-halosubstitution and optionally substituted by Zₙ₁, wherein n1 is 0 to 3.

Each Z substituent is preferably independently selected from the group consisting of —CN, —CO₂R⁵, —C(O)NR⁵R^{5′}, —C(O)—NR⁵, —NO₂, —OR⁵, —NR⁵R^{5′}, —NR⁵C(O)OR^{5′}, ═O, —NR⁵C(O)R^{5′}, —SO₂R⁵, —SO₂NR⁵R^{5′}, C₁-C₁₀ alkyl, C₁-C₁₀ alkoxy, C₃-C₁₀ cycloalkyl, C₆-C₁₄ aryl, C₃-C₁₃ heteroaryl, C₇-C₂₄ alkaryl, C₄-C₂₃ alkheteroaryl, substituted C₁-C₁₀ alkyl, substituted C₃-C₁₀ cycloalkyl, substituted C₇-C₂₄ alkaryl and substituted C₄-C₂₃ alkheteroaryl. If Z is a substituted group, it is substituted by the one or more substituents independently selected from the group consisting of —CN, —CO₂R⁵, —C(O)NR⁵R^{5′}, —OR⁵, —SR⁵, —NO₂, —NR⁵R^{5′}, ═O, —NR⁵C(O)R^{5′}, —NR⁵C(O)OR^{5′}, C₁-C₁₀ alkyl, C₁-C₁₀ alkoxy, C₃-C₁₀ cycloalkyl, C₃-C₁₃heteroaryl, C₆-C₁₄ aryl, C₇-C₂₄ alkaryl.

The aryl and heteroaryl moieties of B of Formula I are preferably selected from the group consisting of

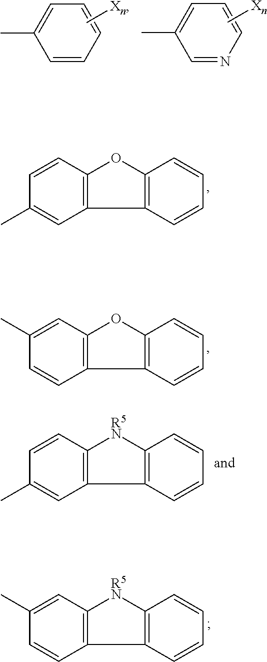

which are unsubstituted or substituted by halogen, up to per-halosubstitution. X is as defined above and n=0-3.

The aryl and heteroaryl moieties of B are more preferably of the formula:

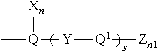

wherein Y is selected from the group consisting of —O—, —S—, —CH₂—, —SCH₂—, —CH₂S—, —CH(OH)—, —C(O)—, —CX^{a}₂, —CX^{a}H—, —CH₂O— and —OCH₂— and X^{a} is halogen.

Q is a six member aromatic structure containing 0-2 nitrogen, substituted or substituted by halogen, up to per-halosubstitution and Q¹ is a mono- or bicyclic aromatic structure of 3 to 10 carbon atoms and 0-4 members of the group consisting of N, O and S, unsubstituted or unsubstituted by halogen up to per-halosubstitution. X, Z, n and n1 are as defined above and s=0 or 1.

In preferred embodiments, Q is phenyl or pyridinyl, substituted or unsubstituted by halogen, up to per-halosubstitution and Q¹ is selected from the group consisting of phenyl, pyridinyl, naphthyl, pyrimidinyl, quinoline, isoquinoline, imidazole and benzothiazolyl, substituted or unsubstituted by halogen, up to per-halo substitution, or Y-Q¹ is phthalimidinyl substituted or unsubstituted by halogen up to per-halo substitution. Z and X are preferably independently selected from the group consisting of —R⁶, —OR⁶, —SR⁶, and —NHR⁷, wherein R⁶ is hydrogen, C₁-C₁₀-alkyl or C₃-C₁₀-cycloalkyl and R⁷ is preferably selected from the group consisting of hydrogen, C₃-C₁₀-alkyl, C₃-C₆-cycloalkyl and C₆-C₁₀-aryl, wherein R⁶ and R⁷ can be substituted by halogen or up to per-halosubstitution.

The heteroaryl moiety A of formula I is preferably selected from the group consisting of:

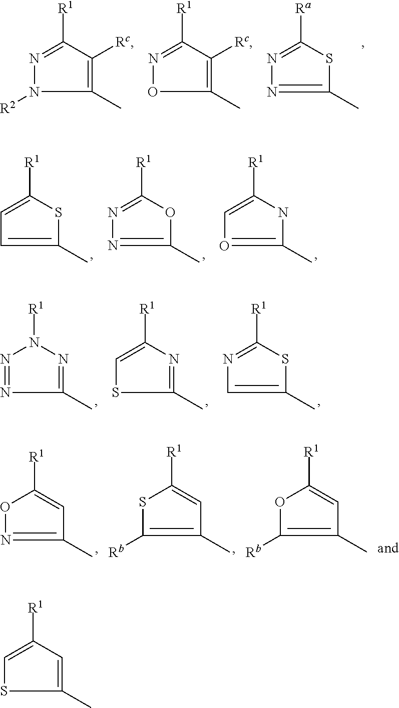

The substituent R¹ is preferably selected from the group consisting of halogen, C₃-C₁₀ alkyl, C₅-C₁₀ cycloalkyl, C₁-C₁₃ heteroaryl, C₆-C₁₃ aryl, C₁-C₂₄ alkaryl, up to per-halosubstituted C₁-C₁₀ alkyl and up to per-halosubstituted C₃-C₁₀ cycloalkyl, up to per-halosubstituted C₁-C₁₀ heteroaryl, up to per-halosubstituted C₆-C₁₃ aryl and up to per-halosubstituted C₁-C₂₄ alkaryl.

The substituent R² is preferably selected from the group consisting of H, —C(O)R⁴, —CO₂R⁴, —C(O)NR³R^{3′}, C₁-C₁₀ alkyl, C₃-C₁₀ cycloalkyl, C₇-C₂₄ alkaryl, C₄-C₂₃ alkheteroaryl, substituted C₁-C₁₀ alkyl, substituted C₃-C₁₀ cycloalkyl, substituted C₇-C₂₄ alkaryl and substituted C₄-C₂₃ alkheteroaryl. Where R² is a substituted group, it is preferably substituted by one or more substituents independently selected from the group consisting of —CN, CO₂R⁴, —C(O)—NR³R^{3′}, —NO₂, —OR⁴, —SR⁴, and halogen up to per-halosubstitution.

R³ and R^{3′} are preferably independently selected from the group consisting of H, SR⁴, —NR⁴R^{4′}, —C(O)₂R⁴, —CO₃R⁴, —C(O)NR⁴R^{4′}, C₁-C₁₀ alkyl, C₃-C₁₀ cycloalkyl, C₆-C₁₄ aryl, C₁-C₁₃ heteroaryl, C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl.

R⁴ and R^{4′} are preferably independently selected from the group consisting of H, C₁-C₁₀ alkyl, C₃-C₁₀ cycloalkyl, C₆-C₁₄ aryl, C₃-C₁₃ heteroaryl; C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl.

R^{a} is preferably C₁-C₁₀ alkyl, C₃-C₁₀ cycloalkyl, up to per-halosubstituted C₁-C₁₀, alkyl and up to per-halosubstituted C₃-C₁₀ cycloalkyl.

R^{b} is preferably hydrogen or halogen.

R^{c} is hydrogen, halogen, C₁-C₁₀ alkyl, up to per-halosubstituted C₁-C₁₀ alkyl or combines with R¹ and the ring carbon atoms to which R¹ and R^{c} are bound to form a 5- or 6-membered cycloalkyl, aryl or hetaryl ring with 0-2 members selected from O, N and S;

The invention also relates to compounds of general formula I described above and includes pyrazoles, isoxazoles, thiophenes, furans and thiadiazoles. These more particularly include pyrazolyl ureas of the formula

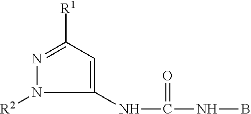

wherein R², R¹ and B are as defined above;
and both 5,3- and 3,5-isoxazolyl ureas of the formulae

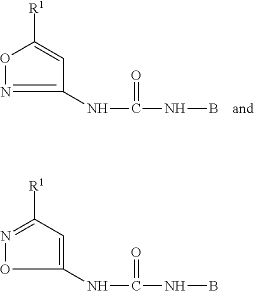

wherein R¹ and B are also as defined above.

Component B for these compounds is a 1-3 ring aromatic ring structure selected from the group consisting of:

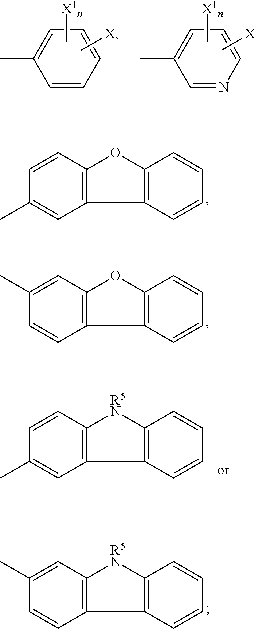

which is substituted or unsubstituted by halogen, up to per-halosubstitution. Here R⁵ and R^{5′} are as defined above, n=0-2 and each X¹ substituent is independently selected from the group of X or from the group consisting of —CN, —CO₂R⁵, —C(O)R⁵, —C(O)NR⁵R^{5′}, —OR⁵, —NO₂, —NR⁵R^{5′}, C₁-C₁₀ alkyl, C₂₋₁₀-alkenyl, C₁₋₁₀-alkoxy, C₃-C₁₀ cycloalkyl, C₆-C₁₄ aryl and C₇-C₂₄ alkaryl. The substituent X is selected from the group consisting of —SR⁵, —NR⁵C(O)OR^{5′}, NR⁵C(O)R^{5′}, C₃-C₁₃ heteroaryl, C₄-C₂₃ alkheteroaryl, substituted C₁-C₁₀ alkyl, substituted C₂₋₁₀-alkenyl, substituted C₁₋₁₀-alkoxy, substituted C₃-C₁₀ cycloalkyl, substituted C₆-C₁₄ aryl, substituted C₇-C₁₄ alkaryl, substituted C₃-C₁₃ heteroaryl, substituted C₄-C₂₃ alkheteroaryl, and —Y—Ar, where Y and Ar are as defined above. If X is a substituted group, as indicated previously above, it is substituted by one or more substituents independently selected from the group consisting of —CN, —CO₂R⁵, —C(O)R⁵, —C(O)NR⁵R^{5′}, —OR⁵, —SR⁵, —NR⁵R^{5′}, NO₂, —NR⁵C(O)R^{5′}, —NR⁵C(O)OR^{5′} and halogen up to per-halosubstitution, where R⁵ and R^{5′} are as defined above.

The components of B are subject to the following provisos, where R¹ is t-butyl and R² is methyl for the pyrazolyl ureas, B is not

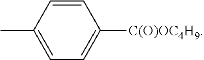

Where R¹ is t-butyl for the 5,3-isoxazolyl ureas, B is not

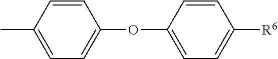

wherein R⁶ is —NHC(O)—O-t-butyl, —O-n-pentyl, —O-n-butyl, —O-propyl, —C(O)NH—(CH₃)₂, —OCH₂CH(CH₃)₂, or —O—CH₂-phenyl. Where R¹ is t-butyl for the 3,5-isoxazole ureas, B is not

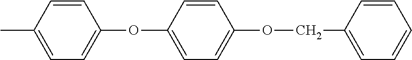

and where R¹ is —CH, -t-butyl for the 3,5-isoxazolyl ureas, B is not

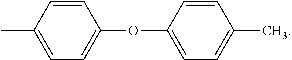

Preferred pyrazolyl ureas, 3,5-isoxazolyl ureas and 5,3-isoxazolyl ureas are those wherein B is of the formula

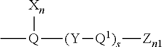

wherein, Q, Q¹, X, Z, Y, n, s and n1 are as defined above.

Preferred pyrazole ureas more particularly include those wherein Q is phenyl or pyridinyl, Q′ is pyridinyl, phenyl or benzothiazolyl, Y is —O—, —S—, —CH₂S—, —SCH₂—, —CH₂O—, —OCH₂— or —CH₂—, and Z is H, —SCH₃, or —NH—C(O)—CₚH₂ₚ₊₁, wherein p is 1-4, n=0, s=1 and n1=0-1. Specific examples of preferred pyrazolyl ureas are:
N-(3-tert-Butyl-5-pyrazolyl)-N′-(4-phenyloxyphenyl)urea;
N-(3-tert-Butyl-5-pyrazolyl)-N′-(3-(3-methylaminocarbonylphenyl)-oxyphenyl)urea;
N-(3-tert-Butyl-5-pyrazolyl)-N′-(3-(4-pyridinyl)thiophenyl)urea;
N-(3-tert-Butyl-5-pyrazolyl)-N′-(4-(4-pyridinyl)thiophenyl)urea;
N-(3-tert-Butyl-5-pyrazolyl)-N′-(4-(4-pyridinyl)oxyphenyl)urea;
N-(3-tert-Butyl-5-pyrazolyl)-N′-(4-(4-pyridinyl)methylphenyl)urea;
N-(1-Methyl-3-tert-butyl-5-pyrazolyl)-N′-(4-phenyloxyphenyl)urea;
N-(1-Methyl-3-tert-butyl-5-pyrazolyl)-N′-(3-(4-pyridinyl)thiophenyl)urea;
N-(1-Methyl-3-tert-butyl-5-pyrazolyl)-N′-((4-(4-pyridinyl)thiomethyl)-phenyl)urea;
N-(1-Methyl-3-tert-butyl-5-pyrazolyl)-N′-(4-(4-pyridinyl)thiophenyl)urea;
N-(1-Methyl-3-tert-butyl-5-pyrazolyl)-N′-(4-(4-pyridinyl)oxyphenyl)urea;
N-(1-Methyl-3-tert-butyl-5-pyrazolyl)-N′44-(4-pyridinyl)methyloxy)phenyl)-urea;
N-(1-Methyl-3-tert-butyl-5-pyrazolyl)-N′-(3-(2-benzothiazolyl)oxyphenyl)-urea;
N-(3-tert-butyl-5-pyrazolyl)-N′-(3-(4-pyridyl)thiophenyl)urea;
N-(3-tert-butyl-5-pyrazolyl)-N′-(4-(4-pyridyl)thiophenyl)urea;
N-(3-tert-butyl-5-pyrazolyl)-N′-(3-(4-pyridyl)oxyphenyl)urea;
N-(3-tert-butyl-5-pyrazolyl)-N′-(4-(4-pyridyl)oxyphenyl)urea;
N-(1-methyl-3-tert-butyl-5-pyrazolyl)-N′-(3-(4-pyridyl)thiophenyl)urea;
N-(1-methyl-3-tert-butyl-5-pyr azolyl)-Nfr-(4-(4-pyridyl)thiophenyl)urea;
N-(1-methyl-3-tert-butyl-5-pyrazolyl)-N′-(3-(4-pyridyl)oxyphenyl)urea; and
N-(1-methyl-3-tert-butyl-5-pyrazolyl)-N′-(4-(4-pyridyl)oxyphenyl)urea.

Preferred 3,5-isoxazolyl ureas more particularly include those wherein Q is phenyl or pyridinyl, Q¹ is phenyl, benzothiazolyl or pyridinyl, Y is —O—, —S— or —CH₂—, Z is —CH₃, Cl, —OCH₃ or —C(O)—CH₃, n=0, s=1, and n1=0-1. Specific examples of preferred 3,5-isoxazolyl ureas are:
N-(3-Isopropyl-5-isoxazolyl)-N′-(4-(4-pyridinyl)thiophenyl)urea;
N-(3-tert-Butyl-5-isoxazolyl)-N′-(4-(4-methoxyphenyl)oxyphenyl)urea;
N-(3-tert-Butyl-5-isoxazolyl)-N′-(5-(2-(4-acetylphenyl)oxy)pyridinyl)urea;
N-(3-tert-Butyl-5-isoxazolyl)-N′-(3-(4-pyridinyl)thiophenyl)urea;
N-(3-wt-Butyl-5-isoxazolyl)-N′-(4-(4-pyridinyl)methylphenyl)urea;
N-(3-tert-Butyl-5-isoxazolyl)-N′-(4-(4-pyridinyl)thiophenyl)urea;
N-(3-tert-Butyl-5-isoxazolyl)-N′-(4-(4-pyridinyl)oxyphenyl)urea;
N-(3-tert-Butyl-5-isoxazolyl)-N)-(4-(4-methyl-3-pyridinyl)oxyphenyl)urea;
N-(3-tert-Butyl-5-isoxazolyl)-N′-(3-(2-benzothiazolyl)oxyphenyl)urea;
N-(3-(1,1-Dimethylpropyl)-5-isoxazolyl)-N)-(4-(4-methylphenyl)oxyphenyl)-urea;
N-(3-(1,1-Dimethylpropyl)-5-isoxazolyl)-N′-(3-(4-pyridinyl)thiophenyl)urea;
N-(3-(1,1-Dimethylpropyl)-5-isoxazolyl)-N′-(4-(4-pyridinyl)oxyphenyl)urea;
N-(3-(1,1-Dimethylpropyl)-5-isoxazolyl)-N′-(4-(4-pyridinyl)thiophenyl)urea;
N-(3-(1,1-Dimethylpropyl-5-isoxazolyl)-N′-(5-(2-(4-methoxyphenyl)oxy)-pyridinyl)urea;
N-(3-(1-Methyl-1-ethylpropyl)-5-isoxazolyl)-N′-(4-(4-pyridinyl)oxyphenyl)-urea;
N-(3-(1-Methyl-1-ethylpropyl)-5-isoxazolyl)-N′-(3-(4-pyridinyl)thiophenyl)-urea;
N-(3-isopropyl-5-isoxazolyl)-N′-(3-(4-(2-methylcarbamoyl)pyridyl)-oxyphenyl)urea;
N-(3-isopropyl-5-isoxazolyl)-N′-(4-(4-(2-methylcarbamoyl)pyridyl)-oxyphenyl)urea;
N-(3-tert-butyl-5-isoxazolyl)-N′-(3-(4-(2-methylcarbamoyl)-pyridyl)oxyphenyl)urea;
N-(3-tert-butyl-5-isoxazolyl)-N′-(4-(4-(2-methylcarbamoyl)pyridyl)-oxyphenyl)urea;
N-(3-tert-butyl-5-isoxazolyl)-N′-(3-(4-(2-methylcarbamoyl)pyridyl)-thiophenyl)urea;
N-(3-(1,1-dimethylprop-1-yl)-5-isoxazolyl)-N′-(3-(4-(2-methylcarbamoyl)-pyridyl)oxyphenyl)urea;
N-(3-(1,1-dimethylprop-1-yl)-5-isoxazolyl)-N′-(4-(4-(2-methylcarbamoyl)-pyridyl)oxyphenyl)urea; and
N-(3-tert-butyl-5-isoxazolyl)-N′-(3-chloro-4-(4-(2-methylcarbamoyl)pyridyl)-thiophenyl)urea.

Preferred 5,3-isoxazolyl ureas more particularly include those wherein Q is phenyl or pyridinyl, Q′ is phenyl, benzothiazolyl or pyridinyl, Y is —O—, —S— or —CH₂—, X is CH₃ and Z is —C(O)NH—, CₚH₂ₚ₊₁, wherein p=1-4, —C(O)CH₃, —CH₃, —OH, —OC₂H₅, —CN, phenyl, or —OCH₃, n=0 or 1, s=0 or 1, and n1=0 or 1. Specific examples of preferred 5,3-isoxazolyl ureas are:
N-(5-tert-Butyl-3-isoxazolyl)-N′-(4-(4-hydroxyphenyl)oxyphenyl)urea;
N-(5-tert-Butyl-3-isoxazolyl)-N′44-(3-hydroxyphenyl)oxyphenyl)urea;
N-(5-tert-Butyl-3-isoxazolyl)-N′-(4-(4-acetylphenyl)oxyphenyl)urea;
N-(5-tert-Butyl-3-isoxazolyl)-N′-(3-benzoylphenyl)urea;
N-(5-tert-Butyl-3-isoxazolyl)-N′-(4-phenyloxyphenyl)urea;
N-(5-tert-Butyl-3-isoxazolyl)-N′-(4-(3-methylaminocarbonylphenyl)-thiophenyl)urea;
N-(5-tert-Butyl-3-isoxazolyl)-N′-(4-(4-(1,2-methylenedioxy)phenyl)-oxyphenyl)urea;
N-(5-tert-Butyl-3-isoxazolyl)-N′-(4-(3-pyridinyl)oxyphenyl)urea;
N-(5-tert-Butyl-3-isoxazolyl)-N′-(4-(4-pyridinyl)oxyphenyl)urea;
N-(5-tert-Butyl-3-isoxazolyl)-N′-(4-(4-pyridyl)thiophenyl)urea;
N-(5-tert-Butyl-3-isoxazolyl)-N′-(4-(4-pyridinyl)methylphenyl)urea;
N-(5-tert-Butyl-3-isoxazolyl)-N′-(3-(4-pyridinyl)oxyphenyl)urea;
N-(5-tert-Butyl-3-isoxazolyl)-N′-(3-(4-pyridinyl)thiophenyl)urea;
N-(5-tert-Butyl-3-isoxazolyl)-N′-(3-(3-methyl-4-pyridinyl)oxyphenyl)urea;
N-(5-tert-Butyl-3-isoxazolyl)-N′-(3-(3-methyl-4-pyridinyl)thiophenyl)urea;
N-(5-tert-Butyl-3-isoxazolyl)-N′-(4-(3-methyl-4-pyridinyl)thiophenyl)urea;
N-(5-tert-Butyl-3-isoxazolyl)-N′-(3-(4-methyl-3-pyridinyl)oxyphenyl)urea;
N-(5-tert-Butyl-3-isoxazolyl)-N′-(4-(3-methyl-4-pyridinyl)oxyphenyl)urea;
N-(5-tert-Butyl-3-isoxazolyl)-N′-(3-(2-benzothiazolyl)oxyphenyl)urea;
N-(5-tert-butyl-3-isoxazolyl)-N′-(3-chloro-4-(4-(2-methylcarbamoyl)pyridyl)-oxyphenyl)urea;
N-(5-tert-butyl-3-isoxazolyl)-N′-(4-(4-(2-methylcarbamoyl)pyridyl)-oxyphenyl)urea;
N-(5-tert-butyl-3-isoxazolyl)-N′-(3-(4-(2-methylcarbamoyl)pyridyl)-thiophenyl)urea;
N-(5-tert-butyl-3-isoxazolyl)-N′-(2-methyl-4-(4-(2-methylcarbamoyl)pyridyl)-oxyphenyl)urea;
N-(5-tert-butyl-3-isoxazolyl)-N′-(4-(4-(2-carbamoyl)pyridyl)oxyphenyl)urea;
N-(5-tert-butyl-3-isoxazolyl)-N′-(3-(4-(2-carbamoyl)pyridyl)oxyphenyl)urea;
N-(5-tert-butyl-3-isoxazolyl)-N′-(3-(4-(2-methylcarbatnoyppyridyl)-oxyphenyl)urea;
N-(5-tert-butyl-3-isoxazolyl)-N′-(4-(4-(2-methylcarbamoyl)pyridyl)-thiophenyl)urea;
N-(5-tert-butyl-3-isoxazolyl)-N′-(3-chloro-4-(4-(2-methylcarbamoyl)pyridyl)-oxyphenyl)urea; and
N-(5-tert-butyl-3-isoxazolyl)-N′-(4-(3-methylcarbamoyl)phenyl)oxyphenyl)urea.

Additionally included are thienyl ureas of the formulae

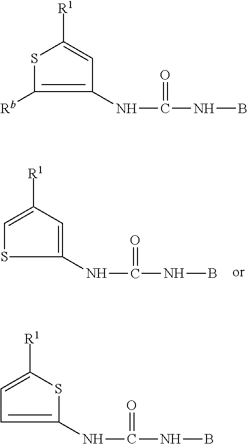

wherein R¹, R^{b} and B are as defined above. Preferred B components for the thienyl ureas of this invention have aromatic ring structures selected from the group consisting of:

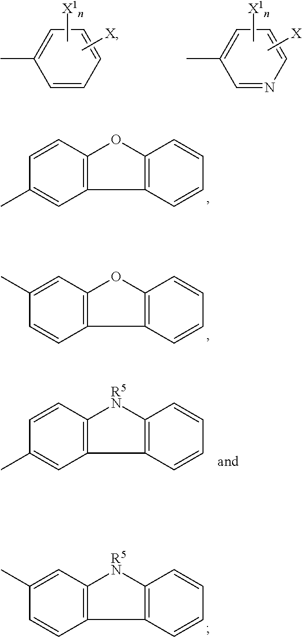

These aromatic ring structures can be substituted or unsubstituted by halogen, up to per-halosubstitution. The X¹ substituents are independently selected from the group consisting of X or from the group consisting of, —CN, —OR⁵, —NR⁵R^{5′}, C₁-C₁₀ alkyl. The X substituents are independently selected from the group consisting of —CO₂R⁵, —C(O)R⁵R^{5′}, —C(O)R⁵, —NO₂, —SR⁵, —NR⁵C(O)OR^{5′}, —NR⁵C(O)R⁵′, C₃-C₁₀ cycloalkyl, C₆-C₁₄ aryl, C₇-C₂₄ alkaryl, C₃-C₁₃ heteroaryl, C₄-C₂₃ alkheteroaryl, and substituted C₁-C₁₀ alkyl, substituted C₂-C₁₀-alkenyl, substituted C₁₋₁₀-alkoxy, substituted C₃-C₁₀ cycloalkyl, substituted C₆-C₁₄ aryl, substituted C₇-C₂₄ alkaryl, substituted C₃-C₁₃ heteroaryl, substituted C₄-C₂₃ alkheteroaryl, and —Y—Ar. Where X is a substituted group, it is substituted by one or more substituents independently selected from the group consisting of —CN, —CO₂R⁵, —C(O)R⁵, —C(O)NR⁵R^{5′}, —OR⁵, —SR⁵, —NR⁵R^{5′}, —NO₂, —NR⁵C(O)R^{5′}, —NR⁵C(O)OR⁵R^{5′} and halogen up to per-halo substitution. The moieties R⁵, R^{5′}, Y and Ar are as defined above and n=0-2.

The components for B are subject to the proviso that where R¹ is t-butyl and R^{b} is H for the 3-thienyl ureas, B is not of the formula

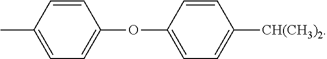

Preferred thienyl ureas include those wherein B is of the formula

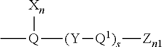

and Q, Q¹, Y, X, Z, n, s and n1 are as defined above. The preferred thienyl ureas more particularly include those wherein Q is phenyl, Q¹ is phenyl or pyridinyl, Y is —O— or —S—, Z is —Cl, —CH₃, —OH or —OCH₃, n=0, s=0 or 1, and n1=0-2. Specific examples of preferred thienyl ureas are:
N-(3-Isopropyl-5-isoxazolyl)-N′-(4-(4-pyridinyl)thiophenyl)urea;
N-(3-tert-Butyl-5-isoxazolyl)-N′-(4-(4-methoxyphenyl)oxyphenyl)urea;
N-(3-tert-Butyl-5-isoxazolyl)-N′-(5-(2-(4-acetylphenyl)oxy)pyridinyl)urea;
N-(3-tert-Butyl-5-isoxazolyl)-N′-(3-(4-pyridinyl)thiophenyl)urea;
N-(3-tert-Butyl-5-isoxazolyl)-N′-(4-(4-pyridinyl)methylphenyl)urea;
N-(3-tert-Butyl-5-isoxazolyl)-N′-(4-(4-pyridinyl)thiophenyl)urea;
N-(3-tert-Butyl-5-isoxazolyl)-N′-(4-(4-pyridinyl)oxyphenyl)mea;
N-(3-tert-Butyl-5-isoxazolyl)-N′-(4-(4-methyl-3-pyridinyl)oxyphenyl)urea;
N-(3-tert-Butyl-5-isoxazolyl)-N′-(3-(2-benzothiazolyl)oxyphenyl)urea;
N-(3-(1,1-Dimethylpropyl)-5-isoxazolyl)-N′-(4-(4-methylphenyl)-oxyphenyl)urea;
N-(3-(1,1-Dimethylpropyl)-5-isoxazolyl)-N′-(3-(4-pyridinyl)thiophenyl)urea;
N-(3-(1,1-Dimethylpropyl)-5-isoxazolyl)-N′-(4-(4-pyridinyl)oxyphenyl)urea;
N-(3-(1,1-Dimethylpropyl)-5-isoxazolyl)-N′-(4-(4-pyridinyl)thiophenyl)urea;
N-(3-(1,1-Dimethylpropyl-5-isoxazolyl)-N′-(5-(2-(4-methoxyphenyl)-oxy)pyridinyl)urea;
N-(3-(1-Methyl-1-ethylpropyl)-5-isoxazolyl)-N′-(4-(4-pyridinyl)-oxyphenyl)urea; and
N-(3-(1-Methyl-1-ethylpropyl)-5-isoxazolyl)-N′-(3-(4-pyridinyl)thio-phenyl)urea.

Preferred thiophenes include:
N-(5-tert-butyl-3-thienyl)-N′-(4-(4-methoxyphenyl)oxyphenyl)urea;
N-(5-tert-butyl-3-thienyl)-N′-(4-(4-hydroxyphenyl)oxyphenyl)urea;
N-(5-tert-butyl-3-thienyl)-N′-(4-(3-methylphenyl)oxyphenyl)urea; and
N-(5-tort-butyl-3-thienyl)-N′-(4-(4-pyridyl)thiophenyl)urea; and

Also included are the thiadiazolyl and furyl ureas of the formulae:

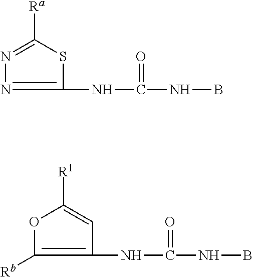

wherein R^{a}, R^{b}, R¹ and B are as defined above. The thiadiazolyl and furyl ureas have preferred aromatic ring structures for B identical to those for the pyrazolyl, thienyl and isoxazolyl ureas shown above. Such ring structures can be unsubstituted or substituted by halogen, up to per-halosubstitution, and each X¹ substituent is independently selected from the group consisting of X or from the group consisting of —CN, —NO₂, —OR⁵ and C₁-C₁₀ alkyl. The X substituents are selected from the group consisting of —SR⁵, —CO₂R⁵, —C(O)R⁵, —C(O)NR⁵R^{5′}, —NR⁵R^{5′}, —NR⁵C(O)OR^{5′}, —NR⁵C(O)R^{5′}, substituted C₂₋₁₀-alkenyl, substituted C₁₋₁₀-alkoxy, C₃-C₁₀ cycloalkyl, —C₆-C₁₄ aryl, —C₇-C₂₄, alkaryl, C₃-C₁₃ heteroaryl, C₄-C₂₃ alkheteroaryl, and substituted C₁-C₁₀ alkyl, substituted C₃-C₁₀ cycloalkyl, substituted aryl, substituted alkaryl, substituted heteroaryl, substituted C₄-C₂, alkheteroaryl and —Y—Ar. Each of R⁵, R^{5′} and Ar are as defined above, n=0-2, and the substituents on X where X is a substituted group are as defined for the pyrazolyl, isoxazolyl and thienyl ureas.

This invention also includes pharmaceutical compositions that include compounds described above and a physiologically acceptable carrier.

Preferred faryl ureas and thiadiazole ureas include those wherein B is of the formula

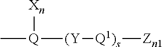

and Q, Q¹, X, Y, Z, n, s, and n1 are as defined above. The preferred thiadaizolyl ureas more particularly include those wherein Q is phenyl, Q¹ is phenyl or pyridinyl, Y is —O— or —S—, n=0, s=1 and n1=0. Specific examples of preferred thiadiazolyl ureas are:
N-(5-tert-Butyl-2-(1-thia-3,4-diazolyl))-N′-(3-(4-pyridinyl)thiophenyl)urea;
N-(5-tert-Butyl-2-(1-thia-3,4-diazolyl))-N′-(4-(4-pyridinyl)oxyphenyl)urea;
N-(5-tert-butyl-2-(1-thia-3,4-diazolyl))-N′-(3-(4-(2-methylcarbamoyl)pyridyl)-oxyphenyl)urea;
N-(5-tert-butyl-2-(1-thia-3,4-diazolyl))-N′-(4-(4-(2-methylcarbamoyl)pyridyl)-oxyphenyl)urea;
N-(5-tert-butyl-2-(1-thia-3,4-diazolyl))-N′-(3-chloro-4-(4-(2-methylcarbamoyl)pyridyl)oxyphenyl)urea;
N-(5-tert-butyl-2-(1-thia-3,4-diazolyl))-N′-(2-chloro-4-(4-(2-methylcarbamoyl)pyridyl)oxyphenyl)urea;
N-(5-tert-butyl-2-(1-thia-3,4-diazolyl))-N′-(3-(4-pyridyl)thiophenyl)urea;
N-(5-tert-butyl-2-(1-thia-3,4-diazolyl))-N′-(2-methyl-4-(4-(2-methylcarbamoyl)pyridyl)oxyphenyl)urea; and
N-(5-(1,1-dimethylprop-1-yl)-2-(1 carbamoylphenyl)oxyphenyl)urea.

The preferred furyl ureas more particularly include those wherein Q is phenyl, Q¹ is phenyl or pyridinyl, Y is —O— or —S—, Z is —Cl or —OCH, s=0 or 1, n=0 and n1=0-2.

The present invention is also directed to pharmaceutically acceptable salts of formula I. Suitable pharmaceutically acceptable salts are well known to those skilled in the art and include basic salts of inorganic and organic acids, such as hydrochloric acid, hydrobromic acid, sulphuric acid, phosphoric acid, methanesulphonic acid, sulphonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid. In addition, pharmaceutically acceptable salts include acid salts of inorganic bases, such as salts containing alkaline cations (e.g., Li⁺ Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations such as those arising from protonation or peralkylation of triethylamine, N,N-diethylamine, N,N-dicyclohexylamine, pyridine, N,N-dimethylaminopyridine (DMAP), 1,4-diazabiclo[2.2.2]octane (DABCO), 1,5-diazabicyclo[4.3.0]non-5-ene (DBN) and 1,8-diazabicyclo[5.4.0]undec-7-ene (DBU).

A number of the compounds of Formula I possess asymmetric carbons and can therefore exist in racemic and optically active forms. Methods of separation of enantiomeric and diastereomeric mixtures are well known to one skilled in the art.

The present invention encompasses any isolated racemic or optically active form of compounds described in Formula I which possess Raf kinase inhibitory activity.

### General Preparative Methods

The compounds of Formula I may be prepared by use of known chemical reactions and procedures, some of which are commercially available. Nevertheless, the following general preparative methods are presented to aid one of skill in the art in synthesizing the inhibitors, with more detailed examples being presented in the experimental section describing the working examples.

Heterocyclic amines may be synthesized utilizing known methodology (Katritzky, et al. Comprehensive Heterocyclic Chemistry; Permagon Press: Oxford, UK (1984). March. Advanced Organic Chemistry, 3^{rd} Ed.; John Wiley: New York (1985)). For example, 3-substituted-5-aminoisoxazoles (3) are available by the reaction of hydroxylamine with an α-cyanoketone (2), as shown in Scheme 1. Cyanoketone 2, in turn, is available from the reaction of acetamidate ion with an appropriate acyl derivative, such as an ester, an acid halide, or an acid anhydride. Reaction of an cyanoketone with hydrazine (R²=H) or a monosubstituted hydrazine affords the 3-substituted- or 1,3-disubstituted-5-aminopyrazole (5). Pyrazoles unsubstituted at N-1 (R²=H) may be acylated at N-1, for example using di-tert-butyl dicarbonate, to give pyrazole 7. Similarly, reaction of nitrile 8 with an -thioacetatc ester gives the 5-substituted-3-amino-2-thiophenecarboxylate (9, Ishizaki et al. JP 6025221). Decarboxylation of ester 9 may be achieved by protection of the amine, for example as the tert-butoxy (BOC) carbamate (10), followed by saponification and treatment with acid. When BOC protection is used, decarboxylation may be accompanied by deprotection giving the substituted 3-thiopheneammonium salt 11. Alternatively, ammonium salt 11 may be directly generated through saponification of ester 9 followed by treatment with acid.

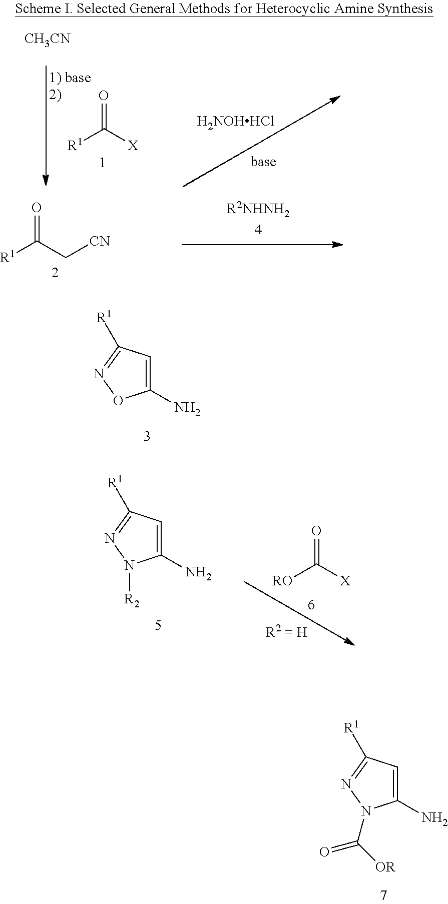

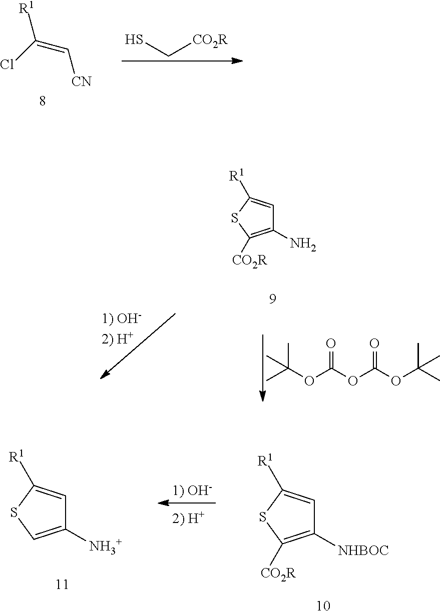

Substituted anilines may be generated using standard methods (March. Advanced Organic Chemistry, 3^{rd} Ed.; John Wiley: New York (1985); Larock. Comprehensive Organic Transformations; VCH Publishers: New York (1989)). As shown in Scheme II, aryl amines are commonly synthesized by reduction of nitroaryls using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroaryls may also be directly reduced using a strong hydride source, such as Li (Seyden-Penne. Reductions by the Alumino- and Borohydrides in Organic Synthesis; VCH Publishers: New York (1991)), or using a zero valent metal, such as Fe, Sn or Ca, often in acidic media. Many methods exist for the synthesis of nitroaryls (March. Advanced Organic Chemistry, 3^{rd} Ed.; John Wiley: New York (1985). Larock. Comprehensive Organic Transformations; VCH Publishers: New York (1989)).

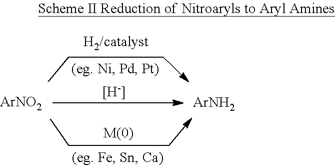

Nitroaryls are commonly formed by electrophilic aromatic nitration using HNO₃, or an alternative NO₂⁺ source. Nitroaryls may be further elaborated prior to reduction. Thus, nitro aryls substituted with

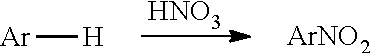

potential leaving groups (eg. F, Cl, Br, etc.) may undergo substitution reactions on treatment with nucleophiles, such as thiolate (exemplified in Scheme III) or phenoxide. Nitroaryls may also undergo Ullman-type coupling reactions (Scheme III).

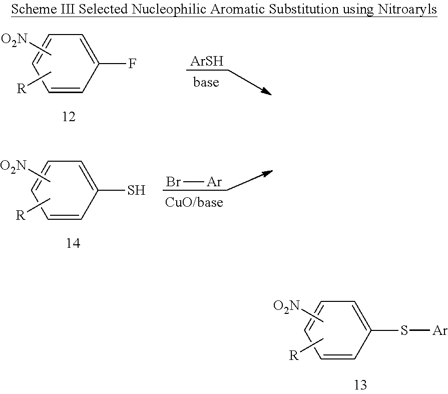

As shown in Scheme IV, urea formation may involve reaction of a heteroaryl isocyanate (17) with an aryl amine (16). The heteroaryl isocyanate may be synthesized from a heteroaryl amine by treatment with phosgene or a phosgene equivalent, such as trichloromethyl chloroformate (diphosgene), bis(trichloromethyl) carbonate (triphosgene), or N,N′-carbonyldiimidazole (CDI). The isocyanate may also be derived from a heterocyclic carboxylic acid derivative, such as an ester, an acid halide or an anhydride by a Curtius-type rearrangement. Thus, reaction of acid derivative 21 with an azide source, followed by rearrangement affords the isocyanate. The corresponding carboxylic acid (22) may also be subjected to Curtius-type rearrangements using diphenylphosphoryl azide (DPPA) or a similar reagent. A urea may also be generated from the reaction of an aryl isocyanate (20) with a heterocyclic amine.

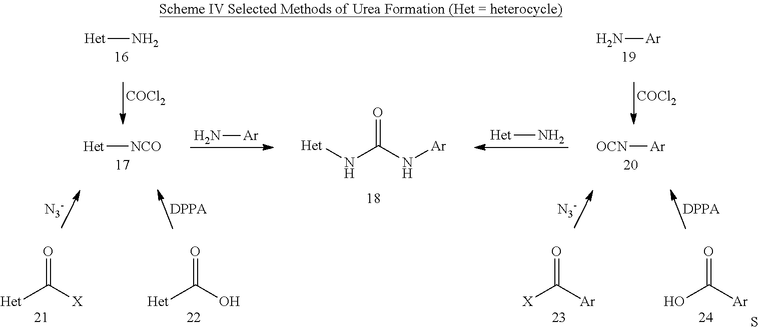

1-Amino-2-heterocyclic carboxylic esters (exemplified with thiophene 9, Scheme V) may be converted into an isatoic-like anhydride (25) through saponification, followed by treatment with phosgene or a phosgene equivalent, Reaction of anhydride 25 with an aryl amine can generate acid 26 which may spontaneously decarboxylate, or may be isolated. If isolated, decarboxylation of acid 26 may be induced upon heating.

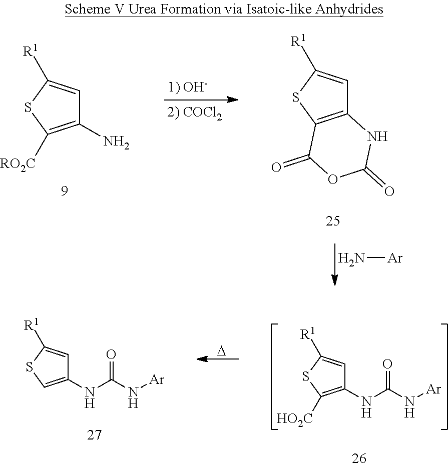

Finally, ureas may be further manipulated using methods familiar to those skilled in the art.

The invention also includes pharmaceutical compositions including a compound of Formula I or a pharmaceutically acceptable salt thereof, and a physiologically acceptable carrier.

The compounds may be administered orally, topically, parenterally, by inhalation or spray or sublingually, rectally or vaginally in dosage unit formulations. The term ‘administration by injection’ includes intravenous, intramuscular, subcutaneous and parenteral injections, as well as use of infusion techniques. Dermal administration may include topical application or transdermal administration. One or more compounds may be present in association with one or more non-toxic pharmaceutically acceptable carriers and if desired other active ingredients.

Compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally occurring phosphatide, for example, lecithin, or condensation products or an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds may also be administered in the form of suppositories for rectal or vaginal administration of the drug. These compositions can be prepared by mixing the drug with a suitable non-irritating excipient which is solid at ordinary temperatures but liquid at the rectal or vaginal temperature and will therefore melt in the rectum or vagina to release the drug. Such materials include cocoa butter and polyethylene glycols.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157 3Mar. 1994). For example, a solution or suspension of a compound of Formula I in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of Formula I may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C₈-C₁₈ fatty alcohols such as lauryl alcohol or cetyl alcohol, saturated or unsaturated C₈-C₁₈ fatty acids such as stearic acid, saturated or unsaturated fatty esters with up to 24 carbons such as methyl, ethyl, propyl, isopropyl, n-butyl, sec-butyl isobutyl tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C₈-C₁₈ fatty alcohols, saturated or unsaturated C₈-C₁₈ fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene coploymers, and natural and synthetic rubbers. Cellulose ethers, derivatized polyethylenes, and silicates may also be used as matrix components. Additional additives, such as viscous resins or oils may be added to increase the viscosity of the matrix.

For all regimens of use disclosed herein for compounds of Formula I, the daily oral dosage regimen will preferably be from 0.01 to 200 mg/Kg of total body weight. The daily dosage for administration by injection, including intravenous, intramuscular, subcutaneous and parenteral injections, and use of infusion techniques will preferably be from 0.01 to 200 mg/Kg of total body weight. The daily rectal dosage regime will preferably be from 0.01 to 200 mg/Kg of total body weight. The daily vaginal dosage regimen will preferably be from 0.01 to 200 mg/Kg of total body weight. The daily topical dosage regime will preferably be from 0.1 to 200 mg administered between one to four times daily. The transdermal concentration will preferably be that required to maintain a daily dose of from 0.01 to 200 mg/Kg. The daily inhalation dosage regime will preferably be from 0.01 to 10 mg/Kg of total body weight.

It will be appreciated by those skilled in the art that the particular method of administration will depend on a variety of factors, all of which are considered routinely when administering therapeutics.

It will also be understood, however, that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy.

It will be further appreciated by one skilled in the art that the optimal course of treatment, ie., the mode of treatment and the daily number of doses of a compound of Formula I or a pharmaceutically acceptable salt thereof given for a defined number of days, can be ascertained by those skilled in the art using conventional treatment tests.

It will be understood, however, that the specific dose level for any particular patient will depend upon a variety of factors, including the activity of the specific compound employed, the age, body weight, general health, sex, diet, time of administration, route of administration, and rate of excretion, drug combination and the severity of the condition undergoing therapy.

The entire disclosure of all applications, patents and publications cited above and below are hereby incorporated by reference, including provisional application Attorney Docket BAYER 8 V1, filed on Dec. 22, 1997, as Ser. No. 08/996,343, converted on Dec. 22, 1998.

The compounds are producible from known compounds (or from starting materials which, in turn, are producible from known compounds), e.g., through the general preparative methods shown below. The activity of a given compound to inhibit raf kinase can be routinely assayed, e.g., according to procedures disclosed below. The following examples are for illustrative purposes only and are not intended, nor should they be construed to limit the invention in any way.

### EXAMPLES

All reactions were performed in flame-dried or oven-dried glassware under a positive pressure of dry argon or dry nitrogen, and were stirred magnetically unless otherwise indicated. Sensitive liquids and solutions were transferred via syringe or cannula, and introduced into reaction vessels through rubber septa. Unless otherwise stated, the terra ‘concentration under reduced pressure’ refers to use of a Buchi rotary evaporator at approximately 15 mmHg.

All temperatures are reported uncorrected in degrees Celsius (° C.). Unless otherwise indicated, all parts and percentages are by weight.

Commercial grade reagents and solvents were used without further purification. Thin-layer chromatography (TLC) was performed on Whatman® pre-coated glass-backed silica gel 60A F-254 250 μm plates. Visualization of plates was effected by one or more of the following techniques: (a) ultraviolet illumination, (b) exposure to iodine vapor, (c) immersion of the plate in a 10% solution of phosphomolybdic acid in ethanol followed by heating, (d) immersion of the plate in a cerium sulfate solution followed by heating, and/or (e) immersion of the plate in an acidic ethanol solution of 2,4-dinitrophenylhydrazine followed by heating. Column chromatography (flash chromatography) was performed using 230-400 mesh EM Science® silica gel.

Melting points (mp) were determined using a Thomas-Hoover melting point apparatus or a Mettler FP66 automated melting point apparatus and are uncorrected. Fourier transform infrared spectra were obtained using a Mattson 4020 Galaxy Series spectrophotometer. Proton (¹H) nuclear magnetic resonance (NMR) spectra were measured with a General Electric GN-Omega 300 (300 MHz) spectrometer with either Me₄Si (δ 0.00) or residual protonated solvent (CHCl₃ δ 7.26; MeOH δ 3.30; DMSO δ 2.49) as standard. Carbon (¹³C) NMR spectra were measured with a General Electric GN-Omega 300 (75 MHz) spectrometer with solvent (CDCl₃ δ 77.0; MeOD-d₃; δ 49.0; DMSO-d₆ δ 39.5) as standard. Low resolution mass spectra (MS) and high resolution mass spectra (HRMS) were either obtained as electron impact (EI) mass spectra or as fast atom bombardment (FAB) mass spectra. Electron impact mass spectra (EI-MS) were obtained with a Hewlett Packard 5989A mass spectrometer equipped with a Vacumetrics Desorption Chemical Ionization Probe for sample introduction. The ion source was maintained at 250° C. Electron impact ionization was performed with electron energy of 70 eV and a trap current of 300 μA. Liquid-cesium secondary ion mass spectra (FAB-MS), an updated version of fast atom bombardment were obtained using a Kratos Concept 1-H spectrometer. Chemical ionization mass spectra (CI-MS) were obtained using a Hewlett Packard MS-Engine (5989A) with methane as the reagent gas (1×10⁻⁴ torr to 2.5×10⁻⁴ torr). The direct insertion desorption chemical ionization (DCI) probe (Vaccumetrics, Inc.) was ramped from 0-1.5 amps in 10 sec and held at 10 amps until all traces of the sample disappeared (˜1-2 min). Spectra were scanned from 50-800 amu at 2 sec per scan. HPLC—electrospray mass spectra (HPLC ES-MS) were obtained using a Hewlett-Packard 1100 HPLC equipped with a quaternary pump, a variable wavelength detector, a C-18 column, and a Finnigan LCQ ion trap mass spectrometer with electrospray ionization. Spectra were scanned from 120-800 amu using a variable ion time according to the number of ions in the source. Gas chromatography—ion selective mass spectra (GC-MS) were obtained with a Hewlett Packard 5890 gas chromatograph equipped with an HP-1 methyl silicone column (0.33 mM coating; 25 m×0.2 mm) and a Hewlett Packard 5971 Mass Selective Detector (ionization energy 70 eV).

Elemental analyses were conducted by Robertson Microlit Labs, Madison N.J. All ureas displayed NMR spectra, LRMS and either elemental analysis or HRMS consistant with assigned structures.

### List of Abbreviations and Acronyms:

AcOH acetic acid
anh anhydrous
BOC tert-butoxycarbonyl
cone concentrated
dec decomposition
DMPU 1,3-dimethyl-3,4,5,6-tetrahydro-2(1H)-pyrimidinone

### DMF N,N-dimethylformamide

DMSO dimethylsulfoxide
DPPA diphenylphosphoryl azide
EtOAc ethyl acetate
EtOH ethanol (100%)
Et₂O diethyl ether
Et₃N triethylamine
m-CPBA 3-chloroperoxybenzoic acid
MeOH methanol
pet. ether petroleum ether (boiling range 30-60° C.)
THF tetrahydrofuran
TFA trifluoroacetic acid
Tf trifluoromethanesulfonyl

### A. General Methods for Synthesis of Hetrocyclic Amines

A2. General Synthesis of 5-Amino-3-alkylisoxazoles

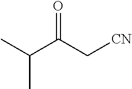

Step 1. 3-Oxo-4-methylpentanenitrile: A slurry of sodium hydride (60% in mineral oil; 10.3 g, 258 mmol) in benzene (52 mL) was warmed to 80° C. for 15 min., then a solution of acetonitrile (115 mL, 258 mmol) in benzene (52 mL) was added dropwise via addition funnel followed by a solution of ethyl isobutyrate (15 g, 129 mmol) in benzene (52 mL). The reaction mixture was heated overnight, then cooled with an ice water bath and quenched by addition of 2-propanol (50 mL) followed by water (50 mL) via addition funnel. The organic layer was separated and set aside. EtOAc (100 mL) was added to the aqueous layer and the resulting mixture was acidified to approximately pH 1 (conc. HCl) with stirring. The resulting aqueous layer was extracted with EtOAc (2×100 mL). The organic layers were combined with the original organic layer, dried (MgSO₄), and concentrated in vacuo to give the a-cyanoketone as a yellow oil which was used in the next step without further purification.

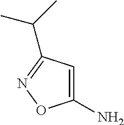

Step 2. 5-Amino-3-isopropylisoxazole: Hydroxylamine hydrochloride (10.3 g, 148 mmol) was slowly added to an ice cold solution of NaOH (25.9 g, 645 mmol) in water (73 mL) and the resulting solution was poured into a solution of crude 3-oxo-4-methylpentanenitrile while stirring. The resulting yellow solution was heated at 50° C. for 2.5 hours to produce a less dense yellow oil. The warm reaction mixture was immediately extracted with CHCl₃ (3×100 mL) without cooling. The combined organic layers were dried (MgSO₄), and concentrated in vacuo. The resulting oily yellow solid was filtered through a pad of silica (10% acetone/90% CH₂Cl₂) to afford the desired isoxazole as a yellow solid (11.3 g, 70%): mp 63-65° C.; TLC R_{f} (5% acetone/95% CH₂Cl₂) 0.19; ¹H-NMR (DMSO-d₆) d 1.12 (d, J=7.0 Hz, 6H), 2.72 (sept, J=7.0 Hz, 1H), 4.80 (s, 2H), 6.44 (s, 1H); FAB-MS m/z (rel abundance) 127 ((M+H)⁺; 67%).

A3. General Method for the Preparation of 5-Amino-1-alkyl-3-alkylpyrazoles

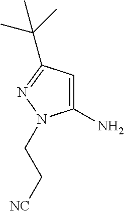

5-Amino-3-tert-butyl-1-(2-cyanoethyl)pyrazole: A solution of 4,4-dimethyl-3-oxopentanenitrile (5.6 g, 44.3 mmol) and 2-cyanoethyl hydrazine (4.61 g, 48.9 mmol) in EtOH (100 mL) was heated at the reflux temperature overnight after which TLC analysis showed incomplete reaction. The mixture was concentrated under reduced pressure and the residue was filtered through a pad of silica (gradient from 40% BtOAc/60% hexane to 70% EtOAc/30% hexane) and the resulting material was triturated (Et₂O/hexane) to afford the desired product (2.5 g, 30%): TLC (30% EtOAc/70% hexane) R_{f} 0.31; (DMSO-d₆) δ 1.13 (s, 9H), 2.82 (t, J=6.9 Hz, 2H), 4.04 (t, J=6.9 Hz, 2H), 5.12 (br s, 2H), 5.13 (s, 1H).

A 4. Synthesis of 3-Amino-5-alkylthiophenes
A4a. Synthesis of 3-Amino-5-alkylthiophenes by Thermal Decarboxylation of Thiophenecarboxylic Acids

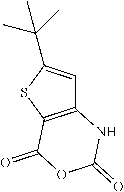

Step 1. 7-tert-Butyl-2H-thieno[3,2-d]oxazine-2,4(1H)-dione: A mixture of methyl 3-amino-5-tert-butylthiophenecarboxylate (7.5 g, 35.2 mmol) and KOH (5.92 g) in MeOH (24 mL) and water (24 mL) was stirred at 90° C. for 6 h. The reaction mixture was concentrated under reduced pressure and the residue was dissolved in water (600 mL). Phosgene (20% in toluene, 70 mL) was added dropwise over a 2 h period. The resulting mixture was stirred at room temperature overnight and the resulting precipitate was triturated (acetone) to afford the desired anhydride (5.78 g, 73%): ¹H-NMR (CDCl₃) δ 1.38 (s, 9H), 2.48 (s, 1H), 6.75 (s, 1H); FAB-MS m/z (rel abundance) 226 ((M+H)⁺, 100%).

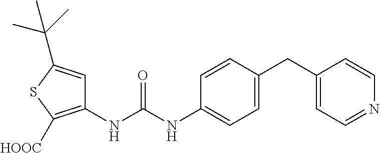

Step 2. N-(5-tert-Butyl-2-carboxy-3-thienyl)-N′-(4-(4-pyridinylmethyl)phenyl)-urea: A solution of 7-tert-butyl-2H-thieno[3,2-d]oxazine-2,4(1H)-dione (0.176 g, 0.78 mmol) and 4-(4-pyridinylmethyl)aniline (0.144 g, 0.78 mmol) in THF (5 mL) was heated at the reflux temp. for 25 h. After cooling to room temp., the resulting solid was triturated with Et₂O to afford the desired urea (0.25 g, 78%): nip 187-189° C.; TLC (50% EtOAc/50% pet. ether) R_{f} 0.04; ¹H-NMR (DMSO-d₆) δ 1.34 (s, 9H), 3.90 (s, 2H), 7.15 (d, J=7 Hz, 2H), 7.20 (d, J=3 Hz, 2H), 7.40 (d, J=7 Hz, 2H), 7.80 (s 1H), 8.45 (d, J=3 Hz, 2H) 9.55 (s, 1H), 9.85 (s, 1H), 12.50 (br s, 1H); FAB-MS m/z (rel abundance) 410 ((M+H)⁺; 20%).

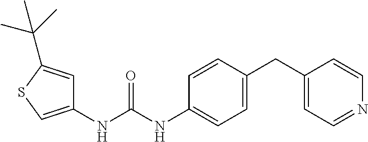

Step 3. N-(5-tert-Butyl-3-thienyl)-N′-(4-(4-pyridinylmethyl)phenyl)urea: A vial containing N-(5-tert-butyl-2-carboxy-3-thienyl)-N′-(4-(4-pyridinylmethyl)phenyl)-urea (0.068 g, 0.15 mmol) was heated to 199° C. in an oil bath. After gas evolution ceased, the material was cooled and purified by preparative HPLC (C-18 column; gradient from 20% CH₅CN/79.9% H₂O/0.1% TFA to 99.9% H₂O/0.1% TFA) to give the desired product (0.024 g, 43%): TLC (50% EtOAc/50% pet. ether) R_{f} 0.18; ¹H-NMR (DMSO-d₆) δ 1.33 (s, 9H), 4.12 (s, 2H), 6.77 (s, 1H), 6.95 (s, 1H), 7.17 (d, J=9 Hz, 2H), 7.48 (d, J=9 Hz, 2H), 7.69 (d, J=7 Hz, 1H), 8.58 (s, 1H), 8.68 (d, J=7 Hz, 2H), 8.75 (s, 1H); EI-MS m/z 365 (M⁺).

A4b. Synthesis 3-Amino-5-alkylthiophenes from 3-Amino-5-alkyl-2-thiophene-carboxylate esters

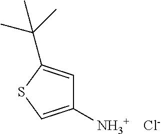

5-tert-Butyl-3-thiopheneammonium Chloride: To a solution of methy 3-amino-5-tert-butyl-2-thiophene-carboxylate (5.07 g, 23.8 mmol, 1.0 equiv) in EtOH (150 mL) was added NaOH (2.0 g, 50 mmol, 2.1 equiv). The resulting solution was heated at the reflux temp. for 2.25 h. A conc. HCl solution (approximately 10 mL) was added dropwise with stirring and the evolution of gas was observed. Stirring was continued for 1 h, then the solution was concentrated under reduced pressure. The white residue was suspended in EtOAc (150 mL) and a saturated NaHCO₃ solution (150 mL) was added to dissolve. The organic layer was washed with water (150 mL) and a saturated NaCl solution (150 mL), dried (Na₂SO₄), and concentrated under reduced pressure to give the desired ammonium salt as a yellow oil (3.69 g, 100%). This material was used directly in urea formation without further purification.

A4c. Synthesis 3-Amino-5-alkylthiophenes from N-BOC 3-Amino-5-alkyl-2-thiophenecarboxylate esters

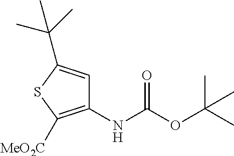

Step 1. Methyl 3-(tert-Butoxycarbonylamino)-5-tert-butyl-2-thiophenecarboxylate: To a solution of methyl 3-amino-5-tert-butyl-2-thiophenecarboxylate (150 g, 0.70 mol) in pyridine (2.8 L) at 5° C. was added di-tert-butyl dicarbonate (171.08 g, 0.78 mol, 1.1 equiv) and N,N-dimethylaminopyridine (86 g, 0.70 mol, 1.00 equiv) and the resulting mixture was stirred at room temp for 7 d. The resulting dark solution was concentrated under reduced pressure (approximately 0.4 mmHg) at approximately 20° C. The resulting red solids were dissolved in CH₂Cl₂ (3 L) and sequentially washed with a 1 M H₃PO₄ solution (2×750 mL), a saturated NaHCO₃ solution (800 mL) and a saturated NaCl solution (2×800 mL), dried (Na₂SO₄) and concentrated under reduced pressure. The resulting orange solids were dissolved in abs. EtOH (2 L) by warming to 49° C., then treated with water (500 mL) to afford the desired product as an off-white solid (163 g, 74%): ¹H-NMR (CDCl₃) δ 1.38 (s, 9H), 1.51 (s, 9H), 3.84 (s, 3H), 7.68 (s, 1H), 9.35 (br s, 1H); FAB-MS m/z (rel abundance) 314 ((M+H)⁺, 45%).

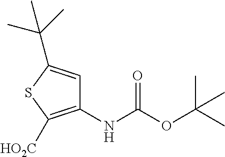

Step 2. 3-(tert-Butoxycarbonylamino)-5-tert-butyl-2-thiophenecarboxylic Acid: To a solution of methyl 3-(tert-butoxycarbonylamino)-5-tert-butyl-2-thiophenecarboxylate (90.0 g, 0.287 mol) in THF (630 mL) and MeOH (630 mL) was added a solution of NaOH (42.5 g, 1.06 mL) in water (630 mL). The resulting mixture was heated at 60° C. for 2 h, concentrated to approximately 700 mL under reduced pressure, and cooled to 0° C. The pH was adjusted to approximately 7 with a 1.0 N HCl solution (approximately 1 L) while maintaining the internal temperature at approximately 0° C. The resulting mixture was treated with EtOAc (4 L). The pH was adjusted to approximately 2 with a 1.0 N HCl solution (500 mL). The organic phase was washed with a saturated NaCl solution (4×1.5 L), dried (Na₂SO₄), and concentrated to approximately 200 mL under reduced pressure. The residue was treated with hexane (1 L) to form a light pink (41.6 g). Resubmission of the mother liquor to the concentration-precipitation protocol afforded additional product (38.4 g, 93% total yield): ¹H-NMR (CDCl₃) δ 1.94 (s, 9H), 1.54 (s, 9H), 7.73 (s, 1H), 9.19 (br s, 1H); FAB-MS m/z (rel abundance) 300 ((M+H)⁺, 50%).

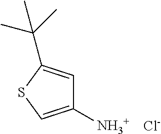

Step 3. 5-tert-Butyl-3-thiopheneammonium Chloride: A solution of 3-(tert-butoxycarbonylamino)-5-tert-butyl-2-thiophenecarboxylic acid (3.0 g, 0.010 mop in dioxane (20 mL) was treated with an HCl solution (4.0 M in dioxane, 12.5 mL, 0.050 mol, 5.0 equiv), and the resulting mixture was heated at 80° C. for 2 h. The resulting cloudy solution was allowed to cool to room temp forming some precipitate. The slurry was diluted with EtOAc (50 mL) and cooled to −20° C. The resulting solids were collected and dried overnight under reduced pressure to give the desired salt as an off-white solid (1.72 g, 90%): ¹H-NMR (DMSO-d₆) δ 1.31 (s, 9H), 6.84 (d, J=1.48 Hz, 1H), 7.31 (d, J=1.47 Hz, 1H), 10.27 (br s, 3H).

### A5. General Method for the Synthesis of BOC-Protected Pyrazoles

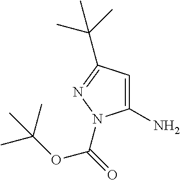

5-Amino-3-tert-butyl-N¹-(tert-butoxycarbonyl)pyrazole: To a solution of 5-amino-3-tert-butylpyrazole (3.93 g, 28.2 mmol) in CH₂Cl₂ (140 mL) was added di-tert-butyl dicarbonate (6.22 g, 28.5 mmol) in one portion. The resulting solution was stirred at room temp. for 13 h, then diluted with EtOAc (500 mL). The organic layer was washed with water (2×300 mL), dried (MgSO₄) and concentrated under reduced pressure. The solid residue was triturated (100 mL hexane) to give the desired carbamate (6.26 g, 92%): mp 63-64° C.; TLC R_{f} (5% acetone/95% CH₂Cl₂); ¹H-NMR (DMSO-d₆) δ 1.15 (s, 9H), 1.54 (s, 9H), 5.22 (s, 1H), 6.11 (s, 2H); FAB-MS m/z ((M+H)⁺).

### A6. General Method for the Synthesis of 2-Aminothiadiazoles

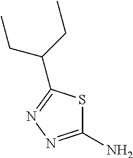

2-Amino-5-(1-(1-ethyl)propyl)thiadiazine: To concentrated sulfuric acid (9.1 mL) was slowly added 2-ethylbutyric acid (10.0 g, 86 mmol, 1.2 equiv). To this mixture was slowly added thiosemicarbazide (6.56 g, 72 mmol, 1 equiv). The reaction mixture was heated at 85° C. for 7 h, then cooled to room temperature, and treated with a concentrated NH₄OH solution until basic. The resulting solids were filtered to afford 2-amino-5-(1-(1-ethyl)propyl)thiadiazine product was isolated via vacuum filtration as a beige solid (6.3 g, 51%): mp 155-158° C.; TLC (5% MeOH/95% CHCl₃) R_{f} 0.14; ¹H-NMR (DMSO-d₆) δ 0.80 (t, J=7.35 Hz, 6H), 1.42-4.60 (m, 2H), 1.59-1.71 (m, 2H), 2.65-2.74 (m, 1H), 7.00 (br s, 2H); HPLC ES-MS m/z 172 ((M+H)⁺).

### A7. General Method for the Synthesis of 2-Aminooxadiazoles

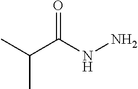

Step 1. Isobutyric Hydrazide: A solution of methyl isobutyrate (10.0 g) and hydrazine (2.76 g) in MeOH (500 mL) was heated at the reflux temperature over night then stirred at 60° C. for 2 weeks. The resulting mixture was cooled to room temperature and concentrated under reduced pressure to afford isobutyric hydrazide as a yellow oil (1.0 g, 10%), which was used in the next step without further purification.

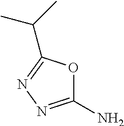

Step 2. 2-Amino-5-isopropyl oxadiazole: To a mixture of isobutyric hydrazide (0.093 g), KHCO₃ (0.102 g), and water (1 mL) in dioxane (1 mL) at room temperature was added cyanogen bromide (0.10 g). The resulting mixture was heated at the reflux temperature for 5 h, and stirred at room temperature for 2 d, then treated with CH₂Cl₂ (5 mL). The organic layer was washed with water (2×10 mL), dried (MgSO₄) and concentrated under reduced pressure to afford 2-amino-5-isopropyl oxadiazole as a white solid: HPLC ES-MS m/z 128 ((M+H)⁺).

### A8. General Method for the Synthesis of 2-Aminooxazoles

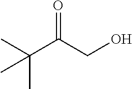

Step 1. 3,3-Dimethyl-1-hydroxy-2-butanone: A neat sample of 1-bromo-3,3-dimethyl-2-butanone (33.3 g) at 0° C. was treated with a 1N NaOH solution, then was stirred for 1 h. The resulting mixture was extracted with EtOAc (5×100 mL). The combined organics were dried (Na₂SO₄) and concentrated under reduced pressure to give 3,3-dimethyl-1-hydroxy-2-butanone (19 g, 100%), which was used in the next step without further purification.

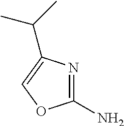

Step 2. 2-Amino-4-isopropyl-1,3-oxazole: To a solution of 3,3-dimethyl-1-hydroxy-2-butanone (4.0 g) and cyanimide (50% w/w, 2.86 g) in THF (10 mL) was added a 1N NaOAc solution (8 mL), followed by tetra-n-butylammonium hydroxide (0.4 M, 3.6 mL), then a 1N NaOH solution (1.45 mL). The resulting mixture was stirred at room temperature for 2 d. The resulting organic layer was separated, washed with water (3×25 mL), and the aqueous layer was extraced with Et₂O (3×25 L). The combined organic layers were treated with a 1N NaOH solution tuntil basic, then extracted with CH₂Cl₂ (3×25 mL). The combined organic layers were dried (Na₂SO₄) and concentrated under reduced pressure to afford 2-Amino-4-isopropyl-1,3-oxazole (1.94 g, 41%): HPLC ES-MS m/z 141 ((M+H)⁺).

A9. Method for the Synthesis of Substituted-5-aminotetrazoles

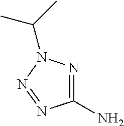

: To a solution of 5-aminotetrazole (5 g), NaOH (2.04 g) and water (25 mL) in EtOH (115 mL) at the reflux temperature was added 2-bromopropane (5.9 g). The resulting mixture was heated at the reflux temperature for 6 d, then cooled to room temperature, and concentrated under reduced pressure. The resulting aqueous mixture was washed with CH₂Cl₂ (3×25 mL), then concentrated under reduced pressure with the aid of a lyophlizer to afford a mixture of 1- and 2-isopropyl-5-aminotetrazole (50%), which was used without further purification: HPLC ES-MS m/z 128 ((M+H)⁺).

### B. General Methods for Synthesis of Substituted Anilines

### B1. General Method for Substituted Aniline Formation via Hydrogenation of a Nitroarene

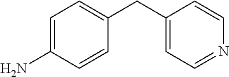

4-(4-Pyridinylmethyl)aniline: To a solution of 4-(4-nitrobenzyl)pyridine (7.0 g, 32.68 mmol) in EtOH (200 mL) was added 10% Pd/C (0.7 g) and the resulting slurry was shaken under a H₂ atmosphere (50 psi) using a Parr shaker. After 1 h, TLC and ¹H-NMR of an aliquot indicated complete reaction. The mixture was filtered through a short pad of Celite®. The filtrate was concentrated in vacuo to afford a white solid (5.4 g, 90%): ¹H-NMR (DMSO-d₆) δ 3.74 (s, 2H), 4.91 (hr s, 2H), 6.48 (d, J=8.46 Hz, 2H), 6.86 (d, J=8.09 Hz, 2H), 7.16 (d, J=5.88 Hz, 2H), 8.40 (d, J=5.88 Hz, 2H); EI-MS m/z 184 (M⁺). This material was used in urea formation reactions without further purification.

### B2. General Method for Substituted Aniline Formation via Dissolving Metal Reduction of a Nitroarene

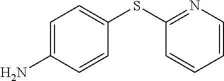

4-(2-Pyridinylthio)aniline: To a solution of 4-(2-pyridinylthio)-1-nitrobenzene (Menai ST 3355A; 0.220 g, 0.95 mmol) and H₂O (0.5 mL) in AcOH (5 mL) was added iron powder (0.317 g, 5.68 mmol) and the resulting slurry stirred for 16 h at room temp. The reaction mixture was diluted with EtOAc (75 mL) and H₂O (50 mL), basified to pH 10 by adding solid K₂CO₃ in portions (Caution: foaming). The organic layer was washed with a saturated NaCl solution, dried (MgSO₄), concentrated in vacuo. The residual solid was purified by MPLC (30% EtOAc/70% hexane) to give the desired product as a thick oil (0.135 g, 70%): TLC (30% EtOAc/70% hexanes) R_{f} 0.20.

B3a. General Method for Substituted Aniline Formation via Nitroarene Formation Through Nucleophilic Aromatic Substitution, Followed by Reduction

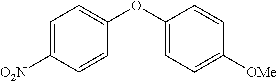

Step 1. 1-Methoxy-4-(4-nitrophenoxy)benzene: To a suspension of NaH (95%, 1.50 g, 59 mmol) in DMF (100 mL) at room temp. was added dropwise a solution of 4-methoxyphenol (739 g, 59 mmol) in DMF (50 mL). The reaction was stirred 1 h, then a solution of 1-fluoro-4-nitrobenzene (7.0 g, 49 mmol) in DMF (50 mL) was added dropwise to form a dark green solution. The reaction was heated at 95° C. overnight, then cooled to room temp., quenched with H₂O, and concentrated in vacuo. The residue was partitioned between EtOAc (200 mL) and H₂O (200 mL). The organic layer was sequentially washed with H₂O (2×200 mL), a saturated NaHCO₃ solution (200 mL), and a saturated NaCl solution (200 mL), dried (Na₂SO₄), and concentrated in vacuo. The residue was triturated (Et₂O/hexane) to afford 1-methoxy-4-(4-nitrophenoxy)benzene (12.2 g, 100%): ¹H-NMR (CDCl₃) δ 3.83 (s, 3H), 6.93-7.04 (m, 6H), 8.18 (d, J=9.2 Hz, 2H); EI-MS m/z 245 (M⁺).

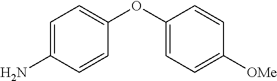

Step 2. 4-(4-Methoxyphenoxy)aniline: To a solution of 1-methoxy-4-(4-nitrophenoxy)benzene (12.0 g, 49 mmol) in EtOAc (250 mL) was added 5% Pt/C (1.5 g) and the resulting slurry was shaken under a H₂ atmosphere (50 psi) for 18 h.

The reaction mixture was filtered through a pad of Celite® with the aid of EtOAc and concentrated in vacuo to give an oil which slowly solidified (10.6 g, 100%): ¹H-NMR (CDCl₃) δ 3.54 (br s, 2H), 3.78 (s, 3H), 6.65 (d, J=8.8 Hz, 2H), 6.79-6.92 (m, 6H); EI-MS m/z 215 (M⁺).

B3b. General Method for Substituted Aniline Formation via Nitroarene Formation Through Nucleophilic Aromatic Substitution, Followed by Reduction

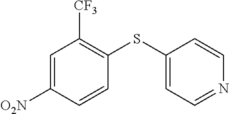

Step 1. 3-(Trifluoromethyl)-4-(4-pyridinylthio)nitrobenzene: A solution of 4-mercaptopyridine (2.8 g, 24 mmoles), 2-fluoro-5-nitrobenzotrifluoride (5 g, 23.5 mmoles), and potassium carbonate (6.1 g, 44.3 mmoles) in anhydrous DMF (80 mL) was stirred at room temperature and under argon overnight. TLC showed complete reaction. The mixture was diluted with Et₂O (100 mL) and water (100 mL) and the aqueous layer was back-extracted with Et₂O (2×100 mL). The organic layers were washed with a saturated NaCl solution (100 mL), dried (MgSO₄), and concentrated under reduced pressure. The solid residue was triturated with Et₂O to afford the desired product as a tan solid (3.8 g, 54%): TLC (30% EtOAc/70% hexane) R_{f} 0.06; ¹H-NMR (DMSO-d₆) δ 7.33 (dd, J=1.2, 4.2 Hz, 2H), 7.78 (d, Hz, 1H), 8.46 (dd, J=2.4, 8.7 Hz, 1H), 8.54-8.56 (m, 3H).

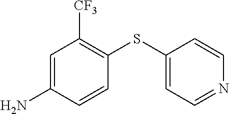

Step 2. 3-(Trifluoromethyl)-4-(4-pyridinylthio)aniline: A slurry of 3-trifluoromethyl-4-(4-pyridinylthio)nitrobenzene (3.8 g, 12.7 mmol), iron powder (4.0 g, 71.6 mmol), acetic acid (100 mL), and water (1 mL) were stirred at room temp. for 4 h. The mixture was diluted with Et₂O (100 mL) and water (100 mL). The aqueous phase was adjusted to pH 4 with a 4 N NaOH solution. The combined organic layers were washed with a saturated NaCl solution (100 mL), dried (MgSO₄), and concentrated under reduced pressure. The residue was filtered through a pad of silica (gradient from 50% EtOAc/50% hexane to 60% EtOAc/40% hexane) to afford the desired product (3.3 g): TLC (50% EtOAc/50% hexane) R_{f} 0.10; ¹H-NMR (DMSO-d₆) δ 6.21 (s, 2H), 6.84-6.87 (m, 3H), 7.10 (d, Hz, 1H), 7.39 (d, J=8.4 Hz, 1H), 8.29 (d, J=6.3 Hz, 2H).

B3c. General Method for Substituted Aniline Formation via Nitroarene Formation Through Nucleophilic Aromatic Substitution, Followed by Reduction

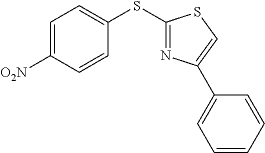

Step 1. 4-(2-(4-Phenyl)thiazolyl)thio-1-nitrobenzene: A solution of 2-mercapto-4-phenylthiazole (4.0 g, 207 mmoles) in DMF (40 mL) was treated with 1-fluoro-4-nitrobenzene (2.3 mL, 21.7 mmoles) followed by K₂CO₃ (3.18 g, 23 mmol), and the mixture was heated at approximately 65° C. overnight. The reaction mixture was then diluted with EtOAc (100 mL), sequentially washed with water (100 mL) and a saturated NaCl solution (100 mL), dried (MgSO₄) and concentrated under reduced pressure. The solid residue was triturated with a Et₂O/hexane solution to afford the desired product (6.1 g): TLC (25% EtOAc/75% hexane) R_{f} 0.49; ¹H-NMR (CDCl₃) δ 7.35-7.47 (m, 3H), 7.58-7.63 (m, 3H), 7.90 (d, Hz, 2H), 8.19 (d, J=9.0 Hz, 2H).

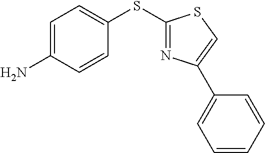

Step 2. 4-(2-(4-Phenyl)thiazolyl)thioaniline: 4-(2-(4-Phenyl)thiazolyl)thio-1-nitro-benzene was reduced in a manner analagous to that used in the preparation of 3-(trifluoromethyl)-4-(4-pyridinylthio)aniline: TLC (25% EtOAc/75% hexane) R_{f} 0.18; ¹H-NMR (CDCl₃) δ 3.89 (br s, 2H), 6.72-6.77 (m, 2H), 7.26-7.53 (m, 6H), 7.85-7.89 (m, 2H).

B3d. General Method for Substituted Aniline Formation via Nitroarene Formation Through Nucleophilic Aromatic Substitution, Followed by Reduction

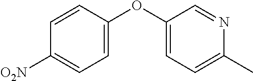

Step 1. 4-(6-Methyl-3-pyridinyloxy)-1-nitrobenzene: To a solution of 5-hydroxy-2-methylpyridine (5.0 g, 45.8 mmol) and 1-fluoro-4-nitrobenzene (6.5 g, 45.8 mmol) in arch DMF (50 mL) was added K₂CO₃ (13.0 g, 91.6 mmol) in one portion. The mixture was heated at the reflux temp. with stirring for 18 h and then allowed to cool to room temp. The resulting mixture was poured into water (200 mL) and extracted with EtOAc (3×150 mL). The combined organics were sequentially washed with water (3×100 mL) and a saturated NaCl solution (2×100 mL), dried (Na₂SO₄), and concentrated in vacuo to afford the desired product (8.7 g, 83%). The this material was carried to the next step without further purification.

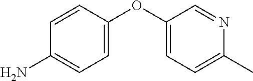

Step 2. 4-(6-Methyl-3-pyridinyloxy)aniline: A solution of 4-(6-methyl-3-pyridinyloxy)-1-nitrobenzene (4.0 g, 17.3 mmol) in EtOAc (150 mL) was added to 10% Pd/C (0.500 g, 0.47 mmol) and the resulting mixture was placed under a H₂ atmosphere (balloon) and was allowed to stir for 18 h at room temp. The mixture was then filtered through a pad of Celite® and concentrated in vacuo to afford the desired product as a tan solid (3.2 g, 92%): EI-MS m/z 200 (M⁺).

B3e. General Method for Substituted Aniline Formation via Nitroarene Formation Through Nucleophilic Aromatic Substitution, Followed by Reduction

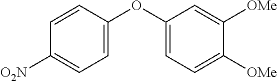

Step 1. 4-(3,4-Dimethoxyphenoxy)-1-nitrobenzene: To a solution of 3,4-dimethoxyphenol (1.0 g, 6.4 mmol) and 1-fluoro-4-nitrobenzene (700 μL, 6.4 mmol) in anh DMF (20 mL) was added K₂CO₃ (1.8 g, 12.9 mmol) in one portion. The mixture was heated at the reflux temp with stirring for 18 h and then allowed to cool to room temp. The mixture was then poured into water (100 mL) and extracted with EtOAc (3×100 mL). The combined organics were sequentially washed with water (3×50 mL) and a saturated NaCl solution (2×50 mL), dried (Na₂SO₄), and concentrated in vacuo to afford the desired product (0.8 g, 54%). The crude product was carried to the next step without further purification.

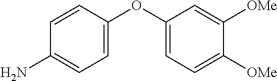

Step 2. 4-(3,4-Dimethoxyphenoxy)aniline: A solution of 4-(3,4-dimethoxy-phenoxy)-1-nitrobenzene (0.8 g, 3.2 mmol) in EtOAc (50 mL) was added to 10% Pd/C (0.100 g) and the resulting mixture was placed under a H₂ atmosphere (balloon) and was allowed to stir for 18 h at room temp. The mixture was then filtered through a pad of Celite® and concentrated in vacuo to afford the desired product as a white solid (0.6 g, 75%): EI-MS m/z 245 (M⁺).

B3f. General Method for Substituted Aniline Formation via Nitroarene Formation Through Nucleophilic Aromatic Substitution, Followed by Reduction

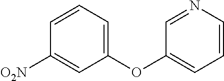

Step 1. 3-(3-Pyridinyloxy)-1-nitrobenzene: To a solution of 3-hydroxypyridine (2.8 g, 29.0 mmol), 1-bromo-3-nitrobenzene (5.9 g, 29.0 mmol) and copper(I) bromide (5.0 g, 34.8 mmol) in anh DMF (50 mL) was added K₂CO₃ (8.0 g, 58.1 mmol) in one portion. The resulting mixture was heated at the reflux temp. with stirring for 18 h and then allowed to cool to room temp. The mixture was then poured into water (200 mL) and extracted with EtOAc (3×150 mL). The combined organics were sequentially washed with water (3×100 mL) and a saturated NaCl solution (2×100 mL), dried (Na₂SO₄), and concentrated in vacuo. The resulting oil was purified by flash chromatography (30% EtOAc/70% hexane) to afford the desired product (2.0 g, 32%). This material was used in the next step without further purification.

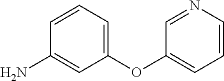

Step 2. 3-(3-Pyridinyloxy)aniline: A solution of 3-(3-pyridinyloxy)-1-nitrobenzene (2.0 g, 9.2 mmol) in EtOAc (100 mL) was added to 10% Pd/C (0.200 g) and the resulting mixture was placed under a H₂ atmosphere (balloon) and was allowed to stir for 18 h at room temp. The mixture was then filtered through a pad of Celite® and concentrated in vacuo to afford the desired product as a red oil (1.6 g, 94%): EI-MS m/z 186 (M⁺).

B3g. General Method for Substituted Aniline Formation via Nitroarene Formation Through Nucleophilic Aromatic Substitution, Followed by Reduction

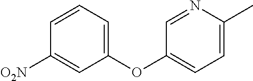

Step 1. 3-(5-Methyl-3-pyridinyloxy)-1-nitrobenzene: To a solution of 3-hydroxy-5-methylpyridine (5.0 g, 45.8 mmol), 1-bromo-3-nitrobenzene (12.0 g, 59.6 mmol) and copper(I) iodide (10.0 g, 73.3 mmol) in anh DMF (50 mL) was added K₂CO₃ (13.0 g, 91.6 mmol) in one portion. The mixture was heated at the reflux temp. with stirring for 18 h and then allowed to cool to room temp. The mixture was then poured into water (200 mL) and extracted with EtOAc (3×150 mL). The combined organics were sequentially washed with water (3×100 mL) and a saturated NaCl solution (2×100 mL), dried (Na₂SO₄), and concentrated in vacuo. The resulting oil was purified by flash chromatography (30% EtOAc/70% hexane) to afford the desired product (1.2 g, 13%).

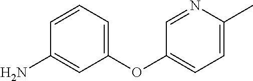

Step 2. 3-(5-Methyl-3-pyridinyloxy)-1-nitrobenzene: A solution of 3-(5-methyl-3-pyridinyloxy)-1-nitrobenzene (1.2 g, 5.2 mmol) in EtOAc (50 mL) was added to 10% Pd/C (0.100 g) and the resulting mixture was placed under a H₂ atmosphere (balloon) and was allowed to stir for 18 h at room temp. The mixture was then filtered through a pad of Celite® and concentrated in vacuo to afford the desired product as a red oil (0.9 g, 86%): CI-MS m/z 201 ((M+H)⁺).

2B3h. General Method for Substituted Aniline Formation via Nitroarene Formation Through Nucleophilic Aromatic Substitution, Followed by Reduction

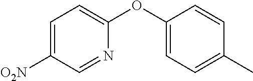

Step 1. 5-Nitro-2-(4-methylphenoxy)pyridine: To a solution of 2-chloro-5-nitropyridine (6.34 g, 40 mmol) in DMF (200 mL) were added of 4-methylphenol (5.4 g, 50 mmol, 1.25 equiv) and K₂CO₃ (8.28 g, 60 mmol, 1.5 equiv). The mixture was stirred overnight at room temp. The resulting mixture was treated with water (600 mL) to generate a precipitate. This mixture was stirred for 1 h, and the solids were separated and sequentially washed with a 1 N NaOH solution (25 mL), water (25 mL) and pet ether (25 mL) to give the desired product (7.05 g, 76%): mp 80-82° C.; TLC (30% EtOAc/70% pet ether) R_{f} 0.79; ¹H-NMR (DMSO-d₆) δ 2.31 (s, 3H), 7.08 (d, J=8.46 Hz, 2H), 7.19 (d, J=9.20 Hz, 1H), 7.24 (d, J=8.09 Hz, 2H), 8.58 (dd, J=2.94, 8.82 Hz, 1H), 8.99 (d, J=2.95 Hz, 1H); FAB-MS m/z (rel abundance) 231 ((M+H)⁺), 100%).

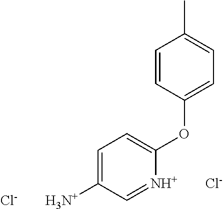

Step 2. 5-Amino-2-(4-methylphenoxy)pyridine Dihydrochloride: A solution 5-nitro-2-(4-methylphenoxy)pyridine (6.94 g, 30 mmol, 1 eq) and EtOH (10 mL) in EtOAc (190 mL) was purged with argon then treated with 10% Pd/C (0.60 g). The reaction mixture was then placed under a H₂ atmosphere and was vigorously stirred for 2.5 h. The reaction mixture was filtered through a pad of Celite®. A solution of HCl in Et₂O was added to the filtrate was added dropwise. The resulting precipitate was separated and washed with EtOAc to give the desired product (7.56 g, 92%): mp 208-210° C. (dec); TLC (50% EtOAc/50% pet ether) R_{f} 0.42; ¹H-NMR (DMSO-d₆) δ 2.25 (s, 3H), 6.98 (d, J=8.45 Hz, 2H), 7.04 (d, J=8.82 Hz, 1H), 7.19 (d, J=8.09 Hz, 2H), 8.46 (dd, J=2.57, 8.46 Hz, 1H), 8.63 (d, J=2.57 Hz, 1H); EI-MS m/z (rel abundance) (M⁺, 100%).

### B31. General Method for Substituted Aniline Formation via Nitroarene Formation Through Nucleophilic Aromatic Substitution, Followed by Reduction

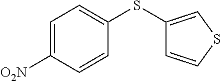

Step 1. 4-(3-Thienylthio)-1-nitrobenzene: To a solution of 4-nitrothiophenol (80% pure; 1.2 g, 6.1 mmol), 3-bromothiophene (1.0 g, 6.1 mmol) and copper(II) oxide (0.5 g, 3.7 mmol) in anhydrous DMF (20 mL) was added KOH (0.3 g, 6.1 mmol), and the resulting mixture was heated at 130° C. with stirring for 42 h and then allowed to cool to room temp. The reaction mixture was then poured into a mixture of ice and a 6N HCl solution (200 mL) and the resulting aqueous mixture was extracted with EtOAc (3×100 mL). The combined organic layers were sequentially washed with a 1M NaOH solution (2×100 mL) and a saturated NaCl solution (2×100 mL), dried (MgSO₄), and concentrated in vacuo. The residual oil was purified by MPLC (silica gel; gradient from 10% EtOAc/90% hexane to 5% EtOAc/95% hexane) to afford of the desired product (0.5 g, 34%). GC-MS m/z 237 (M⁺).

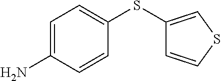

Step 2. 4-(3-Thienylthio)aniline: 4-(3-Thienylthio)-1-nitrobenzene was reduced to the aniline in a manlier analogous to that described in Method B1.

B3j. General Method for Substituted Aniline Formation via Nitroarene Formation. Through Nucleophilic Aromatic Substitution, Followed by Reduction

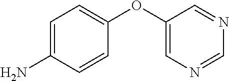

4-(5-Pyrimininyloxy)aniline: 4-Aminophenol (1.0 g, 9.2 mmol) was dissolved in DMF (20 mL) then 5-bromopyrimidine (1.46 g, 9.2 mmol) and K₂CO₃ (1.9 g, 13.7 mmol) were added. The mixture was heated to 100° C. for 18 h and at 130° C. for 48 h at which GC-MS analysis indicated some remaining starting material. The reaction mixture was cooled to room temp. and diluted with water (50 mL). The resulting solution was extracted with EtOAc (100 mL). The organic layer was washed with a saturated NaCl solution (2×50 mL), dried (MgSO₄), and concentrated in vacuo. The residular solids were purified by MPLC (50% EtOAc/50% hexanes) to give the desired amine (0.650 g, 38%).

B3k. General Method for Substituted Aniline Formation via Nitroarene Formation Through Nucleophilic Aromatic Substitution, Followed by Reduction

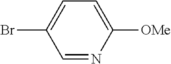

Step 1. 5-Bromo-2-methoxypyridine: A mixture of 2,5-dibromopyridine (5.5 g, 23.2 mmol) and NaOMe (3.76 g, 69.6 mmol) in MeOH (60 mL) was heated at 70° C. in a sealed reaction vessel for 42 h, then allowed to cool to room temp. The reaction mixture was treated with, water (50 mL) and extracted with EtOAc (2×100 mL). The combined organic layers were dried (Na₂SO₄) and concentrated under reduced pressure to give a pale yellow, volatile oil (4.1 g, 95% yield): TLC (10% EtOAc 190% hexane) R_{f} 0.57.

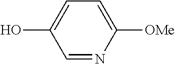

Step 2. 5-Hydroxy-2-methoxypyridine: To a stirred solution of 5-bromo-2-methoxypyridine (8.9 g, 47.9 mmol) in THF (175 mL) at −78° C. was added an n-butyllithium solution (2.5 M in hexane; 28.7 mL, 71.8 mmol) dropwise and the resulting mixture was allowed to stir at −78° C. for 45 min. Trimethyl borate (7.06 mL, 62.2 mmol) was added via syringe and the resulting mixture was stirred for an additional 2 h. The bright orange reaction mixture was warmed to 0° C. and was treated with a mixture of a 3 N NaOH solution (25 mL, 71.77 mmol) and a hydrogen peroxide solution (30%; approx. 50 mL). The resulting yellow and slightly turbid reaction mixture was warmed to room temp. for 30 min and then heated to the reflux temp. for 1 h. The reaction mixture was then allowed to cool to room temp. The aqueous layer was neutralized with a 1N HCl solution then extracted with Et₂O (2×100 mL). The combined organic layers were dried (Na₂SO₄) and concentrated under reduced pressure to give a viscous yellow oil (3.5 g, 60%).

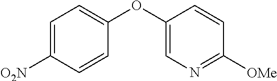

Step 3. 4-(5-(2-Methoxy)pyridyl)oxy-1-nitrobenzene: To a stirred slurry of NaH (97%, 1.0 g, 42 mmol) in anti DMF (100 mL) was added a solution of 5-hydroxy-2-methoxypyridine (3.5 g, 28 mmol) in DMF (100 mL). The resulting mixture was allowed to stir at room temp. for 1 h, 4-fluoronitrobenzene (3 mL, 28 mmol) was added via syringe. The reaction mixture was heated to 95° C. overnight, then treated with water (25 mL) and extracted with EtOAc (2×75 mL). The organic layer was dried (MgSO₄) and concentrated under reduced pressure. The residual brown oil was crystallized EtOAc/hexane) to afford yellow crystals (5.23 g, 75%).

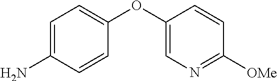

Step 4. 4-(5-(2-Methoxy)pyridyl)oxyaniline: 4-(5-(2-Methoxy)pyridyl)oxy-1-nitrobenzene was reduced to the aniline in a manner analogous to that described in Method B3d, Step 2.

B4a. General Method for Substituted Aniline Synthesis via Nucleophilic Aromatic Substitution Using a Halopyridine

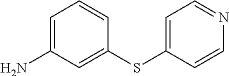

3-(4-Pyridinylthio)aniline: To a solution of 3-aminothiophenol (3.8 mL, 34 mmoles) in anh DMF (90 mL) was added 4-chloropyridine hydrochloride (5.4 g, 35.6 mmoles) followed by K₂CO₃ (16.7 g, 121 mmoles). The reaction mixture was stirred at room temp. for 1.5 h, then diluted with EtOAc (100 mL) and water (100 mL), The aqueous layer was back-extracted with EtOAc (2×100 mL). The combined organic layers were washed with a saturated NaCl solution (100 mL), dried (MgSO₄), and concentrated under reduced pressure. The residue was filtered through a pad of silica (gradient from 50% EtOAc/50% hexane to 70% EtOAc/30% hexane) and the resulting material was triturated with a Et₂O/hexane solution to afford the desired product (4.6 g, 66%): TLC (100% ethyl acetate) R_{f} 0.29; ¹H-NMR (DMSO-d₆) δ 5.41 (s, 2H), 6.64-6.74 (m, 3H), 7.01 (d, J=4.8, 2H), 7.14 (t, J=7.8 Hz, 1H), 8.32 (d, 2H).

2B4b. General Method for Substituted Aniline Synthesis via Nucleophilic Aromatic Substitution using a Halopyridine

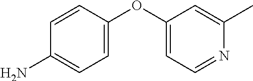

4-(2-Methyl-4-pyridinyloxy)aniline: To a solution of 4-aminophenol (3.6 g, 32.8 mmol) and 4-chloropicoline (5.0 g, 39.3 mmol) in anh DMPU (50 mL) was added potassium tert-butoxide (7.4 g, 65.6 mmol) in one portion. The reaction mixture was heated at 100° C. with stirring for 18 h, then was allowed to cool to room temp. The resulting mixture was poured into water (200 mL) and extracted with EtOAc (3×150 mL). The combined extracts were sequentially washed with water (3×100 mL) and a saturated NaCl solution (2×100 mL), dried (Na₂SO₄), and concentrated in vacuo. The resulting oil was purified by flash chromatography (50% EtOAc/50% hexane) to afford the desired product as a yellow oil (0.7 g, 9%): CI-MS m/z 201 ((M+H)⁺).

B4c. General Method for Substituted Aniline Synthesis via Nucleophilic Aromatic Substitution using a Halopyridine

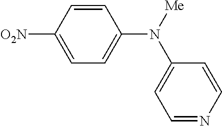

Step 1. Methyl(4-nitrophenyl)-4-pyridylamine: To a suspension of N-methyl-4-nitroaniline (2.0 g, 13.2 mmol) and K₂CO₃ (7.2 g, 52.2 mmol) in DMPU (30 mL) was added 4-chloropyridine hydrochloride (2.36 g, 15.77 mmol). The reaction mixture was heated at 90° C. for 20 h, then cooled to room temperature. The resulting mixture was diluted with water (100 mL) and extracted with EtOAc (100 mL). The organic layer was washed with water (100 mL), dried (Na₂SO₄) and concentrated under reduced pressure. The residue was purified by column chromatography (silica gel, gradient from 80% EtOAc/20% hexanes to 100% EtOAc) to afford methyl(4-nitrophenyl)-4-pyridylamine (0.42 g)

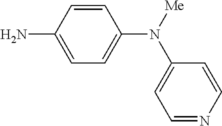

Step 2. Methyl(4-aminophenyl)-4-pyridylamine: Methyl(4-nitrophenyl)-4-pyridylamine was reduced in a manner analogous to that described in Method B1.

### B5. General Method of Substituted Aniline Synthesis via Phenol Alkylation Followed by Reduction of a Nitroarene

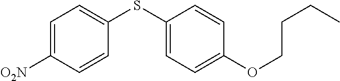

Step 1. 4-(4-Butoxyphenyl)thio-1-nitrobenzene: To a solution of 4-(4-nitrophenyl-thio)phenol (1.50 g, 6.07 mmol) in arch DMF (75 ml) at 0° C. was added NaH (60% in mineral oil, 0.267 g, 6.67 mmol). The brown suspension was stirred at 0° C. until gas evolution stopped (15 min), then a solution of iodobutane (1.12 g, 0.690 ml, 6.07 mmol) in anh DMF (20 mL) was added dropwise over 15 min at 0° C. The reaction was stirred at room temp. for 18 h at which time TLC indicated the presence of unreacted phenol, and additional iodobutane (56 mg, 0.035 mL, 0.303 mmol, 0.05 equiv) and NaH (13 mg, 0.334 mmol) were added. The reaction was stirred an additional 6 h room temp., then was quenched by the addition of water (400 mL) The resulting mixture was extracted with Et₂O (2×500 mL). The combibed organics were washed with water (2×400 mL), dried (MgSO₄), and concentrated under reduced pressure to give a clear yellow oil, which was purified by silica gel chromatography (gradient from 20% EtOAc/80% hexane to 50% EtOAc/50% hexane) to give the product as a yellow solid (1.24 g, 67%): TLC (20% EtOAc/80% hexane) 0.75; ¹H-NMR (DMSO-d₆) δ 0.92 (t, 7.5 Hz, 3H), 1.42 (app hex, J=7.5 Hz, 2H), 1.70 (m, 2H), 4.01 (t, J=6.6 Hz, 2H), 7.08 (d, J=8.7 Hz, 2H), 7.17 (d, Hz, 2H), 7.51 (d, 8.7 Hz, 2H), 8.09 (d, J=9 Hz, 2H),

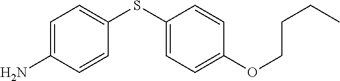

Step 2. 4-(4-Butoxyphenyl)thioaniline: 4-(4-Butoxyphenyl)thio-1-nitrobenzene was reduced to the aniline in a manner analagous to that used in the preparation of 3-(trifluoromethyl)-4-(4-pyridinylthio)aniline (Method B3b, Step 2): TLC (33% EtOAc/77% hexane) R_{f} 0.38.

### B6. General Method for Synthesis of Substituted Anilines by the Acylation of Diaminoarenes

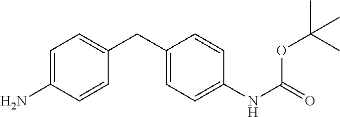

4-(4-tert-Butoxycarbamoylbenzyl)aniline: To a solution of 4,4′-methylenedianiline (3.00 g, 15.1 mmol) in anh THF (50 mL) at room temp was added a solution of di-tert-butyl dicarbonate (3.30 g, 15.1 mmol) in anh THF (10 mL). The reaction mixture was heated at the reflux temp. for 3 h, at which time TLC indicated the presence of unreacted methylenedianiline. Additional di-tert-butyl dicarbonate (0.664 g, 3.03 mmol, 0.02 equiv) was added and the reaction stirred at the reflux temp. for 16 h. The resulting mixture was diluted with Et₂O (200 mL), sequentially washed with a saturated NaHCO₃ solution (100 ml), water (100 mL) and a saturated NaCl solution (50 mL), dried (MgSO₄), and concentrated under reduced pressure. The resulting white solid was purified by silica gel chromatography (gradient from 33% EtOAc/67% hexane to 50% EtOAc/50% hexane) to afford the desired product as a white solid (2.09 g, 46%): TLC (50% EtOAc/50% hexane) R_{f} 0.45; ¹H-NMR (DMSO-d₆) δ 1.43 (s, 9H), 3.63 (s, 2H), 4.85 (br s, 2H), 6.44 (d, J=8.4 Hz, 2H), 6.80 (d, J=8.1 Hz, 2H), 7.00 (d, J=8.4 Hz, 2H), 7.28 (d, J=8.1 Hz, 2H), 9.18 (br s, 1H); FAB-MS m/z 298 (M⁺).

### 1B7. General Method for the Synthesis of Aryl Amines via Electrophilic Nitration Followed by Reduction

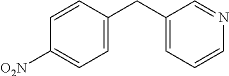

Step 1. 3-(4-Nitrobenzyl)pyridine: A solution of 3-benzylpyridine (4.0 g, 23.6 mmol) and 70% nitric acid (30 mL) was heated overnight at 50° C. The resulting mixture was allowed to cool to room temp. then poured into ice water (350 mL). The aqueous mixture then made basic with a 1N NaOH solution, then extracted with Et₂O (4×100 mL). The combined extracts were sequentially washed with water (3×100 mL) and a saturated NaCl solution (2×100 mL), dried (Na₂SO₄), and concentrated in vacuo. The residual oil was purified by MPLC (silica gel; 50% EtOAc/50% hexane) then recrystallization (EtOAc/hexane) to afford the desired product (1.0 g, 22%): GC-MS m/z 214 (M⁺).

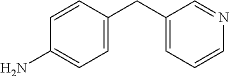

Step 2. 3-(4-Pyridinyl)methylaniline: 3-(4-Nitrobenzyl)pyridine was reduced to the aniline in a manner analogous to that described in Method B1.

B8. General Method for Synthesis of Aryl Amines via Substitution with Nitrobenzyl Halides Followed by Reduction

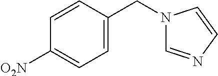

Step 1. 4-(1-imidazolylmethyl)-1-nitrobenzene: To a solution of imidazole (0.5 g, 7.3 mmol) and 4-nitrobenzyl bromide (1.6 g, 7.3 mmol) in anh acetonitrile (30 mL) was added K₂CO₃ (1.0 g, 7.3 mmol). The resulting mixture was stirred at room temp. for 18 h and then poured into water (200 mL) and the resulting aqueous solution was extracted with EtOAc (3×50 mL). The combined organic layers were sequentially washed with water (3×50 mL) and a saturated NaCl solution (2×50 mL), dried (MgSO₄), and concentrated in vacuo. The residual oil was purified by MPLC (silica gel; 25% EtOAc/75% hexane) to afford the desired product (1.0 g, 91%): EI-MS m/z 203 (M⁺).

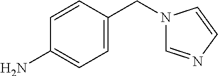

Step 2. 4-(1-Imidazolylmethyl)aniline: 4-(1-Imidazolylmethyl)-1-nitrobenzene was reduced to the aniline in a manner analogous to that described in Method B2.

### 1B9. Formation of Substituted Hydroxymethylanilines by Oxidation of Nitrobenzyl Compounds Followed by Reduction

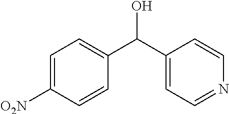

Step 1. 4-(1-Hydroxy-1-(4-pyridyl)methyl-1-nitrobenzene: To a stirred solution of 3-(4-nitrobenzyl)pyridine (6.0 g, 28 mmol) in CH₂Cl₂ (90 mL) was added m-CPBA (5.80 g, 33.6 mmol) at 10° C., and the mixture was stirred at room temp. overnight. The reaction mixture was successively washed with a 10% NaHSO₃ solution (50 mL), a saturated K₂CO₃ solution (50 mL) and a saturated NaCl solution (50 mL), dried (MgSO₄) and concentrated under reduced pressure. The resulting yellow solid (2.68 g) was dissolved in anh acetic anhydride (30 mL) and heated at the reflux temperature overnight. The mixture was concentrated under reduced pressure. The residue was dissolved in MeOH (25 mL) and treated with a 20% aqueous NH₃ solution (30 mL). The mixture was stirred at room temp. for 1 h, then was concentrated under reduced pressure. The residue was poured into a mixture of water (50 mL) and CH₂Cl₂ (50 mL). The organic layer was dried (MgSO₄), concentrated under reduced pressure, and purified by column chromatography (80% EtOAc/20% hexane) to afford the desired product as a white solid. (0.53 g, 8%): mp 110-118° C.; TLC (80% EtOAc/20% hexane) R_{f} 0.12; FAB-MS m/z 367 ((M+H)⁺, 100%).

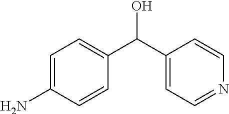

Step 2. 4-(1-Hydroxy-1-(4-pyridyl)methylaniline: 4-(1-Hydroxy-1-(4-pyridyl)-methyl-1-nitrobenzene was reduced to the aniline in a manner analogous to that described in Method B3d, Step 2.

B10. Formation of 2-(N-methylcarbamoyl)pyridines via the Menisci reaction

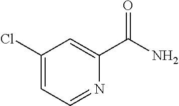

Step 1. 2-(N-methylcarbamoyl)-4-chloropyridine. (Caution: this is a highly hazardous, potentially explosive reaction.) To a solution of 4-chloropyridine (10.0 g) in N-methylformamide (250 mL) under argon at ambient temp was added cone. H₂SO₄ (3.55 mL) (exotherm). To this was added H₂O, (17 mL, 30% wt in H2O) followed by FeSO₄.7H2O (0.55 g) to produce an exotherm. The reaction was stirred in the dark at ambient temp for 1 h then was heated slowly over 4 h at 45° C. When bubbling subsided, the reaction was heated at 60° C. for 16 h. The opaque brown solution was diluted with H2O (700 mL) followed by a 10% NaOH solution (250 mL). The aqueous mixture was extracted with EtOAc (3×500 mL) and the organic layers were washed separately with a saturated NaCl solution (3×150 mlL. The combined organics were dried (MgSO₄) and filtered through a pad of silica gel eluting with EtOAc. The solvent was removed in vacuo and the brown residue was purified by silica gel chromatography (gradient from 50% EtOAc/50% hexane to 80% EtOAc/20% hexane). The resulting yellow oil crystallized at 0° C. over 72 h to give 2-(N-methylcarbamoyl)-4-chloropyridine in yield (0.61 g, 5.3%): TLC (50% EtOAc/50% hexane) R_{f} 0.50; MS; NMR (CDCl₃): d 8.44 (d, 1H, J=5.1 Hz, CHN), 8.21 (s, 1H, CHCCO), 7.96 (b s, 1H, NH), 7.43 (dd, 1H, J=2.4, 5.4 Hz, ClCHCN), 3.04 (d, 3H, J=5.1 Hz, methyl); CI-MS m/z 171 ((M+H)+).

### B11. General Method for the Synthesis of ω-Sulfonylphenyl Anilines

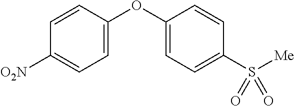

Step 1. 4-(4-Methylsulfonylphenoxy)-1-nitrobenzene: To a solution of 4-(4-methylthiophenoxy)-1-nitrobenzene (2 g, 7.66 mmol) in CH₂Cl₂ (75 mL) at 0° C. was slowly added in CPBA (57-86%, 4 g), and the reaction mixture was stirred at room temperature for 5 h. The reaction mixture was treated with a 1 N NaOH solution (25 mL). The organic layer was sequentially washed with a 1N NaOH solution (25 mL), water (25 mL) and a saturated NaCl solution (25 mL), dried (MgSO₄), and concentrated under reduced pressure to give 4-(4-methylsulfonylphenoxy)-1-nitrobenzene as a solid (2.1 g).

Step 2. 4-(4-Methylsulfonylphenoxy)-1-aniline: 4-(4-Methylsulfonylphenoxy)-1-nitrobenzene was reduced to the aniline in a manner analogous to that described in Method B3d, step 2.

B12. General Method for Synthesis of ω-Alkoxy-ω-carboxyphenyl Anilines

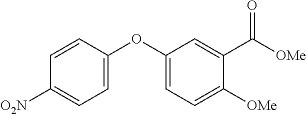

Step 1. 4-(3-Methoxycarbonyl-4-methoxyphenoxy)-1-nitrobenzene: To a solution of -(3-carboxy-4-hydroxyphenoxy)-1-nitrobenzene (prepared in a manner analogous to that described in Method B3a, step 1, 12 mmol) in acetone (50 mL) was added K₂C0 (5 g) and dimethyl sulfate (3.5 mL). The resulting mixture was heated at the reflux temperature overnight, then cooled to room temperature and filtered through a pad of Celite®. The resulting solution was concentrated under reduced pressure, absorbed onto silica gel, and purified by column chromatography (50% EtOAc/50% hexane) to give 4-(3-methoxycarbonyl-4-methoxyphenoxy)-1-nitrobenzene as a yellow powder (3 g): mp 115 118° C.

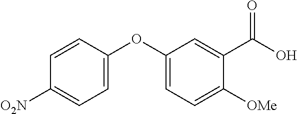

Step 2. 4-(3-Carboxy-4-methoxyphenoxy)-1-nitrobenzene: A mixture of 4-(3-methoxycarbonyl-4-methoxyphenoxy)-1-nitrobenzene (1.2 g), KOH (0.33 g), and water (5 mL) in MeOH (45 mL) was stirred at room temperature overnight and then heated at the reflux temperature for 4 h. The resulting mixture was cooled to room temperature and concentrated under reduced pressure. The residue was dissolved in water (50 mL), and the aqueous mixture was made acidic with a 1N HCl solution. The resulting mixture was extracted with EtOAc (50 mL). The organic layer was dried (MgSO₄) and concentrated under reduced pressure to give 4-(3-carboxy-4-methoxyphenoxy)-1-nitrobenzene (1.04 g).

### C. General Methods of Urea Formation

C1a. Reaction of a Heterocyclic Amine with an Isocyanate

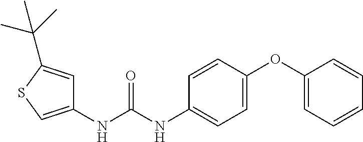

N-(5-tert-Butyl-3-thienyl)-N′-(4-phenoxyphenyl)urea: To a solution of 5-tert-butyl-3-thiophene-ammonium chloride (prepared as described in Method A4b; 7.28 g, 46.9 mmol, 1.0 equiv) in anh DMF (80 mL) was added 4-phenoxyphenyl isocyanate (8.92 g, 42.21 mmol, 0.9 equiv) in one portion. The resulting solution was stirred at 50-60° C. overnight, then diluted with EtOAc (300 mL). The resulting solution was sequentially washed with H₂O (200 mL), a 1 N HCl solution (50 mL) and a saturated NaCl solution (50 mL), dried (Na₂SO₄), and concentrated under reduced pressure. The resulting off-white solid was recrystallized (EtOAc/hexane) to give a white solid (13.7 g, 88%), which was contaminated with approximately 5% of bis(4-phenoxyphenyl)urea, A portion of this material (4.67 g) was purified by flash chromatography (9% EtOAc/27% CH₂Cl₂/64% cyclohexane) to afforded the desired product as a white solid (3.17 g).

C1b. Reaction of a Heterocyclic Amine with an Isocyanate

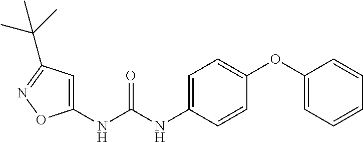

N-(3-tert-Butyl-5-isoxazolyl)-N′-(4-phenoxyphenyl)urea: To a solution of 5-amino-3-tert-butylisoxazole (8.93 g, 63.7 mmol, 1 eq.) in CH₂Cl₂ (60 mL) was added 4-phenyloxyphenyl isocyanate (15.47 g, 73.3 mmol, 1.15 eq.) dropwise. The mixture was heated at the reflux temp. for 2 days, eventually adding additional CH₂Cl₂ (80 mL). The resulting mixture was poured into water (500 mL) and extracted with Et₂O (3×200 mL). The organic layer was dried (MgSO₄) then concentrated under reduced pressure. The residue was recrystallized (EtOAc) to give the desired product (15.7 g, 70%): mp 182-184° C.; TLC (5% acetone/95% acetone) R_{f} 0.27; ¹H-NMR (DMSO-d₆) δ 1.23 (s, 9H), 6.02 (s, 1H), 6.97 (dd, J=0.2, 8.8 Hz, 2H), 6.93 (d, J=8.8 Hz, 2H), 7.08 (t, J=7.4 Hz, 1H), 7.34 (m, 2H), 7.45 (dd, J=2.2, 6.6 Hz, 2H), 8.80 (s, 1H), 10.04 (s, 1H); FAB-MS m/z (rel abundance) 352 ((M+H)⁺, 70%).

C1c. Reaction of a Heterocyclic Amine with an Isocyanate

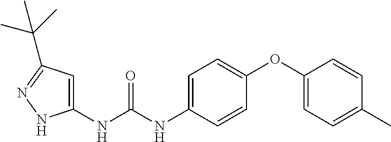

N-(3-tert-Butyl-5-pyrazolyl)-N′-(4-(4-methylphenyl)oxyphenyl)urea: A solution of 5-amino-3-tert-butylpyrazole (0.139 g, 1.0 mmol, 1.0 equiv) and 4-(4-methylphenoxy)phenyl isocyanate (0.225 g, 1.0 mmol 1.0 equiv) in toluene (10 mL) was heated at the reflux temp. overnight. The resulting mixture was cooled to room temp and quenched with MeOH (a few mL). After stirring for 30 min, the mixture was concentrated under reduced pressure. The residue was purified by prep. HPLC (silica, 50% EtOAc/50% hexane) to give the desired product (0.121 g, 33%): mp 204° C.; TLC (5% acetone/95% CH₂Cl₂) R_{f} 0.92; ¹H-NMR (DMSO-d) δ 1.22 (s, 9H), 2.24 (s, 3H), 5.92 (s, 1H), 6.83 (d, J=8.4 Hz, 2H), 6.90 (d, J=8.8 Hz, 2H), 7.13 (d, J=8.4 Hz, 2H), 7.40 (d, J=8.8 Hz, 2H), 8.85 (s, 1H), 9.20 (br s, 1H), 11.94 (br s, 1H); EI-MS m/z 364 (M⁺).

C1d. Reaction of a Heterocyclic Amine with an Isocyanate

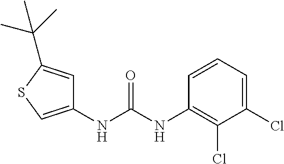

N-(5-tert-Butyl-3-thienyl)-N′-(2,3-dichlorophenyl)urea: Pyridine (0.163 mL, 2.02 mmol) was added to a slurry of 5-tert-butylthiopheneammonium chloride (Method A4c; 0.30 g, 1.56 mmol) and 2,3-dichlorophenyl isocyanate (0.32 mL, 2.02 mmol) in CH₂Cl₂ (10 mL) to clarify the mixture and the resulting solution was stirred at room temp. overnight. The reaction mixture was then concentrated under reduced pressure and the residue was separated between EtOAc (15 mL) and water (15 mL). The organic layer was sequentially washed with a saturated NaHCO₃ solution (15 mL), a 1N:HCl solution (15 mL) and a saturated NaCl solution (15 mL), dried (Na₂SO₄), and concentrated under reduced pressure. A portion of the residue was by preparative HPLC (C-18 column; 60% acetonitrile/40% water/0.05% TFA) to give the desired urea (0.180 g, 34%): mp 169-170° C.; TLC (20% EtOAc/80% hexane) R_{f} 0.57; ¹H-NMR (DMSO-d₆) δ 1.31 (s, 9H), 6.79 (s, 1H), 7.03 (s, 1H), 7.24-7.33 (an, 2H), 8.16 (dd, J=1.84, 7.72 Hz, 1H), 8.35 (s, 1H), 9.60 (s, 1H); ¹³C-NMR (DMSO-d₆) δ 31.9 (3C), 34.0, 103.4, 116.1, 119.3, 120.0, 123.4, 128.1, 131.6, 135.6, 138.1, 151.7, 155.2; FAB-MS m/z (rel abundance) 343 ((M+H)⁺, 83%), 345 ((M+H+2)⁺, 56%), 347 ((M+H+4)⁺, 12%).

C1e, Reaction of a Heterocyclic Amine with an Isocyanate

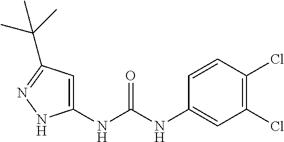

N-(3-tert-Butyl-5-pyrazolyl)-N′-(3,4-dichlorophenyl)urea: A solution of 5-amino-3-tert-butyl-N′-(tert-butoxycarbonyl)pyrazole (Method A5; 0.150 g, 0.63 mmol) and 3,4-dichlorophenyl isocyanate (0.118 g, 0.63 mmol) were in toluene (3.1 mL) was stirred at 55° C. for 2 d. The toluene was removed in vacuo and the solid was redissolved in a mixture of CH₂Cl₂ (3 mL) and TFA (1.5 mL). After 30 min, the solvent was removed in vacuo and the residue was taken up in EtOAc (10 mL). The resulting mixture was sequentially washed with a saturated NaHCO₃ solution (10 mL) and a NaCl solution (5 mL), dried (Na₂SO₄), and concentrated in vacuo. The residue was purified by flash chromatography (gradient from 40% EtOAc/60% hexane to 55% EtOAc/5% hexane) to give the desired product (0.102 g, 48%): mp 182.484° C.; TLC (40% EtOAc/60% hexane) R_{f} 0.05, FAB-MS m/z 327 ((M+H)⁺).

C2a. Reaction of a Heterocyclic Amine with Phosgene to Form an isocyanate, then Reaction with Substituted Aniline

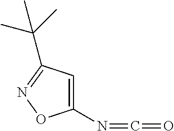

Step 1. 3-tert-Butyl-5-isoxazolyl Isocyanate: To a solution of phosgene (20% in toluene, 1.13 mL, 2.18 mmol) ins CH₂Cl₂ (20 mL) at 0° C. was added anh. pyridine (0.176 mL, 2.18 mmol), followed by 5-amino-3-tert-butylisoxazole (0.305 g, 2.18 mmol). The resulting solution was allowed to warm to room temp. over 1 h, and then was concentrated under reduced pressure. The solid residue dried in vacuo for 0.5 h.

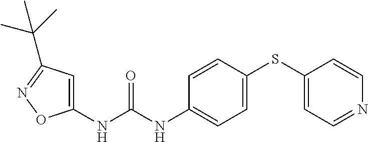

Step 2. N-(3-tert-Butyl-5-isoxazolyl)-N′-(4-(4-pyridinylthio)phenyl)urea: The crude 3-tert-butyl-5-isoxazolyl isocyanate was suspended in anh toluene (10 mL) and 4-(4-pyridinylthio)aniline (0.200 g, 0.989 mmol) was rapidly added. The suspension was stirred at 80° C. for 2 h then cooled to room temp. and diluted with an EtOAc/CH₂Cl₂ solution (4:1, 125 mL). The organic layer was washed with water (100 mL) and a saturated NaCl solution (50 mL), dried (MgSO₄), and concentrated under reduced pressure. The resulting yellow oil was purified by column chromatography (silica gel, gradient from 2% MeOH/98% CH₂Cl₂ to 4% MeOH/6%

CH₂Cl₂) to afford a foam, which was triturated (Et_{z}0/hexane) in combination with sonication to give the product as a white powder (0.18 g, 49%): TLC (5% MeOH/95% CH₂Cl₃) R_{f} 0.21; ¹H-NMR (DMSO-d₅) δ 1.23 (s, 9H), 6.06 (s, 1H), 6.95 (d, J=5 Hz, 2H), 7.51 (d, J=8 Hz, 2H), 7.62 (d, j=8 Hz, 2H), 8.32 (d, J=5 Hz, 2H), 9.13 (s, 1H), 10.19 (s, 1H); FAB-MS m/z 369 ((M+H)⁺).

C2b. Reaction of a Heterocyclic Amine with Phosgene to Form an Isocyanate Followed by Reaction with Substituted Aniline

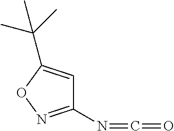

Step 1. 5-tert-Butyl-3-isoxazolyl Isocyanate: To a solution of phosgene (148 mL, 1.93 M in toluene, 285 mmol) in anhydrous CH₂Cl₂ (1 L) was added 3-amino-5-tert-butylisoxazole (10.0 g, 71 mmol) followed by pyridine (46 mL, 569 mmol). The mixture was allowed to warm to room temp and stirred overnight (ca. 16 h), then mixture was concentrated in vacuo. The residue was dissolved in anh. THF (350 mL) and stirred for 10 min. The orange precipitate (pyridinium hydrochloride) was removed and the isocyanate-containing filtrate (approximately 0.2 M in THF) was used as a stock solution: GC-MS (aliquot obtained prior to concentration) m/z 166 (M⁺).

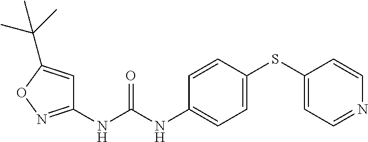

Step 2. N-(5-tert-Butyl-3-isoxazolyl)-N′-(4-(4-pyridinylthio)phenyl)urea: To a solution of 5-tert-butyl-3-isoxazolyl isocyanate (247 mL, 0.2 M in THF, 49.4 mmol) was added 4-(4-pyridinylthio)aniline (5 g, 24.72 mmol), followed by THY (50 mL) then pyridine (4.0 mL, 49 mmol) to neutralize any residual acid. The mixture was stirred overnight (ca. 18 h) at room temp. Then diluted with EtOAc (300 mL). The organic layer was washed successively with a saturated NaCl solution (100 mL), a saturated NaHCO₃ solution (100 mL), and a saturated NaCl solution (100 mL), dried (MgSO₄), and concentrated in vacuo. The resulting material was purified by MPLC (2×300 g silica gel, 30% EtOAc/70% hexane) to afford the desired product as a white solid (8.24 g, 90%): mp 178-179° C.; ¹H-NMR (DMSO-d₆) δ 1.28 (s, 9H), 6.51 (s, 1H), 6.96 (d, J=6.25 Hz, 2H), 7.52 (d, J=8.82 Hz, 2H), 7.62 (d, J=8.83 Hz, 2H), 8.33 (d, J=6.25 Hz, 2H), 9.10 (s, 1H), 9.61 (s, 1H); EI-MS m/z 368 (M⁺).

C2c. Reaction of a Heterocyclic Amine with Phosgene to Form an Isocyanate Followed by Reaction with Substituted Aniline

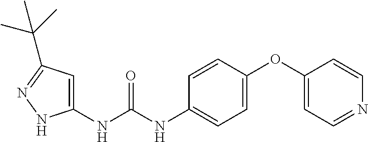

N-(3-tert-Butyl-5-pyrazolyl)-N′-(4-(4-pyridinyloxy)phenyl)urea: To a solution of phosgene (1.9M in toluene, 6.8 mL) in anhydrous CH₂Cl₂ (13 mL) at 0° C. was slowly added pyridine (0.105 mL) was added slowly over a 5 min, then 4-(4-pyridinyloxy)aniline (0.250 g, 1.3 mmol) was added in one aliquot causing a transient yellow color to appear. The solution was stirred at 0° C. for 1 h, then was allowed to warm to room temp. over 1 h. The resulting solution was concentrated in vacuo then the white solid was suspended in toluene (7 mL). To this slurry, 5-amino-3-tert-butyl-N¹-(tert-butoxycarbonyl)pyrazole (0.160 g, 0.67 mmol) was added in one aliquot and the reaction mixture was heated at 70° C. for 12 h forming a white precipitate. The solids were dissolved in a 1N HCl solution and allowed to stir at room temp. for 1 h to form a new precipitate. The white solid was washed (50% Et₂O/50% pet. ether) to afford the desired urea (0.139 g, 59%): mp >228° C. dec; TLC (10% MeOH/90% CHCl₃) R_{f} 0.239; ¹H-NMR (DMSO-d₆) δ 1.24 (s, 9H), 5.97 (s, 1H), 6.88 (d, J=6.25 Hz, 2H), 7.10 (d, J=8.82 Hz, 2H), 7.53 (d, J=9.2 Hz, 2H), 8.43 (d, J=6.25 Hz, 2H), 8.92 (br s, 1H), 9.25 (br s, 1H), 12.00 (br s, 1H); EI-MS m/z rel abundance 351 (M⁺, 24%).

C3a. Reaction of a Heterocyclic Amine with N,N′-Carbonyldiimidazole Followed by Reaction with a Substituted Aniline

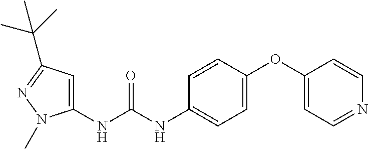

N-(3-tert-Butyl-1-methyl-5-pyrazolyD-N′-(4-(4-pyridinyloxy)phenyl)urea: To a solution of 5-ammo-3-tert-butyl-1-methylpyrazole (189 g, 1.24 mol) in anh. CH₂Cl₂ (2.3 L) was added N,N′-carbonyldiimidazole (214 g, 1.32 mol) in one portion. The mixture was allowed to stir at ambient temperature for 5 h before adding 4-(4-pyridinyloxy)aniline. The reaction mixture was heated to 36° C. for 16 h. The resulting mixture was cooled to room temp, diluted with EtOAc (2 L) and washed with H₂O (8 L) and a saturated NaCl solution (4 L). The organic layer was dried (Na₂SO₄) and concentrated in vacuo. The residue was purified by crystallization (44.4% EtOAc/44.4% Et₂O111.2% hexane, 2.5 L) to afford the desired urea as a white solid (230 g, 51%): mp 149-152° C.; ¹H NMR (DMSO-d₆) δ 1.18 (s, 9H), 3.57 (s, 3H), 6.02 (s, 1H), 6.85 (d, J=6.0 Hz, 2H), 7.08 (d, J=9.0 Hz, 2H), 7.52 (d, J=9.0 Hz, 2H), 8.40 (d, J=6.0 Hz, 2H), 8.46 (s, 1H), 8.97 (s, 1H); FAB-LSIMS m/z 366 ((M+H)⁺).

C3b. Reaction of a Heterocyclic Amine with N,N′-Carbonyldiimidazole Followed by Reaction with a Substituted Aniline

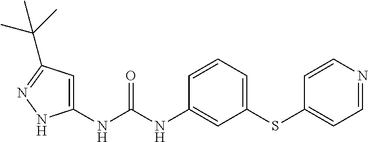

N-(3-tert-Butyl-5-pyrazolyl)-N′-(3-(4-pyridinylthio)phenyl)urea: To a solution of 5-amino-3-tert-butyl-N¹-(tert-butoxycarbonyl)pyrazole (0.282 g, 1.18 mmol) in CH₂Cl₂ (1.2 mL) was added N,N′-carbonyldiimidazole (0.200 g, 1.24 mmol) and the mixture was allowed to stir at room temp. for 1 day. 3-(4-Pyridinylthio)aniline (0.239 g, 1.18 mmol) was added to the reaction solution in one aliquot and the resulting mixture was allowed to stir at room temp. for 1 day. Then resulting solution was treated with a 10% citric acid solution (2 mL) and was allowed to stir for 4 h. The organic layer was extracted with EtOAc (3×15 mL), dried (MgSO₄), and concentrated in vacuo. The residue was diluted with CH₂Cl₂ (5 mL) and trifluoroacetic acid (2 mL) and the resulting solution was allowed to stir for 4 h. The trifluoroacetic reaction mixture was made basic with a saturated NaHCO₃ solution, then extracted with CH₂Cl₂ (3×15 mL). The combined organic layers were dried (MgSO₄) and concentrated in vacuo. The residue was purified by flash chromatography (5% MeOH/95% CH₂Cl₂). The resulting brown solid was triturated with sonication (50% Et₂O/50% pet. ether) to give the desired urea (0.122 g, 28%): mp >224° C. dec; TLC (5% MeOH/95% CHCl₃) R_{f} 0.067; ¹H-NMR (DMSO-d₅) δ 1.23 (s, 9H), 5.98 (s, 1H), 7.04 (dm, J=13.24 Hz, 214), 7.15-7.19 (m, 1H), 7.40-7.47 (m, 2H), 7.80-7.82 (m, 1H), 8.36 (dm, J=15.44 Hz, 2H), 8.96 (br s, 1H), 9.32 (br s, 1H), 11.97 (br s, 1H); FAB-MS m/z (rel abundance) 368 (M⁺, 100%).

C4a. Reaction of Substituted Aniline with N,N′-Carbonyldiimidazole Followed by Reaction with a Heterocyclic Amine

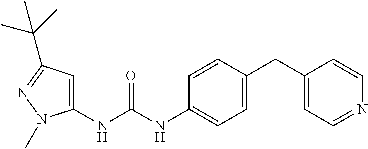

N-(3-tert-Butyl-1-methyl-5-pyrazolyl)-N′-(4-(4-pyridinylmethyl)phenyl)urea: To a solution of 4-(4-pyridinylmethyl)aniline (0.200 g, 1.08 mmol) in CH₂Cl₂ (10 mL) was added N,N′-carbonyldiimidazole (0.200 g, 1.23 mmol). The resulting mixture was stirred at room tempe for 1 h after which TLC analysis indicated no starting aniline. The reaction mixture was then treated with 5-amino-3-tert-butyl-1-methylpyrazole (0.165 g, 1.08 mmol) and stirred at 40-45° C. overnight. The reaction mixture was cooled to room temp and purified by column chromatography (gradient from 20% acetone/80% CH₂Cl₂ to 60% acetone/40% CH₂Cl₂) and the resulting solids were crystallized (Et₂O) to afford the desired urea (0.227 g, 58%): TLC (4% MeOH/96% CH₂Cl₂) R_{f} 0.15; ¹H-NMR (DMSO-d₆) δ 1.19 (s, 9H), 3.57 (s, 3H), 3.89 (s, 2H), 6.02 (s, 1H), 7.14 (d, J=8.4 Hz, 2H), 7.21 (d, J=6 Hz, 2H), 7.37 (d, J=8.4 Hz, 2H), 8.45-8.42 (m, 3H), 8.81 (s, 1H); FAB-MS m/z 364 (M+H)⁺).

C4b. Reaction of Substituted Aniline with N,N′-Carbonyldiimidazole Followed by Reaction with a Heterocyclic Amine

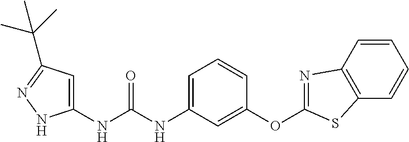

N-(3-tert-Butyl-5-pyrazolyl)-N′-(3-(2-benzothiazolyloxy)phenyl)urea: A solution of 3-(2-benzothiazolyloxy)aniline (0.24 g, 1.0 mmol, 1.0 equiv) and N,N′-carbonyldiimidazole (0.162 g, 1.0 mmol, 1.0 equiv) in toluene (10 mL) was stirred at room temp for 1 h. 5-Amino-3-tert-butylpyrazole (0.139 g, 1.0 mmol) was added and the resulting mixture was heated at the reflux temp, overnight. The resulting mixture was poured into water and extracted with CH₂Cl₂ (3×50 mL). The combined organic layers were concentrated under reduced pressure and dissolved in a minimal amount of CH₂Cl₂. Petroleum ether was added and resulting white precipitate was resubmitted to the crystallization protocol to afford the desired product (0.015 g, 4%): mp 110-111° C.; TLC (5% acetone/95% CH₂Cl₂) R_{f} 0.05; ¹H NMR (DMSO-d₆) δ 1.24 (s, 9H), 5.97 (s, 1H), 7.00-7.04 (m, 1H), 7.21-7.44 (m, 4H), 7.68 (d, J=5.5 Hz, 1H), 7.92 (d, J=7.7 Hz, 1H), 730 (s, 1H), 8.95 (s, 1H), 9.34 (br s, 1H), 11.98 (br s, 1H); EI-MS m/z 408 (M⁺).

C4c. Reaction of a Heterocyclic Amine with Phosgene to Form an Isocyanate Followed by Reaction with Substituted Aniline

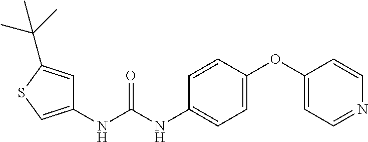

N-(5-tert-Butyl-3-thienyl)-N′-(4-(4-pyridinyloxy)phenyl)urea: To an ice cold solution phosgene (1.93M in toluene; 0.92 mL, 1.77 mmol) in CH₂Cl₂ (5 mL) was added a solution of 4-(4-pyridinyloxy)aniline (0.30 g, 1.61 mmol) and pyridine (0.255 g, 3.22 mmol) in CH₂Cl₂ (5 mL). The resulting mixture was allowed to warm to room temp. and was stirred for 1 h, then was concentrated under reduced pressure. The residue was dissolved in CH₂Cl₂ (5 mL), then treated with 5-tert-butylthiopheneammonium chloride (Method A4c; 0.206 g, 1.07 mmol), followed by pyridine (0.5 mL). The resulting mixture was stirred at room temp for 1 h, then treated with 2-(dimethylamino)ethylamine (1 mL), followed by stirring at room temp an additional 30 min. The reaction mixture was then diluted with EtOAc (50 mL), sequentially washed with a saturated NaHCO₃ solution (50 mL) and a saturated NaCl solution (50 mL), dried (Na₂SO₄), and concentrated under reduced pressure. The residue was purified by column chromatography (gradient from 30% EtOAc/70% hexane to 100% EtOAc) to give the desired product (0.38 g 97%): TLC (50% EtOAc/50% hexane) R_{f} 0.13; ¹H-NMR (CDCl₃) δ 1.26 (s, 9H), 6.65 (d, J=1.48 Hz, 1H), 6.76 (dd, J=1.47, 4.24 Hz, 2H), 6.86 (d, J=1.47 Hz, 1H), 6.91 (d, J=8.82 Hz, 2H), 7.31 (d, J=8.83 Hz, 2H), 8.39 (br s, 2H), 8.41 (d, J=1.47 Hz, 2H); ¹³C-NMR (CDCl₃) δ 32.1 (3C), 34.4, 106.2, 112.0 (2C), 116.6, 121.3 (2C), 121.5 (2C), 134.9, 136.1, 149.0, 151.0 (2C), 154.0, 156.9, 165.2; FAB-MS m/z (rel abundance) 368 ((M+H)⁺, 100%).

CS. General Method for the Reaction of a Substituted Aniline with Triphosgene Followed by Reaction with a Second Substituted Amine

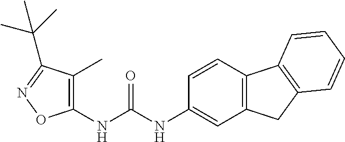

N-(3-tert-Butyl-4-methyl-5-isoxazolyl)-N′-(2-fluorenyl)urea: To a solution of triphosgene (55 mg, 0.185 mmol, 0.37 eq) in 1,2-dichloroethane (1.0 mL) was added a solution of 5-amino-4-methyl-3-tert-butylisoxazole (77.1 mg, 0.50 mmol, 1.0 eq) and diisopropylethylamine (0.104 mL, 0.60 mmol, 1.2 eq) in 1,2-dichloroethane (1.0 mL). The reaction mixture was stirred at 70° C. for 2 h, cooled to room temp., and treated with a solution of 2-aminofluorene (30.6 mg, 0.50 mmol, 1.0 eq) and diisopropylethylamine (0.087 mL, 1.0 eq) in 1,2-dichloroethane (1.0 mL). The reaction mixture was stirred at 40° C. for 3 h and then at RT for 17 h to produce a precipitate. The solids were washed with Et₂O and hexanes to give the desired urea as a beige solid (25 mg, 14%): mp 179-181° C.; ¹H-NMR (DMSO-d₆) δ 1.28 (s, 9H), 2.47 (s, 3H), 3.86 (s, 2H), 7.22 (t, J=7.3 Hz, 1H), 7.34 (m, 2H), 7.51 (d, J=7.3 Hz, 1H), 7.76 (m, 3H), 8.89 (s, 1H), 9.03 (s, 1H); HPLC ES-MS m/z 362 ((M+H)⁺).

### C6. General Method for Urea Formation by Curtius Rearrangement and Carbamate Trapping

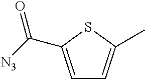

Step 1. 5-Methyl-2-(azidocarbonyl)thiophene: To a solution of 5-Methyl-2-thiophenecarboxylic acid (1.06 g, 7.5 mmol) and Et₃N (1.25 mL, 9.0 mmol) in acetone (50 mL) at −10° C. was slowly added ethyl chloroformate (1.07 mL, 11.2 mmol) to keep the internal temperature below 5° C. A solution of sodium azide (0.83 g, 12.7 mmol) in water (6 mL) was added and the reaction mixture was stirred for 2 h at 0° C. The resulting mixture was diluted with CH₂Cl₂ (10 mL) and washed with a saturated NaCl solution (10 mL). The aqueous layer was back-extracted with CH₂Cl₂ (10 mL), and the combined organic layers were dried (MgSO₄) and concentrated in vacuo. The residue was purified by column chromatography (10% EtOAc/90% hexanes) to give the azidoester (0.94 g, 75%). Azidoester (100 mg, 0.6 mmol) in anhydrous toluene (10 mL) was heated to reflux for 1 h then cooled to rt. This solution was used as a stock solution for subsequent reactions.

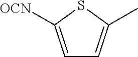

Step 2. 5-Methyl-2-thiophene Isocyanate: 5-Methyl-2-(azidocarbonyl)thiophene (0.100 g, 0.598 mmol) in anh toluene (10 mL) was heated at the reflux temp. for 1 h then cooled to room temp. This solution was used as a stock solution for subsequent reactions.

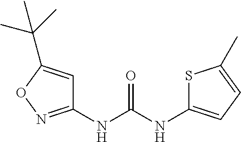

Step 3. N-(5-tert-Butyl-3-isoxazolyl)-N′-(5-methyl-2-thienyl)urea: To a solution of 5-methyl-2-thiophene isocyanate (0.598 mmol) in toluene (10 mL) at room temp. was added 3-amino-5-tert-butylisoxazole (0.092 g, 0.658 mmol) and the resulting mixture was stirred overnight. The reaction mixture was diluted with EtOAc (50 mL) and sequentially washed with a 1 N HCl solution (2×25 mL) and a saturated NaCl solution (25 mL), dried (MgSO₄), and concentrated under reduced pressure. The residue was purified by MPLC (20% EtOAc/80% hexane) to give the desired urea (0.156 g, 93%): mp 200-201° C.; TLC (20% EtOAc/80% hexane) R_{f} 0.20; EI-MS m/z 368 (M⁺).

### C7. General Methods for Urea Formation by Curtius Rearrangement and Isocyanate Trapping

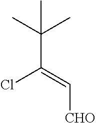

Step 1. 3-Chloro-4,4-dimethylpent-2-enal: POCl₃ (67.2 mL, 0.72 mol) was added to cooled (0° C.) DMF (60.6 mL, 0.78 mol) at rate to keep the internal temperature below 20° C. The viscous slurry was heated until solids melted (approximately 40° C.), then pinacolone (37.5 mL, 0.30 mol) was added in one portion. The reaction mixture was then to 55° C. for 2 h and to 75° C. for an additional 2 h. The resulting mixture was allowed to cool to room temp., then was treated with THF (200 mL) and water (200 mL), stirred vigorously for 3 h, and extracted with EtOAc (500 mL). The organic layer was washed with a saturated NaCl solution (200 mL), dried (Na₂SO₄) and concentrated under reduced pressure. The residue was filtered through a pad of silica (CH₂Cl₂) to give the desired aldehyde as an orange oil (15.5 g, 35%): TLC (5% EtOAc/95% hexane) R_{f} 0.54; NMR (CDCl₃) d 1.26 (s, 9H), 6.15 (d, Hz, 1H), 10.05 (d, J=6.6 Hz, 1H).

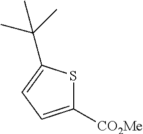

Step 2. Methyl 5-tert-butyl-2-thiophenecarboxylate: To a solution of 3-chloro-4,4-dimethylpent-2-enal (1.93 g, 13.2 mmol) in anh. DMF (60 mL) was added a solution of Na₂S (1.23 g, 15.8 mmol) in water (10 mL). The resulting mixture was stirred at room temp. for 15 min to generate a white precipitate, then the slurry was treated with methyl bromoacetate (2.42 g, 15.8 mmol) to slowly dissolve the solids. The reaction mixture was stirred at room temp. for 1.5 h, then treated with a 1 N HCl solution (200 mL) and stirred for 1 h. The resulting solution was extracted with EtOAc (300 mL). The organic phase was sequentially washed with a 1 N HCl solution (200 mL), water (2×200 mL) and a saturated NaCl solution (200 mL), dried (Na₂SO₄) and concentrated under reduced pressure. The residue was purified using column chromatography (5% EtOAc/95% hexane) to afford the desired product (0.95 g, 36%): TLC (20% EtOAc/80% hexane) R_{f} 0.79; ¹H NMR (CDCl₃) δ 1.39 (s, 9H), 3.85 (s, 3H), 6.84 (d, J=3.7 Hz, 1H), 7.62 (d, j=4.1 Hz, 1H); GC-MS m/z (rel abundance) 198 (M⁺, 25%).

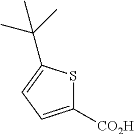

Step 3. 5-tert-Butyl-2-thiophenecarboxylic acid: Methyl 5-tert-butyl-2-thiophenecarboxylate (0.10 g, 0.51 mmol) was added to a KOH solution (0.33 M in 90% MeOH/10% water, 2.4 mL, 0.80 mmol) and the resulting mixture was heated at the reflux temperature for 3 h. EtOAc (5 mL) was added to the reaction mixture, then the pH was adjusted to approximately 3 using a 1 N HCl solution. The resulting organic phase was washed with water (5 mL), dried (Na₂SO₄), and concentrated under reduced pressure (0.4 mmHg) to give the desired carboxylic acid as a yellow solid (0.067 g, 73%): TLC (20% EtOAc/79.5% hexane/0.5% AcOH) R_{f} 0.29; ¹H NMR (CDCl₃) δ 1.41 (s, 9H), 6.89 (d, J=3.7 Hz, 1H), 7.73 (d, J=3.7 Hz, 1H), 12.30 (br s, 1H); ¹³C NMR (CDCl₃) δ 32.1 (3C), 35.2, 122.9, 129.2, 135.1, 167.5, 168.2.

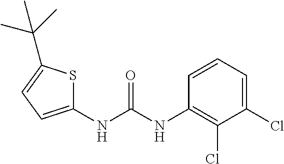

Step 4. N-(5-tort-Butyl-2-thienyl)-N′-(2,3-dichlorophenyl)urea: A mixture of 5-tert-butyl-2-thiophenecarboxylic acid (0.066 g, 0.036 mmol), DTPA (0.109 g, 0.39 mmol) and Et₃N (0.040 g, 0.39 mmol) in toluene (4 mL) was heated to 80° C. for 2 h, 2,3-dichloroaniline (0.116 g, 0.72 mmol) was added, and the reaction mixture was heated to 80° C. for an additional 2 h. The resulting mixture was allowed to cool to room temp. and treated with EtOAc (50 mL). The organic layer was washed with a 1 N HCl solution (3×50 mL), a saturated NaHCO₃ solution (50 mL), and a saturated NaCl solution (50 mL), dried (Na₂SO₄), and concentrated under reduced pressure. The residue was purified by column chromatography (5% EtOAc/95% hexane) to afford the desired urea as a purple solid (0.030 g, 24%): TLC (10% EtOAc/90% hexane) R_{f} 0.28; ¹H NMR, (CDCl₃) δ 1.34 (s, 9H), 6.59 (br s, 2H), 7.10-7.13 (m, 2H), 7.66 (br s, 1H), 8.13 (dd, J=2.9, 7.8 Hz, 1H); ¹³C NMR (CDCl₃) δ 32.2 (3C), 34.6, 117.4, 119.0⁷, 119.1⁵, 119.2, 121.5, 124.4, 127.6, 132.6, 135.2, 136.6, 153.4; HPLC ES-MS m/z (rel abundance) 343 ((M+H)⁺, 100%), 345 ((M+H+2)⁺, 67%), 347 ((M+H+4)⁺, 14%).

### C8. Combinatorial Method for the Synthesis of Diphenyl Ureas Using Triphosgene

One of the anilines to be coupled was dissolved in dichloroethane (0.10 M). This solution was added to a 8 mL vial (0.5 mL) containing dichloroethane (1 mL). To this was added a triphosgene solution (0.12 M in dichloroethane, 0.2 mL, 0.4 equiv.), followed by diisopropylethylamine (0.35 M in dichloroethane, 0.2 mL, 1.2 equiv.). The vial was capped and heat at 80° C. for 5 h, then allowed to cool to room temp for approximately 10 h. The second aniline was added (0.10 M in dichloroethane, 0.5 mL, 1.0 equiv.), followed by diisopropylethylamine (0.35 M in dichloroethane, 0.2 mL, 1.2 equiv.). The resulting mixture was heated at 80° C. for 4 h, cooled to room temperature and treated with MeOH (0.5 mL). The resulting mixture was concentrated under reduced pressure and the products were purified by reverse phase HPLC.

D. Misc. Methods of Urea Synthesis

### D1. Electrophylic Halogenation

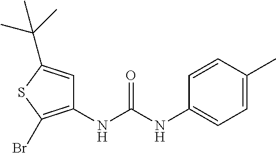

N-(2-Bromo-5-tert-butyl-3-thienyl)-N′-(4-methylphenyl)urea: To a slurry of N-(5-tert-butyl-3-thienyl)-N′-(4-methylphenyl)urea (0.50 g, 1.7 mmol) in CHCl₃ (20 mL) at room temp was slowly added a solution of Br, (0.09 mL, 1.7 mmol) in CHCl₃ (10 mL) via addition funnel causing the reaction mixture to become homogeneous. Stirring was continued 20 min after which TLC analysis indicated complete reaction. The reaction was concentrated under reduced pressure, and the residue triturated (2×Et₂O/hexane) to give the brominated product as a tan powder (0.43 g, 76%): nip 161-163° C.; TLC (20% EtOAc/80% hexane) δ 0.71; ¹H NMR (DMSO-d₆) δ 1.29 (s, 9H), 2.22 (s, 3H), 7.07 (d, J=8.46 Hz, 2H), 7.31 (d, J=8.46 Hz, 2H), 7.38 (s, 1H), 8.19 (s, 1H), 9.02 (s, 1H); ¹³C NMR (DMSO-d₆) δ 20.3, 31.6 (3C), 34.7, 89.6, 117.5, 118.1 (2C), 129.2 (2C), 130.8, 136.0, 136.9, 151.8, 155.2; FAB-MS m/z (rel abundance) 367 ((M+H)⁺, 98%), 369 (M+2+H)⁺, 100%).

D2. Synthesis of co-Alkoxy Ureas

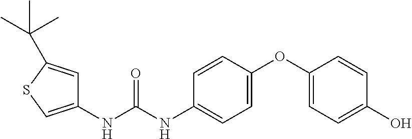

Step 1. N-(5-tert-Butyl-3-thienyl)-N′-(4-(4-hydroxyphenyl)oxyphenyl)urea: A solution of N-(5-tert-butyl-3-thienyl)-N′-(4-(4-methoxyphenyl)oxyphenyl)urea (1.2 g, 3 mmol) in CH₂Cl₂ (50 mL) was cooled to −78° C. and treated with BBr₃ (1.0 M in CH₂Cl₂, 4.5 mL, 4.5 mmol, 1.5 equiv) dropwise via syringe. The resulting bright yellow mixture was warmed slowly to room temp and stirred overnight. The resulting mixture was concentrated under reduced pressure. The residue was dissolved in EtOAc (50 mL), then washed with a saturated NaHCO₃ solution (50 mL) and a saturated NaCl solution (50 mL), dried (Na₂SO₄), and concentrated under reduced pressure. The residue was purified via flash chromatography (gradient from 10% EtOAc/90% hexane to 25% EtOAc/75% hexane) to give the desired phenol as a tan foam (1.1 g, 92%): TLC (20% EtOAc/80% hexane) R_{f} 0.23; ¹H NMR (DMSO-d₆) 1.30 (s, 9H), 6.72-6.84 (m, 7H), 6.97 (d, J=1.47 Hz, 1H), 7.37 (dm, J=9.19 Hz, 2H), 8.49 (s, 1H), 8.69 (s, 1H), 9.25 (s, 1H); FAB-MS m/z (Tel abundance) 383 ((M+H)⁺, 33%).

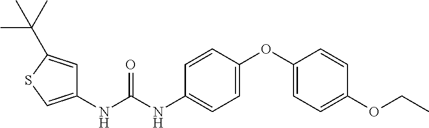

Step 2. N-(5-tert-Butyl-3-thienyl)-N′-(4-(4-ethoxyphenyl)oxyphenyl)urea: To a mixture of N-(5-tert-butyl-3-thienyl)-N′-(4-(4-hydroxyphenyl)oxyphenyl)urea (0.20 g, 0.5 mmol) and Cs₂CO₃ (0.18 g, 0.55 mmol, 1.1 equiv) in reagent grade acetone (10 mL) was added ethyl iodide (0.08 mL, 1.0 mmol, 2 equiv) via syringe, and the resulting slurry was heated at the reflux temp, for 17 h. The reaction was cooled, filtered, and the solids were washed with EtOAc. The combined organics were concentrated under reduced pressure, and the residue was purified via preparative HPLC (60% CH₃CN/40% H₂O/0.05% TFA) to give the desired urea as a colorless powder (0.16 g, 73%): mp 155-156° C.; TLC (20% EtOAC/80% hexane) R_{f} 0.40; ¹H-NMR (DMSO-d₆) δ 1.30 (s, 9H), 1.30 (t, J=6.99 Hz, 3H), 3.97 (q, J=6.99 Hz, 2H), 6.80 (d, J=1.47 Hz, 1H), 6.86 (dm, J=8.82 Hz, 2H), 6.90 (s, 4H), 6.98 (d, J=1.47, 1H), 7.40 (dm, J=8.83 Hz, 2H), 8.54 (s, 1H), 8.73 (s, 1H); ¹³C-NMR (DMSO-d₆) δ 14.7, 32.0 (3C), 33.9, 63.3, 102.5, 115.5 (2C), 116.3, 118.4 (2C), 119.7 (2C), 119.8 (2C), 135.0, 136.3, 150.4, 152.1, 152.4, 154.4, 154.7; FAB-MS m/z (rel abundance) 411 ((M+H)⁺, 15%).

### D3. Synthesis of O-Carbamoyl Ureas

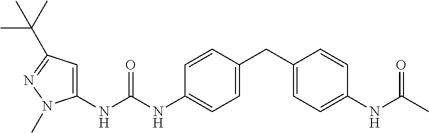

N-(3-tert-Butyl-1-methyl-5-pyrazolyl)-N′-(4-(4-acetaminophenyl)methylphenyl)urea: To a solution of N-(3-tert-butyl-1-methyl-5-pyrazolyl)-N′-(4-(4-aminophenyl)methylphenyl)urea (0.300 g, 0.795 mmol) in CH₂Cl_{z} (15 mL) at 0° C. was added acetyl chloride (0.057 mL, 0.795 mmol), followed by anhydrous Et₃N (0.111 mL, 0.795 mmol). The solution was allowed to warm to room temp over 4 h, then was diluted with EtOAc (200 mL). The organic layer was sequentially washed with a 1M HCl solution (125 mL) then water (100 mL), dried (MgSO₄), and concentrated under reduced pressure. The resulting residue was purified by filtration through a pad of silica (EtOAc) to give the desired product as a white solid (0.160 g, 48%): TLC (EtOAc) R_{f} 0.33; ¹H-NMR (DMSO-d₆) δ 1.17 (s, 9H), 1.98 (s, 3H), 3.55 (s, 3H), 3.78 (s, 2H), 6.00 (s, 1H), 7.07 (d, J=8.5 Hz, 2H), 7.09 (d, J=8.5 Hz, 2H), 7.32 (d, J=8.5 Hz, 2H), 7.44 (d, J=8.5 Hz, 2H), 8.38 (s, 1H), 8.75 (s, 1H), 9.82 (s, 1H); FAB-MS m/z 420 ((M+H)⁺).

### D4. General Method for the Conversion of Ester-Containing Ureas Into Alcohol-Containing Ureas

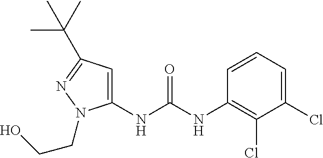

N—(N¹-(2-Hydroxyethyl)-3-tert-butyl-5-pyrazolyl)-N′-(2,3-dichlorophenyl)urea: A solution of N—(N¹-(2-(2,3-dichlorophenylamino)carbonyloxyethyl)-3-tert-butyl-5-pyrazolyl)-N′-(2,3-dichlorophenyl)urea (prepared as described in Method A3; 0.4 g, 0.72 mmoles) and NaOH (0.8 mL, 5N in water, 4.0 mmoles) in EtOH (7 mL) was heated at ˜65° C. for 3 h at which time TLC indicated complete reaction. The reaction mixture was diluted with EtOAc (25 mL) and acidified with a 2N HCl solution (3 mL). The resulting organic phase was washed with a saturated NaCl solution (25 mL), dried (MgSO₄) and concentrated under reduced pressure. The residue was crystallized (Et₂O) to afford the desired product as a white solid (0.17 g, 64%); TLC (60% EtOAc/40% hexane) R_{f} 0.16; ¹H-NMR (DMSO-d₆) δ 1.23 (s, 9H), 3.70 (t, J=5.7 Hz, 2H), 4.10 (t, J=5.7 Hz, 2H), 6.23 (s, 1H), 7.29-7.32 (m, 2H), 8.06-8.09 (m, 1H), 9.00 (hr s, 1H), 9.70 (br s, 1H); FAB-MS m/z (rel abundance) 371 ((M+H)⁺, 100%),

D5a. General Method for the Conversion of Ester-Containing Ureas into Amide-Containing Ureas

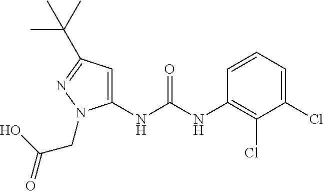

Step 1. N—(N¹-(Carboxymethyl)-3-tert-butyl-5-pyrazolyl)-N′-(2,3-dichlorophenyl)urea: A solution of N—(N′-(ethoxycaxbonylmethyl)-3-tert-butyl-5-pyrazolyl)-N′-(2,3-dichlorophenyl)urea (prepared as described in Method A3, 0.46 g, 1.11 moles) and NaOH (1.2 mL, 5N in water, 6.0 mmoles) in EtOH (7 mL) was stirred at room temp. for 2 h at which time TLC indicated complete reaction. The reaction mixture was diluted with EtOAc (25 mL) and acidified with a 2N HCl solution (4 mL). The resulting organic phase was washed with a saturated NaCl solution (25 mL), dried (MgSO₄) and concentrated under reduced pressure. The residue was crystallized (Et₂O/hexane) to afford the desired product as a white solid (0.38 g, 89%): TLC (10% MeOH/90% CH₂Cl₂) R_{f} 0.04; ¹H-NMR (DMSO-d₆) δ 1.21 (s, 9H), 4.81 (s, 2H), 6.19 (s, 1H), 7.28-7.35 (m, 2H), 8.09-8.12 (m, 1H), 8.76 (br s, 1H), 9.52 (br s, 1H); FAB-MS m/z (rel abundance) 385 ((M+H)⁺, 100%).

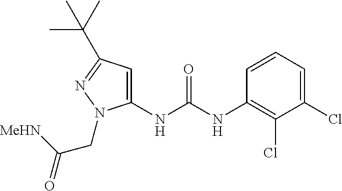

Step 2. N—(N¹-(Methylcarbamoyl)methyl)-3-tert-butyl-5-pyrazolyl)-N′-(2,3-dichlorophenyl)urea: A solution of N—(N¹-(carboxymethyl)-3-tert-butyl-5-pyrazolyl)-N′-(2,3-dichlorophenyl)urea (100 mg, 0.26 mmole) and N,N′-carbonyldiimidazole (45 mg, 0.28 mmole) in CH₂Cl₂ (10 mL) was stirred at room temp. 4 h at which time TLC indicated formation of the corresponding anhydride (TLC (50% acetone/50% CH₂Cl₂) R_{f} 0.81). Dry methylamine hydrochloride (28 mg, 0.41 mmole) was then added followed by of diisopropylethylamine (0.07 mL, 0.40 mmole). The reaction mixture was stirred at room temp. overnight, then diluted with CH₂Cl₂, washed with water (30 mL), a saturated NaCl solution (30 mL), dried (MgSO₄) and concentrated under reduced pressure. The residue was purified by column chromatography (gradient from 10% acetone/90% CH₂Cl₂ to 40% acetone/60% CH₂Cl₂) and the residue was crystallized (Et₂O/hexane) to afford the desired product (47 mg, 46%): TLC (60% acetone/40% CH₂Cl₂) R_{f} 0.59; ¹H-NMR (DMSO-d₆) δ 1.20 (s, 9H), 2.63 (d, J=4.5 Hz, 3H), 4.59 (s, 2H), 6.15 (s, 1H), 7.28-7.34 (m, 2H), 8.02-8.12 (m, 2H), 8.79 (br s, 1H), 9.20 (br s, 1H); FAB-MS m/z (rel abundance) 398 ((M+H)⁺, 30%).

D5b. General Method for the Conversion of Ester-Containing Ureas into Amide-Containing Ureas

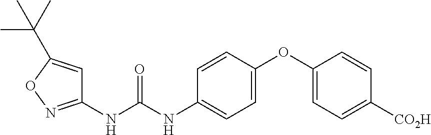

Step 1. N-(5-tert-Butyl-3-isoxazolyl)-N′-(4-(4-carboxyphenyl)oxyphenyl)urea: To a solution of N-(5-tert-butyl-3-isoxazolyl)-N′-(4-(4-ethoxyoxycarbonylphenyl)-oxyphenyl)urea (0.524 g, 1.24 mmol) in a mixture of EtOH (4 mL) and TIT (4 mL) was added a 1M NaOH solution (2 mL) and the resulting solution was allowed to stir overnight at room temp. The resulting mixture was diluted with water (20 mL) and treated with a 3M HCl solution (20 mL) to form a white precipitate. The solids were washed with water (50 mL) and hexane (50 mL), and then dried (approximately 0.4 mmHg) to afford the desired product (0.368 g, 75%). This material was carried to the next step without further purification.

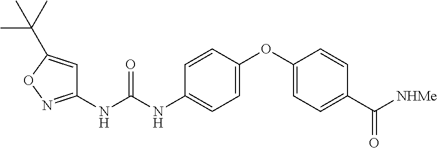

Step 2. N-(5-tert-Butyl-3-isoxazolyl)-N′-(4-(4-(N-methylcarbamoyl)-phenyl)oxyphenyl)urea: A solution of N-(5-tert-butyl-3-isoxazolyl)-N′-(4-(4-carboxyphenyl)oxyphenyl)urea (0.100 g, 0.25 mmol), methylamine (2.0 M in THF; 0.140 mL, 0.278 mmol), 1-ethyl-3-(3-dimethylaminopropyl)carbodiimide hydrochloride (76 mg, 0.39 mmol), and N-methylmorpholine (0.030 mL, 0.27 mmol) in a mixture of THF (3 mL) and DMF (3 mL) was allowed to stir overnight at room temp. then was poured into a 1M citric acid solution (20 mL) and extracted with EtOAc (3×15 mL). The combined extracts were sequentially washed with water (3×10 mL) and a saturated NaCl solution (2×10 mL), dried (Na₂SO₄), filtered, and concentrated in vacuo. The resulting crude oil was purified by flash chromatography (60% EtOAc/40% hexane) to afford the desired product as a white solid (42 mg, 40%): ET-MS m/z 409 ((M+H)⁺).

D6. General Method for the Conversion of ω-Amine-Containing Ureas into Amide-Containing Ureas

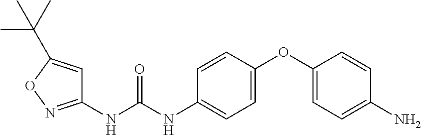

N-(5-tert-Butyl-3-isoxazolyl)-N′-(4-(4-aminophenyl)oxyphenyl)urea: To a solution of N-(5-tert-butyl-3-isoxazolyl)-N′-(4-(4-tert-butoxycarbonylaminophenyl)oxy-phenyl)-urea (prepared in a manner analogous to Methods B6 then C2b; 0.050 g, 0.11 mmol) in anh 1,4-dioxane (3 mL) was added a cone HCl solution (1 mL) in one portion and the mixture was allowed to stir overnight at room temp. The mixture was then poured into water (10 mL) and EtOAc (10 mL) and made basic using a 1M NaOH solution (5 mL), The aqueous layer was extracted with EtOAc (3×10 mL). The combined organic layers were sequentially washed with water (3×100 mL) and a saturated NaCl solution (2×100 mL), dried (Na₂SO₄), and concentrated in vacuo to afford the desired product as a white solid (26 mg, 66%). EI-MS m/z 367 ((M+H)⁺).

### D7. General Method for the Oxidation of Pyridine-Containing Ureas

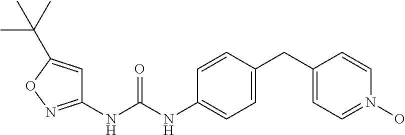

N-(5-tert-Butyl-3-isoxazolyl)-N′-(4-(N-oxo-4-pyridinyl)methylphenyl)urea: To a solution of N-(5-tert-butyl-3-isoxazolyl)-N′-(4-(4-pyridinyl)methylphenyl)urea (0.100 g, 0.29 mmol) in CHCl₃ (10 mL) was added m-CPBA (70% pure, 0.155 g, 0.63 mmol) and the resulting solution was stirred at room temp for 16 h. The reaction mixture was then treated with a saturated K₂CO₃ solution (10 mL). After 5 min, the solution was diluted with CHCl₃ (50 mL). The organic layer was washed successively with a saturated aqueous NaHSO₃ solution (25 mL), a saturated NaHCO₃ solution (25 mL) and a saturated NaCl solution (25 mL), dried (MgSO₄), and concentrated in vacuo. The residual solid was purified by MPLC (15% MeOH/85% EtOAc) to give the N-oxide (0.082 g, 79%).

### D8. General Method for the Acylation of a Hydroxy-Containing Urea

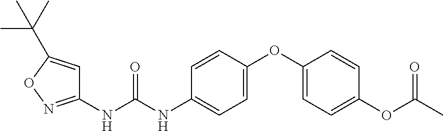

N-(5-tert-Butyl-3-isoxazolyl)-N′-(4-(4-acetoxyphenyloxy)phenyl)urea: To a solution of N-(5-tert-butyl-3-isoxazolyl)-N′-(4-(4-hydroxyphenyloxy)phenyl)urea (0.100 g, 0.272 mmol), N,N-dimethylaminopyridine (0.003 g, 0.027 mmol) and Et₃N (0.075 mL, 0.544 mmol) in anh THF (5 mL) was added acetic anhydride (0.028 mL, 0.299 mmol), and the resulting mixture was stirred at room temp. for 5 h. The resulting mixture was concentrated under reduced pressure and the residue was dissolved in EtOAc (10 mL). The resulting solution was sequentially washed with a 5% citric acid solution (10 mL), a saturated NaHCO₃ solution (10 mL) and a saturated NaCl solution (10 mL), dried (Na₂SO₄), and concentrated under reduced pressure to give an oil which slowly solidified to a glass (0.104 g, 93%) on standing under reduced pressure (approximately 0.4 mmHg): TLC (40% EtOAc/60% hexane) R_{f} 0.55; FAB-MS m/z 410 ((M+H)⁺).

### D9. Synthesis of ω-Alkoxypyridines

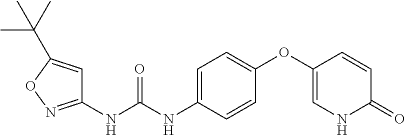

Step 1. N-(5-tert-Butyl-3-isoxazolyl)-N′-(4-(2(1H)-pyridinon-5-yl)oxyphenyl)-urea: A solution of N-(5-tert-butyl-3-isoxazolyl)-N′-(4-(5-(2-methoxy)pyridyl)-oxyaniline (prepared in a manner analogous to that described in Methods B3k and C3b; 1.2 g, 3.14 mmol) and trimethylsilyl iodide (0.89 mL, 6.28 mmol) in CH₂Cl₂ (30 mL) was allowed to stir overnight at room temp., then was to 40° C. for 2 h. The resulting mixture was concentrated under reduced pressure and the residue was purified by column chromatography (gradient from 80% EtOAc/20% hexans to 15% MeOH/85% EtOAc) to give the desired product (0.87 g, 75%): mp 175-180° C.; TLC (80% EtOAc/20% hexane) R_{f} 0.05; FAB-MS m/z 369 ((M+H)⁺, 100%).

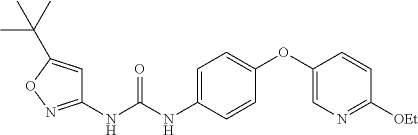

Step 2. N-(5-tert-Butyl-3-isoxazolyl)-N′-(4-(5-(2-Ethoxy)pyridyl)oxyphenyl)urea: A slurry of N-(5-tert-butyl-3-isoxazolyl)-N′-(4-(2 (1H)-pyridinon-5-yl)oxyphenyl)urea (0.1 g, 0.27 mmol) and Ag₂CO₃ (0.05 g, 0.18 mmol) in benzene (3 mL) was stirred at room temp. for 10 min. Iodoethane (0.023 mL, 0.285 mmol) was added and the resulting mixture was heated at the reflux temp. in dark overnight. The reaction mixture was allowed to cool to room temp., and was filtered through a plug of Celite® then concentrated under reduced pressure. The residue was purified by column chromatography (gradient from 25% EtOAc/75% hexane to 40% EtOAc/60% hexane) to afford the desired product (0.041 g, 38%): mp 146° C.; TLC (40% EtOAc/60% hexane) R_{f} 0.49; FAB-MS m/z 397 ((M+H)⁺, 100%).

### D10. Reduction of an Aldehyde- or Ketone-Containing Urea to a Hydroxide-Containing Urea

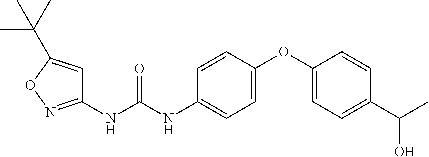

N-(5-tert-Butyl-3-isoxazolyl)-N′44-(4-(1-hydroxyethyl)phenyl)oxyphenyl)urea: To a solution of N-(5-tert-butyl-3-isoxazolyl)-N′-(4-(4-(1-acetylphenyl)oxyphenyl)urea (prepared in a manner analogous to that described in Methods B1 and C2b; 0.060 g, 0.15 mmol) in MeOH (10 mL) was added NaBH₄ (0.008 g, 0.21 mmol) in one portion. The mixture was allowed to stir for 2 h at room temp., then was concentrated in vacuo. Water (20 mL) and a 3M HCl solution (2 nit) were added and the resulting mixture was extracted with EtOAc (3×20 mL). The combined organic layers were washed with water (3×10 mL) and a saturated NaCl solution (2×10 mL), dried (MgSO₄), and concentrated in vacuo. The resulting white solid was purified by trituration (Et₂O/hexane) to afford the desired product (0.021 g, 32%): mp 80-85° C. ¹H NMR (DMSO-d₆) δ 1.26 (s, 9H), 2.50 (s, 3H), 4.67 (m, 1H), 5.10 (br s, 1H), 6.45 (s, 1H), 6.90 (m, 4H), 7.29 (d, J=9.0 Hz, 2H), 7.42 (d, J=9.0 Hz, 2H), 8.76 (s, 1H), 9.44 (s, 1H); HPLC ES-MS m/z 396 ((M+H)⁺).

### D11. Synthesis of Nitrogen-Substituted Ureas by Curtius Rearrangement of Carboxy-Substituted Ureas

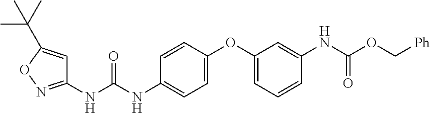

N-(5-tert-Butyl-3-isoxazolyl)-N′-(4-(3-(benzyloxycarbonylamino)phenyl)-oxyphenyl)urea: To a solution of the N-(5-tert-butyl-3-isoxazolyl)-N′-(4-(3-carboxyphenyl)oxyphenyl)urea (prepared in a manner analogous to that described in Methods B3a, Step 2 and C2b; 1.0 g, 2.5 mmol) in anh toluene (20 mL) was added Et₃N (0.395 mL, 2.8 mmol) and DPPA (0.610 mL, 2.8 mmol). The mixture was heated at 80° C. with stirring for 1.5 h then allowed to cool to room temp. Benzyl alcohol (0.370 mL, 3.5 mmol) was added and the mixture was heated at 80° C. with stirring for 3 h then allowed to cool to room temp. The resulting mixture was poured into a 10% HCl solution (50 mL) and the resulting solution extracted with EtOAc (3×50 mL). The combined organic layers were washed with water (3×50 mL) and a saturated NaCl (2×50 mL), dried (Na₂SO₄), and concentrated in vacuo. The crude oil was purified by column chromatography (30% EtOAc/70% hexane) to afford the desired product as a white solid (0.7 g, 60%): mp 73-75° C.; ¹H NMR (DMSO-d₆) δ 1.26 (s, 9H), 5.10 (s, 2H), 6.46 (s, 1H), 6.55 (d, J=7.0 Hz, 1H), 6.94 (d, J=7.0 Hz, 2H), 7.70 (m, 7H), 8.78 (s, 1H), 9.46 (s, 1H), 9.81 (s, 1H); HPLC ES-MS m/z 501 ((M+H)⁺).

The following compounds have been synthesized according to the General Methods listed above:

TABLE 1
5-Substituted-3-isoxazolyl Ureas

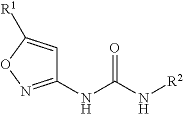

| | | | | | | Mass | |
|---|---|---|---|---|---|---|---|
| | | | mp | TLC | Solvent | Spec. | Synth. |
| Entry | R¹ | R² | (° C.) | R_{f} | System | [Source] | Method |
| 1 | t-Bu | 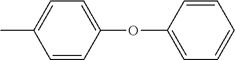 | 148- 149 | | | 352 (M + H)+ [FAB] | C1c |
| 2 | t-Bu | 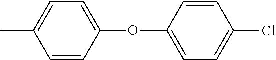 | 176- 177 | 0.16 | 5% MeOH/ 95% CH2Cl2 | 386 (M + H)+ [FAB] | C2b |
| 3 | t-Bu | 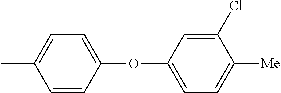 | | 0.50 | 30% EtOAc/ 70% hexane | 400 (M + H)+ [HPLC ES-MS] | C2b |
| 4 | t-Bu | 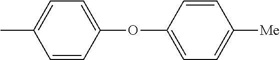 | 156- 157 | 0.50 | 30% EtOAc/ 70% hexane | 366 (M + H)+ [HPLC ES-MS] | C2b |
| 5 | t-Bu | 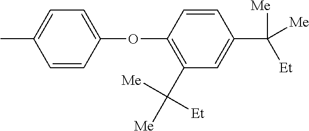 | | 0.80 | 40% EtOAc/ 60% hexane | 492 (M + H)+ [HPLC ES-MS] | C2b |
| 6 | t-Bu | 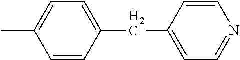 | 190- 191 | 0.15 | 30% EtOAc/ 70% hexane | 350 (M+) [EI] | C2b |
| 7 | t-Bu | 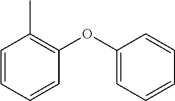 | | 0.55 | 20% EtOAc/ 80% hexane | 352 (M + H)+ [FAB] | C2b |
| 8 | t-Bu | 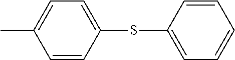 | | 0.25 | 20% EtOAc/ 80% hexane | 367 (M+) [EI] | C2b |
| 9 | t-Bu | 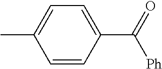 | | 0.15 | 20% EtOAc/ 80% hexane | 363 (M+) [EI] | C2b |
| 10 | t-Bu | 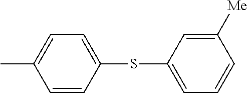 | | 0.30 | 20% EtOAc/ 80% hexane | 381 (M+) [EI] | C2b |
| 11 | t-Bu | 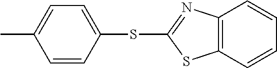 | | 0.25 | 30% EtOAc/ 70% hexane | 425 (M + H)+ [HPLC ES-MS] | B3b, C2b |
| 12 | t-Bu | 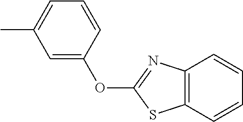 | 175- 177 | 0.25 | 30% EtOAc/ 70% hexane | 409 (M + H)+ [HPLC ES-MS] | B3a, Step 1, B3b Step 2, C2b |
| 13 | t-Bu | 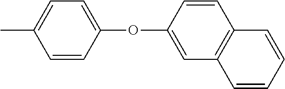 | | 0.35 | 30% EtOAc/ 70% hexane | 402 (M + H)+ [HPLC ES-MS] | B3b, C2b |
| 14 | t-Bu | 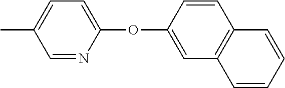 | | 0.20 | 30% EtOAc/ 70% hexane | 403 (M + H)+ [HPLC ES-MS] | B3b, C2b |
| 15 | t-Bu | 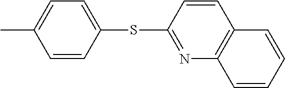 | | 0.25 | 30% EtOAc/ 70% hexane | 419 (M + H)+ [HPLC ES-MS] | B3b, C2b |
| 16 | t-Bu | 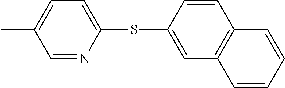 | | 0.20 | 30% EtOAc/ 70% hexane | 419 (M + H)+ [HPLC ES-MS] | B3b, C2b |
| 17 | t-Bu | 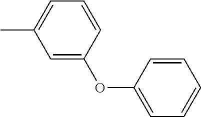 | | 0.40 | 30% EtOAc/ 70% hexane | 352 (M + H)+ [HPLC ES-MS] | C2b |
| 18 | t-Bu | 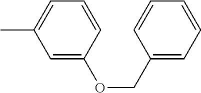 | | 0.40 | 30% EtOAc/ 70% hexane | 365 (M+) [EI] | C2b |
| 19 | t-Bu | 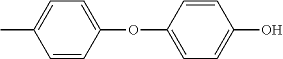 | | 0.15 | 30% EtOAc/ 70% hexane | 367 (M+) [EI] | B3a, C2b, D2 Step 1 |
| 20 | t-Bu | 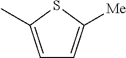 | 200- 201 | 0.20 | 20% EtOAc/ 80% hexane | 280 (M + H)+ [FAB] | C6 |
| 21 | t-Bu | 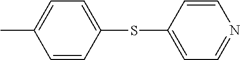 | 178- 179 | | | 368 (M+) [EI] | B4a, C2b |
| 22 | t-Bu | 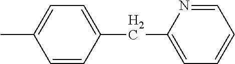 | 164- 165 | 0.25 | 30% EtOAc/ 70% hexane | 351 (M + H)+ [FAB] | B1, C2b |
| 23 | t-Bu | 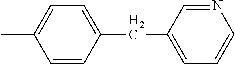 | 170- 172 | 0.15 | 30% EtOAc/ 70% hexane | 351 (M + H)+ [FAB] | B7, B1, C2b |
| 24 | t-Bu | 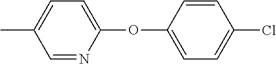 | 179- 182 | 0.20 | 30% EtOAc/ 70% hexane | 387 (M + H)+ [FAB] | C2b |
| 25 | t-Bu | 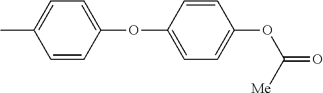 | | 0.55 | 40% EtOAc/ 60% hexane | 410 (M + H)+ [FAB] | B3b, C2b, D2 Step 1, D8 |
| 26 | t-Bu | 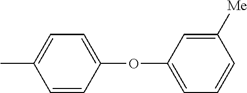 | 176- 182 | 0.55 | 25% EtOAc/ 75% hexane | 366 (M + H)+ [FAB] | B3a, C2b |
| 27 | t-Bu | 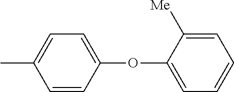 | | 0.40 | 25% EtOAc/ 75% hexane | 366 (M + H)+ [FAB] | B3a, C2b |
| 28 | t-Bu | 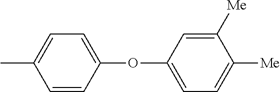 | 150- 158 | 0.45 | 25% EtOAc/ 75% hexane | 380 (M + H)+ [FAB] | B3a, C2b |
| 29 | t-Bu | 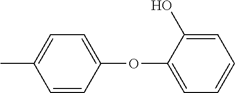 | | 0.30 | 25% EtOAc/ 75% hexane | 368 (M + H)+ [FAB] | C2b |
| 30 | t-Bu | 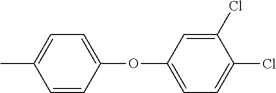 | 118- 122 | 0.50 | 25% EtOAc/ 75% hexane | 420 (M + H)+ [FAB] | B3a Step 1, B3b Step 2, C2b |
| 31 | t-Bu | 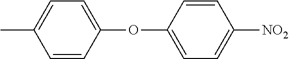 | 195- 197 | 0.30 | 25% EtOAc/ 75% hexane | 397 (M+) [FAB] | C2b |
| 32 | t-Bu | 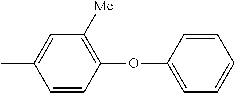 | | 0.80 | 25% EtOAc/ 75% hexane | 366 (M + H)+ [FAB] | B3a, C2b |
| 33 | t-Bu | 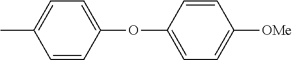 | 155- 156 | 0.55 | 30% EtOAc/ 70% hexane | 382 (M + H)+ [FAB] | B3a, C2b |
| 34 | t-Bu | 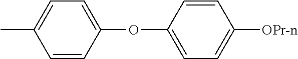 | 137- 141 | 0.62 | 25% EtOAc/ 75% hexane | 410 (M + H)+ [FAB] | B3a, C2b, D2 |
| 35 | t-Bu | 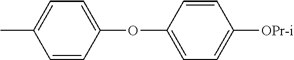 | 164- 166 | 0.60 | 25% EtOAc/ 75% hexane | 410 (M + H)+ [FAB] | B3a, C2b, D2 |
| 36 | t-Bu | 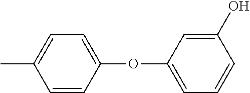 | 78- 80 | 0.15 | 25% EtOAc/ 75% hexane | 368 (M + H)+ [FAB] | C2b |
| 37 | t-Bu | 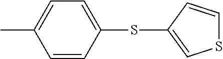 | 167- 169 | | | 374 (M + H)+ [FAB] | B3i, B1, C2b |
| 38 | t-Bu | 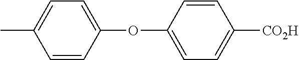 | 200 dec | 0.30 | 5% MeOH/ 0.5% AcOH/ 94.5% CH2Cl2 | 396 (M + H)+ [FAB] | B3a Step 2, C2b |
| 39 | t-Bu | 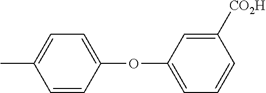 | 234 dec | 0.30 | 5% MeOH/ 0.5% AcOH/ 94.5% CH2Cl2 | 396 (M + H)+ [FAB] | B3a Step 2, C2b |
| 40 | t-Bu | 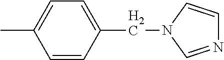 | 203- 206 | 0.35 | 10% MeOH 0.5% AcOH/ 89.5% EtOAc | 340 (M + H)+ [FAB] | B8, B2b, C2b |
| 41 | t-Bu | 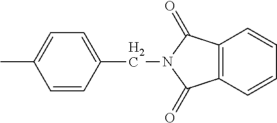 | 177- 180 | | | 419 (M + H)+ [FAB] | B8, B2b, C2b |
| 42 | t-Bu | 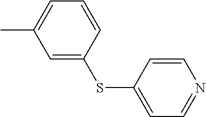 | 158- 159 | 0.25 | 30% EtOAc/ 70% hexane | 369 (M + H)+ [FAB] | B4a, C2b |
| 43 | t-Bu | 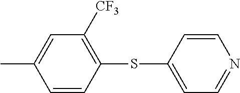 | 180- 181 | 0.15 | 30% EtOAc/ 70% hexane | 437 (M + H)+ [FAB] | B4a, C2b |
| 44 | t-Bu | 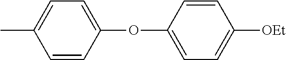 | 140- 142 | 0.25 | 20% EtOAc/ 80% hexane | 396 (M + H)+ [FAB] | B3a, C2b, D2 |
| 45 | t-Bu | 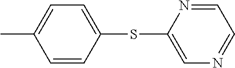 | 68- 71 | 0.30 | 50% EtOAc/ 50% hexane | 370 (M + H)+ [FAB] | B4a, C2b |
| 46 | t-Bu | 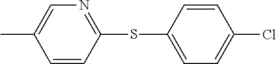 | 183- 186 | 0.30 | 30% EtOAc/ 70% hexane | 403 (M + H)+ [CI] | C2b |
| 47 | t-Bu | 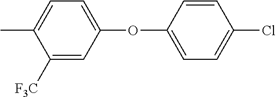 | 98- 101 | 0.25 | 10% EtOAc/ 90% hexane | 454 (M + H)+ [FAB] | C2b |
| 48 | t-Bu | 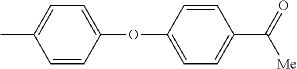 | 163- 166 | 0.25 | 20% EtOAc/ 80% hexane | 394 (M + H)+ [FAB] | B1, C2b |
| 49 | t-Bu | 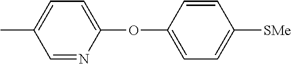 | 144- 147 | 0.25 | 20% EtOAc/ 80% hexane | 399 (M + H)+ [FAB] | C2b |
| 50 | t-Bu | 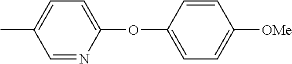 | 155- 157 | 0.25 | 40% EtOAc/ 60% hexane | 383 (M + H)+ [FAB] | C2b |
| 51 | t-Bu | 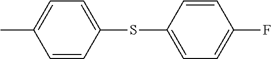 | 162- 164 | 0.35 | 25% EtOAc/ 75% hexane | 386 (M + H)+ [FAB] | C2b |
| 52 | t-Bu | 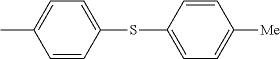 | 149- 150 | 0.15 | 15% EtOAc/ 85% hexane | 382 (M + H)+ [FAB] | C2b |
| 53 | t-Bu | 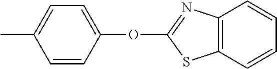 | 77- 80 | 0.30 | 30% EtOAc/ 70% hexane | 408 (M+) [EI] | B3e, C2b |
| 54 | t-Bu | 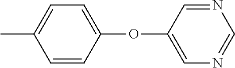 | 162- 164 | 0.17 | 40% EtOAc/ 60% hexane | 354 (M + H)+ [FAB] | B3j, C2b |
| 55 | t-Bu | 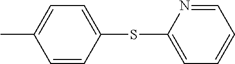 | 73- 76 | 0.20 | 30% EtOAc/ 70% hexane | 368 (M+) [EI] | B2, C2b |
| 56 | t-Bu | 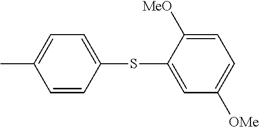 | 73- 75 | 0.15 | 25% EtOAc/ 75% hexane | 428 (M + H)+ [FAB] | B2, C2b |
| 57 | t-Bu | 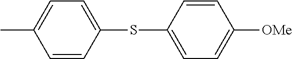 | 143- 145 | 0.25 | 30% EtOAc/ 70% hexane | 398 (M + H)+ [FAB] | B3e, C2b |
| 58 | t-Bu | 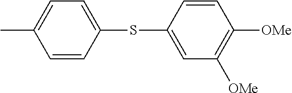 | 148- 151 | 0.25 | 30% EtOAc/ 70% hexane | 428 (M + H)+ [FAB] | B3e, C2b |
| 59 | t-Bu | 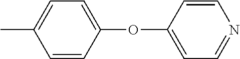 | | 0.30 | 100% EtOAc | 353 (M + H)+ [FAB] | B4b, C3b |
| 60 | t-Bu | 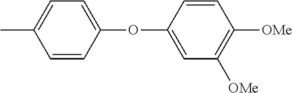 | 126- 129 | 0.25 | 30% EtOAc/ 70% hexane | 412 (M + H)+ [FAB] | B3e, C2b |
| 61 | t-Bu | 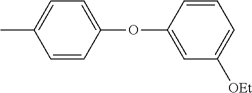 | 201- 204 | 0.25 | 10% EtOAc/ 90% hexane | 396 (M + H)+ [FAB] | B3a, C2b, D2 |
| 62 | t-Bu | 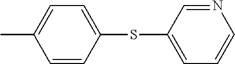 | 163- 164 | 0.30 | 40% EtOAc/ 60% hexane | 369 (M + H)+ [FAB] | B4a, C2b |
| 63 | t-Bu | 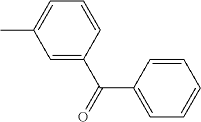 | 162- 163 | 0.20 | 25% EtOAc/ 75% hexane | 363 (M+) [EI] | C2b |
| 64 | t-Bu | 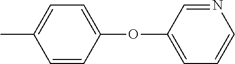 | 127- 129 | 0.22 | 40% EtOAc/ 60% hexane | 353 (M + H)+ [FAB] | B3e Step 1, B2, C2b |
| 65 | t-Bu | 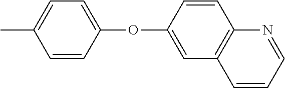 | 85- 87 | 0.20 | 50% EtOAc/ 50% hexane | 402 (M+) [EI] | B3e Step 1, B2, C2b |
| 66 | t-Bu | 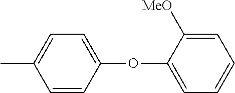 | 108- 110 | 0.25 | 10% EtOAc/ 90% hexane | 381 (M+) [EI] | B3e, C2b |
| 67 | t-Bu | 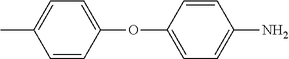 | 186- 189 | 0.25 | 30% EtOAc/ 70% hexane | 367 (M + H)+ [FAB] | B6, C2b, D6 |
| 68 | t-Bu | 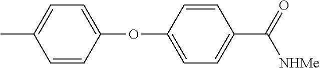 | 221- 224 | 0.25 | 60% EtOAc/ 40% hexane | 409 (M + H)+ [FAB] | B3e, C2b, D5b |
| 69 | t-Bu | 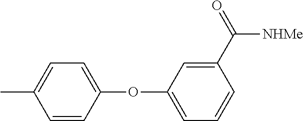 | 114- 117 | 0.25 | 60% EtOAc/ 40% hexane | 409 (M + H)+ [FAB] | B3e, C2b, D5b |
| 70 | t-Bu | 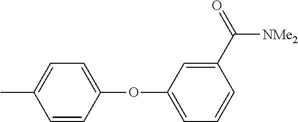 | 201- 203 | 0.25 | 60% EtOAc/ 40% hexane | 423 (M + H)+ [FAB] | B3e, C2b, D5b |
| 71 | t-Bu | 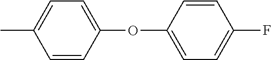 | 148- 151 | 0.25 | 20% EtOAc/ 80% hexane | 370 (M + H)+ [FAB] | B3e, C2b |
| 72 | t-Bu | 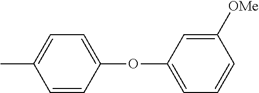 | 188- 201 | 0.25 | 20% EtOAc/ 80% hexane | 382 (M + H)+ [FAB] | B3e, C2b |
| 73 | t-Bu | 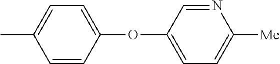 | 134- 136 | 0.25 | 20% EtOAc/ 80% hexane | 367 (M + H)+ [FAB] | B3e, C2b |
| 74 | t-Bu | 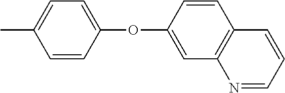 | 176- 178 | 0.25 | 50% EtOAc/ 50% hexane | 403 (M + H)+ [FAB] | B3e, C2b |
| 75 | t-Bu | 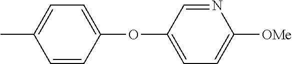 | 132- 134 | 0.52 | 40% EtOAc/ 60% hexane | 383 (M + H)+ [FAB] | B3k, C3b |
| 76 | t-Bu | 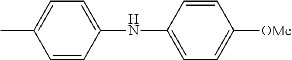 | 160- 162 | 0.79 | 75% EtOAc/ 25% hexane | 381 (M + H)+ [FAB] | C3a |
| 77 | t-Bu | 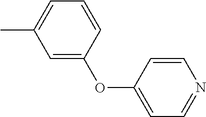 | 140- 143 | 0.25 | 50% EtOAc/ 50% CH2Cl2 | 352 (M+) [EI] | B4b, C3b |
| 78 | t-Bu | 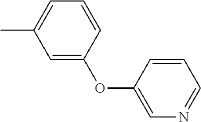 | 147- 150 | 0.25 | 50% EtOAc/ 50% CH2Cl2 | 352 (M+) [EI] | B3f, C3b |
| 79 | t-Bu | 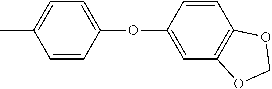 | 166- 170 | 0.44 | 50% EtOAc/ 50% hexane | 396 (M + H)+ [FAB] | C3b |
| 80 | t-Bu | 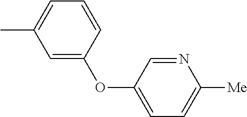 | 190- 193 | 0.25 | 50% EtOAc/ 50% CH2Cl2 | 367 (M + H)+ [FAB] | B3g, C3b |
| 81 | t-Bu | 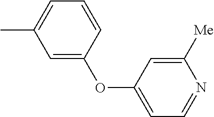 | 136- 140 | 0.25 | 50% EtOAc/ 50% CH2Cl2 | 367 (M + H)+ [FAB] | B4b, C3b |
| 82 | t-Bu | 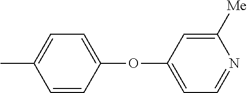 | 65- 67 | 0.25 | 50% EtOAc/ 50% CH2Cl2 | 367 (M + H)+ [FAB] | B4b, C3b |
| 83 | t-Bu | 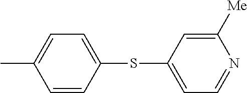 | 68- 72 | 0.25 | 50% EtOAc/ 50% CH2Cl2 | 383 (M + H)+ [FAB] | B4a, C3b |
| 84 | t-Bu | 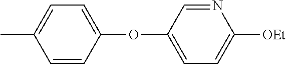 | 146 | 0.49 | 40% EtOAc/ 60% hexane | 397 (M + H)+ [FAB] | B3k C3b, D9 |
| 85 | t-Bu | 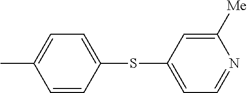 | 164- 165 | 0.25 | 50% EtOAc/ 50% CH2Cl2 | 382 (M+) [EI] | B4a, C3b |
| 86 | t-Bu | 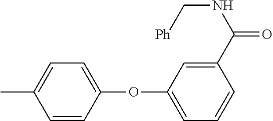 | 175- 177 | 0.25 | 20% EtOAc/ 80% hexane | 485 (M + H)+ [FAB] | B3e, C3b, D5b |
| 87 | t-Bu | 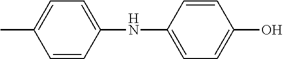 | 137- 141 | 0.30 | 50% EtOAc/ 50% hexane | 366 (M+) [EI] | C3a, D2 step 1 |
| 88 | t-Bu | 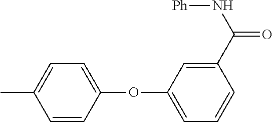 | 120- 122 | 0.25 | 20% EtOAc/ 80% hexane | 471 (M + H)+ [HPLC ES-MS] | B3e, C3b, D5b |
| 89 | t-Bu | 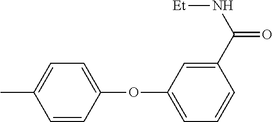 | 168- 170 | 0.25 | 50% EtOAc/ 50% hexane | 423 (M + H)+ [HPLC ES-MS] | B3e, C3b, D5b |
| 90 | t-Bu | 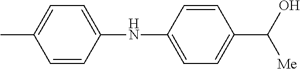 | 80- 85 | 0.25 | 50% EtOAc/ 50% hexane | 396 (M + H)+ [HPLC ES-MS] | B1, C2b, D10 |
| 91 | t-Bu | 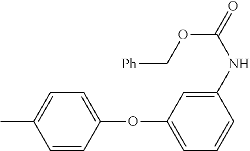 | 73- 75 | 0.25 | 30% EtOAc/ 70% hexane | 501 (M + H)+ [HPLC ES-MS] | B3e, C3b, D11 |
| 92 | t-Bu | 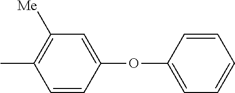 | | 0.50 | 5% acetone/ 95% CH2Cl2 | 366 (M + H)+ [FAB] | B1a |
| 93 | t-Bu | 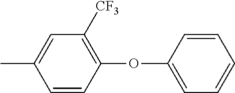 | 199- 200 | 0.59 | 5% acetone/ 95% CH2Cl2 | 419 (M+) [FAB] | B1a |
| 94 | t-Bu | 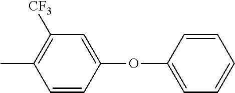 | | 0.59 | 5% acetone/ 95% CH2Cl2 | 419 (M+) [FAB] | B1a |
| 95 | t-Bu | 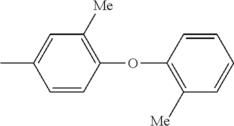 | 78- 82 | 0.25 | 10% EtOAc/ 90% CH2Cl2 | 379 (M+) [EI] | B3e, C3b |
| 96 | t-Bu | 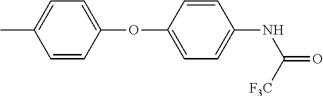 | 214- 217 | 0.75 | 60% EtOAc/ 40% hexane | 463 (M + H)+ [FAB] | C2b, D3 |
| 97 | t-Bu | 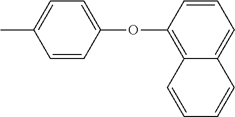 | 235 | 0.35 | 25% EtOAc/ 75% hexane | 402 (M + H) + v | B3b, C2b |
| 98 | t-Bu | 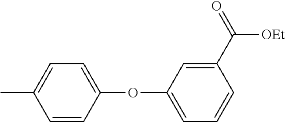 | 153- 155 | 0.25 | 30% EtOAc/ 70% hexane | 424 (M + H)+ [FAB] | B3e, C2b |
| 99 | t-Bu | 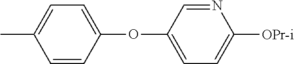 | 100 | 0.62 | 40% EtOAc/ 60% hexane | 411 (M + H)+ [FAB] | B3a, B1, C3b |
| 100 | t-Bu | 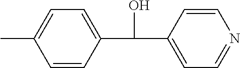 | 110- 115 | 0.15 | 100% EtOAc | 367 (M + H)+ [FAB] | |

| | | | | | | | |
|---|---|---|---|---|---|---|---|
| 101 | t-Bu | 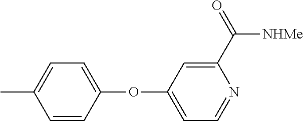 | | 0.50 | 100% EtOAc | 410 (M + H)+ [FAB] | B10, B4b, C2b |
| 102 | t-Bu | 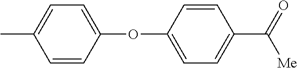 | 153- 155 | | | 395 (M + H)+ [FAB] | C3b |
| 103 | t-Bu | 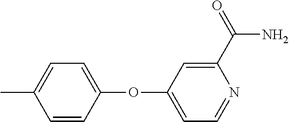 | | 0.52 | 100% EtOAc | 396 (M + H)+ [HPLC ES-MS] | B10, B4b, C2b |
| 104 | t-Bu | 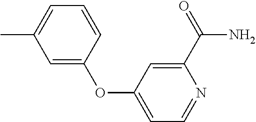 | | 0.75 | 100% EtOAc | 396 (M + H)+ [HPLC ES-MS] | B10, B4b, C2b |
| 105 | t-Bu | 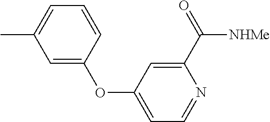 | 107- 110 | 0.85 | 100% EtOAc | 410 (M + H)+ [FAB] | B10, B4b, C2b |
| 106 | t-Bu | 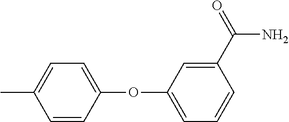 | 132- 135 | | | | B3d step 2, C3a |
| 107 | t-Bu | 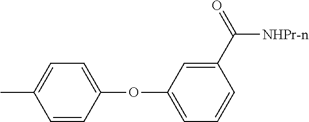 | | 0.58 | 100% EtOAc | | C3a, D5b |
| 108 | t-Bu | 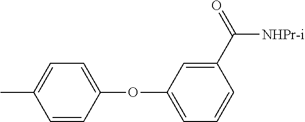 | | 0.58 | 100% EtOAc | | C3a, D5b |
| 109 | t-Bu | 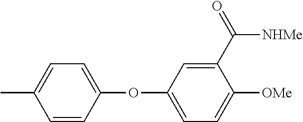 | 137- 140 | 0.62 | 100% EtOAc | 439 (M + H)+ [HPLC ES-MS] | B3a step 1, B12, D5b step 2, C3a |
| 110 | t-Bu | 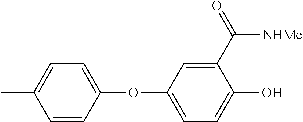 | 163- 166 | 0.73 | 100% EtOAc | 425 (M + H)+ [HPLC ES-MS] | B3a step 1, B12, D5b step 2, C3a |
| 111 | t-Bu | 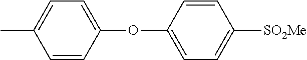 | 180- 181 | | | | B3b step 1, B11, B3b step 2, C2a |
| 112 | t-Bu | 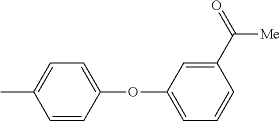 | 135- 139 | | | | B3b, C2a |
| 113 | t-Bu | 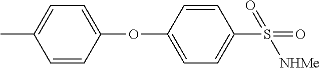 | 212- 215 | | | | B3d step 2a, C2a |
| 114 | t-Bu | 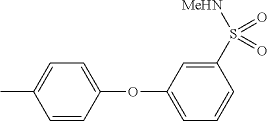 | 98- 100 | | | | B3d step 2, C2a |
| 115 | t-Bu | 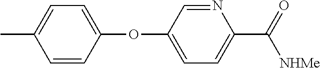 | 135- 138 | | | | B10, B4b, C2a |
| 116 | t-Bu | 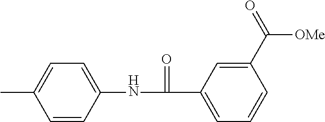 | 219- 221 | 0.78 | 80% EtOAc/ hexane | 437 (M + H)+ [HPLC ES-MS] | C3a, D5b, step 2 |
| 117 | t-Bu | 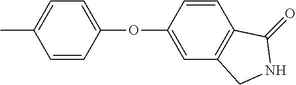 | 160- 164 | | | | B3a step 1, B3d step 2, C3a |
| 118 | t-Bu | 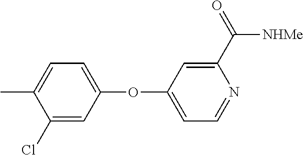 | 124 | 0.39 | 5% MeOH/ 45% EtOAc/ 50% hexane | | C1c, D5b |
| 119 | t-Bu | 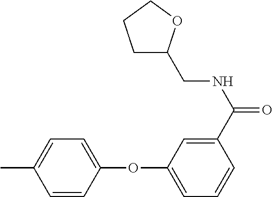 | 73- 75 | 0.41 | 100% EtOAc | 479 (M + H)+ [HPLC ES-MS] | B3a, C4a, D5b |
| 120 | t-Bu | 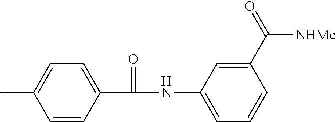 | | 0.32 | 100% EtOAc | 436 (M + H)+ [HPLC ES-MS] | C1b, D5b step 1, step 2 |
| 121 | t-Bu | 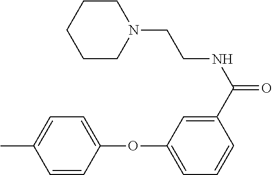 | | 0.23 | 10% MeOH/ 90% CH2Cl2 | 506 (M + H)+ [HPLC ES-MS] | B3a, C4a, D5b |
| 122 | t-Bu | 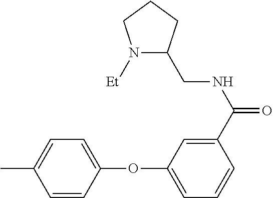 | | 0.18 | 10% MeOH/ 90% CH2Cl2 | 506 (M + H)+ [HPLC ES-MS] | B3a, C4a, D5b |
| 123 | t-Bu | 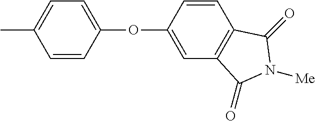 | 229- 231 | 0.37 | 40% EtOAc/ 60% hexane | 435 (M + H)+ [HPLC ES-MS] | D5b step 1, B3b step 2, C3a |
| 124 | t-Bu | 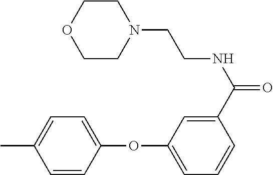 | | 0.21 | 5% MeOH/ 95% CH2Cl2 | 508 (M + H)+ [HPLC ES-MS] | B3a, C4a, D5b |
| 125 | t-Bu | 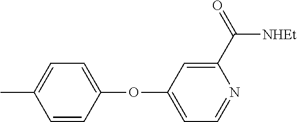 | 167- 170 | 0.34 | 5% MeOH/ 45% EtOAc/ 50% hexane | 424 (M + H)+ [HPLC ES-MS] | C3b, D5b |
| 126 | t-Bu | 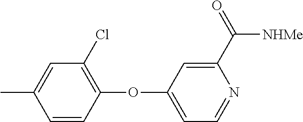 | 124 | 0.26 | 5% MeOH/ 45% EtOAc/ 50% hexane | | C3b, D5b |
| 127 | t-Bu | 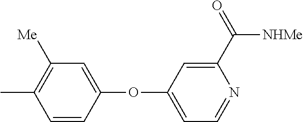 | 125- 128 | 0.28 | 5% MeOH/ 45% EtOAc/ 50% hexane | | C3b, D5b |
| 128 | t-Bu | 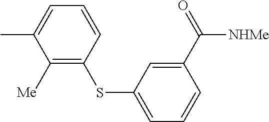 | | 0.37 | 50% EtOAc/ 50% pet ether | 426 (M + H)+ [HPLC ES-MS] | C3b |
| 129 | t-Bu | 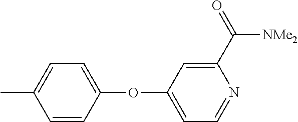 | | 0.10 | 50% EtOAc/ 50% pet ether | 424 (M + H)+ [HPLC ES-MS] | C3b |
| 130 | t-Bu | 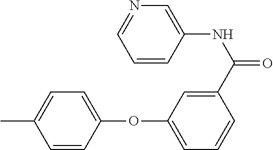 | | 0.18 | 70% EtOAc/ 30% hexane | 472 (M + H)+ [HPLC ES-MS] | D5b step 2 |
| 131 | t-Bu | 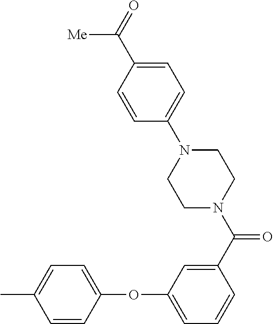 | | 0.32 | | 582 (M + H)+ [HPLC ES-MS] | C3b |
| 132 | t-Bu | 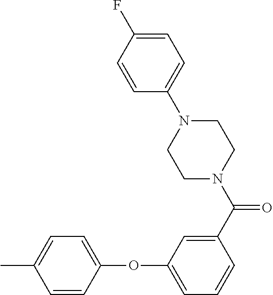 | | 0.57 | | 558 (M + H)+ [HPLC ES-MS] | C3b |
| 133 | t-Bu | 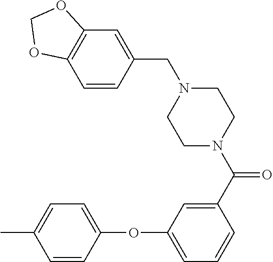 | | 0.21 | | 598 (M + H)+ [HPLC ES-MS] | C3b |
| 134 | t-Bu | 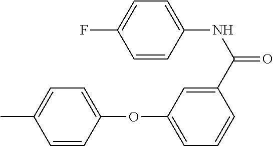 | | 0.86 | | 489 (M + H)+ [HPLC ES-MS] | C3b |
| 135 | t-Bu | 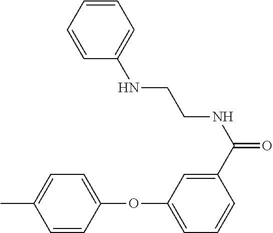 | | 0.64 | | 514 (M + H)+ [HPLC ES-MS] | C3b |
| 136 | t-Bu | 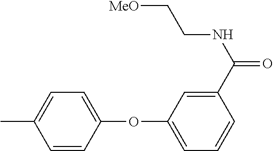 | | 0.29 | | 453 (M + H)+ [HPLC ES-MS] | C3b |
| 137 | t-Bu | 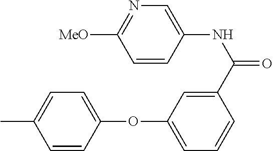 | | 0.70 | | 502 (M + H)+ [HPLC ES-MS] | C3b |
| 138 | t-Bu | 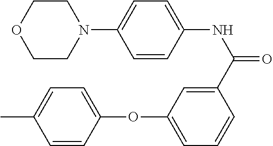 | | 0.50 | | 556 (M + H)+ [HPLC ES-MS] | C3b |
| 139 | t-Bu | 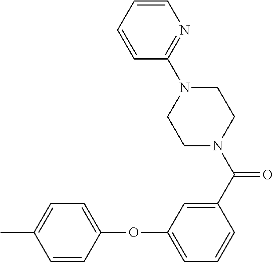 | | 0.27 | | 541 (M + H)+ [HPLC ES-MS] | C3b |
| 140 | t-Bu | 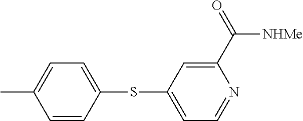 | 211- 212 | 0.27 | 50% EtOAc/ 50% pet ether | 426 (M + H)+ [HPLC ES-MS] | C3b |
| 141 | t-Bu | 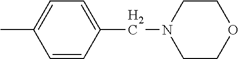 | 195- 198 | | | | B8, C2a |
| 142 | t-Bu | 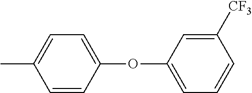 | 170- 171 | | | | C3a |
| 143 | t-Bu | 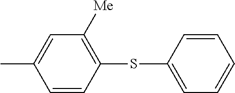 | 141- 144 | 0.63 | 5% acetone/ 95% CH2Cl2 | 382 (M + H)+ [FAB] | B3b, step 1, 2, C1d |
| 144 | t-Bu | 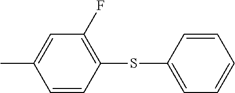 | | 0.57 | 5% acetone/ 95% CH2Cl2 | 386 (M + H)+ [FAB] | B3b, step 1, 2, C1d |
| 145 | t-Bu | 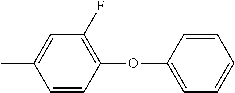 | 145- 148 | 0.44 | 5% acetone/ 95% CH2Cl2 | 370 (M + H)+ [FAB] | B3b, step 1, 2, C1d |
| 146 | t-Bu | 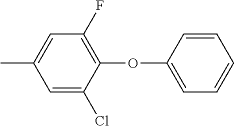 | 197- 202 | 0.50 | 5% acetone/ 95% CH2Cl2 | 404 (M + H)+ [FAB] | B3b, step 1, 2, C1d |
| 147 | t-Bu | 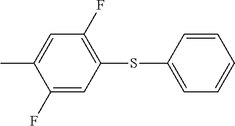 | | 0.60 | 5% acetone/ 95% CH2Cl2 | 404 (M + H)+ [FAB] | B3b, step 1, 2, C1d |
| 148 | t-Bu | 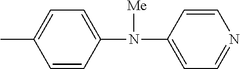 | 126- 129 | 0.17 | 30% MeOH/ 70% EtOAc | 366 (M + H)+ [FAB] | B4c, C4a |
| 149 | t-Bu | 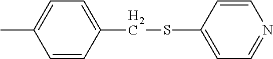 | | | | 383 (M + H)+ [HPLC ES-MS] | C3b |
| 150 | t-Bu | 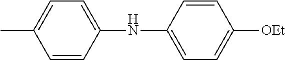 | 156- 159 | 0.48 | 40% EtOAc/ hexane | 395 (M + H)+ [HPLC ES-MS] | C3a, D2 step 1, step 2 |
| 151 | t-Bu | 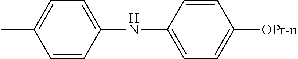 | 157- 159 | 0.51 | | 409 (M + H)+ [HPLC ES-MS] | C3a, D9 step 1, step 2 |
| 152 | t-Bu | 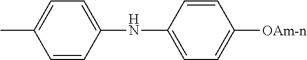 | 130- 132 | 0.60 | | 437 (M + H)+ [HPLC ES-MS] | C3a, D9 step 1, step 2 |
| 153 | t-Bu | 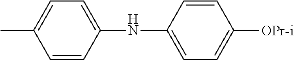 | 146- 150 | 0.54 | 40% EtOAc/ hexane | 409 (M + H)+ [HPLC ES-MS] | C3a, D2 step 1, step 2 |
| 154 | t-Bu | 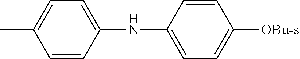 | 145- 148 | 0.57 | 40% EtOAc/ hexane | 423 (M + H)+ [HPLC ES-MS] | C3a, D2 step 1, step 2 |
| 155 | t-Bu | 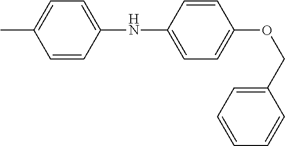 | 175- 178 | 0.51 | 40% EtOAc/ hexane | 457 (M + H)+ [HPLC ES-MS] | C3a, D2 step 1, step 2 |
| 156 | t-Bu | 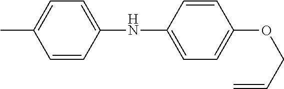 | 149- 152 | 0.48 | 40% EtOAc/ hexane | 407 (M + H)+ [HPLC ES-MS] | C3a, D1 step 1, step 2 |
| 157 | t-Bu | 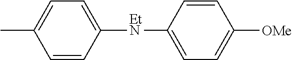 | 146- 147 | 0.36 | 40% EtOAc/ hexane | 409 (M + H)+ [HPLC ES-MS] | C3a |
| 158 | t-Bu | 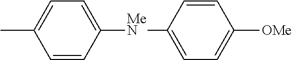 | 156- 158 | 0.43 | 40% EtOAc/ hexane | 395 (M + H)+ [FAB] | C3a |
| 159 | t-Bu | 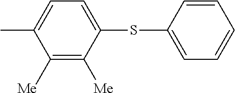 | 164- 168 | 0.52 | 5% acetone/ 95% CH2Cl2 | 396 (M + H)+ [HPLC ES-MS] | B3b, step 1, 2, C1d |
| 160 | t-Bu | 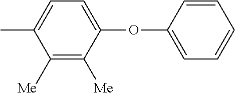 | | 0.36 | 5% acetone/ 95% CH2Cl2 | 380 (M + H)+ [FAB] | B3b, step 1, 2, C1d |
| 161 | t-Bu | 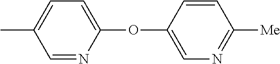 | 169- 171 | | | 368 (M + H)+ [FAB] | C3b |
| 162 | t-Bu | 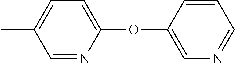 | 168 | 0.11 | 50% EtOAc/ 50% pet ether | | C3b |
| 163 | t-Bu | 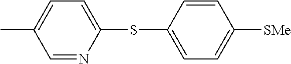 | 146 | | | | C3b |
| 164 | t-Bu | 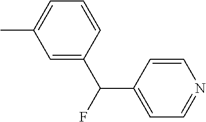 | | 0.45 | 100% EtOAc | 369 (M + H)+ [FAB] | C2b |
| 165 | t-Bu | 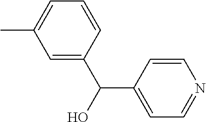 | | 0.20 | 100% EtOAc | 367 (M + H)+ [FAB] | B9, C2b |
| 166 | t-Bu | 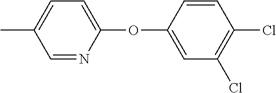 | 187- 188 | 0.46 | 30% EtOAc/ hexane | 421 (M + H)+ [FAB] | C3b |
| 167 | t-Bu | 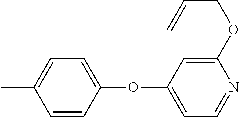 | 133 | 0.36 | | 409 (M + H)+ [FAB] | C3a, D9 step 1, step 2 |
| 168 | t-Bu | 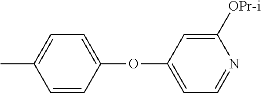 | | 0.39 | 40% EtOAc/ 60% hexane | 411 (M + H)+ [FAB] | C3a, D9 step 1, step 2 |
| 169 | t-Bu | 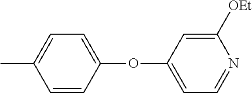 | | 0.32 | 5% acetone/ 95% CH2Cl2 | 397 (M + H)+ [HPLC ES-MS] | B3k, C8 |
| 170 | t-Bu | 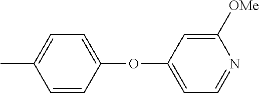 | | 0.21 | 5% acetone/ 95% CH2Cl2 | 383 (M + H)+ [HPLC ES-MS] | B3k, C8 |
| 171 | t-Bu | 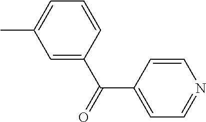 | | 0.60 | 100% EtOAc | 365 (M + H)+ [FAB] | C2b |
| 172 | t-Bu | 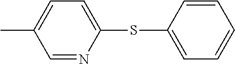 | | 0.16 | 30% EtOAc/ 70% hexane | 369 (M + H)+ [HPLC ES-MS] | C8 |
| 173 | t-Bu | 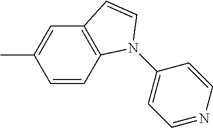 | 125- 129 | 0.09 | 5% MeOH/ 45% EtOAc/ 50% hexane | | C3b |
| 174 | t-Bu | 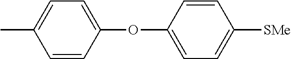 | 147- 149 | | | | B3b, C2a |
| 175 | t-Bu | 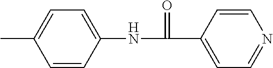 | | 0.30 | 100% EtOAc | 380 (M + H)+ [HPLC ES-MS] | C3a, D5b, step 2 |
| 176 | t-Bu | 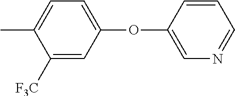 | | 0.50 | 25% EtOAc/ 75% hexane | 353 (M + H)+ [Cl] | MS B4b, C8 |

TABLE 2
3-Substituted 5-Isoxazolyl Ureas

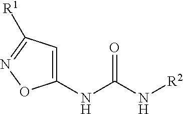

| | | | mp | TLC | Solvent | Mass Spec. | Synth. |
|---|---|---|---|---|---|---|---|
| Entry | R¹ | R² | (° C.) | R_{f} | System | [Source] | Method |
| 177 | Me | 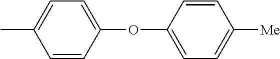 | 169- 170 | 0.25 | 5% acetone/ 95% CH2Cl2 | 324 (M + H)+ [FAB] | C1b |
| 178 | i-Pr | 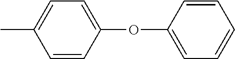 | 153- 156 | 0.54 | 50% EtOAc/ 50% pet ether | 338 (M + H)+ [FAB] | C1b |
| 179 | i-Pr | 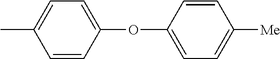 | 166- 170 | 0.54 | 50% EtOAc/ 50% pet ether | 352 (M + H)+ [FAB] | C1b |
| 180 | i-Pr | 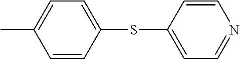 | 112- 117 | 0.29 | 5% MeOH/ 95% CH2Cl2 | 355 (M + H)+ [FAB] | A2, B4a, C3a |
| 181 | i-Pr | 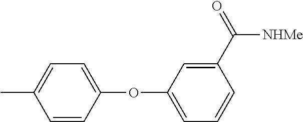 | | 0.08 | 50% EtOAc/ 50% hexane | 395 (M + H)+ [HPLC ES-MS] | C8 |
| 182 | i-Pr | 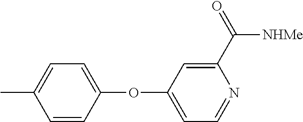 | 169- 170 | 0.20 | 50% EtOAc/ 50% pet ether | 396 (M + H)+ [HPLC ES-MS] | C3b |
| 183 | i-Pr | 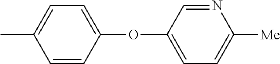 | | 0.10 | 50% EtOAc/ 50% hexane | 353 (M + H)+ [HPLC ES-MS] | C8 |
| 184 | i-Pr | 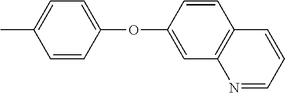 | | 0.09 | 50% EtOAc/ 50% hexane | 389 (M + H)+ [HPLC ES-MS] | C8 |
| 185 | i-Pr | 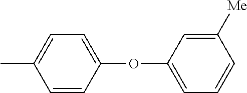 | | 0.23 | 30% EtOAc/ 70% hexane | 352 (M + H)+ [HPLC ES-MS] | C8 |
| 186 | i-Pr | 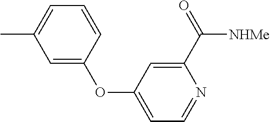 | 194- 195 | 0.29 | 50% EtOAc/ 50% pet ether | 396 (M + H)+ [HPLC ES-MS] | C3b |
| 187 | 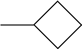 | 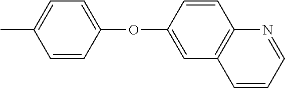 | | 0.03 | 50% EtOAc/ 50% hexane | 401 (M + H)+ [FAB] | C8 |
| 188 | 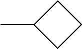 | 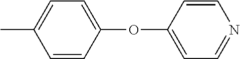 | | | | 351 (M + H)+ [HPLC ES-MS] | C8 |
| 189 | 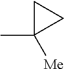 | 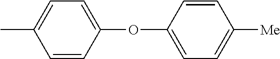 | 175- 178 | 0.43 | 50% EtOAc/ 50% pet ether | 364 (M + H)+ [FAB] | C1b |
| 190 | t-Bu | 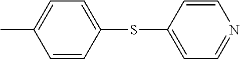 | | 0.21 | 5% MeOH/ 95% CH2Cl2 | 369 (M + H)+ [FAB] | B4a, C2a |
| 191 | t-Bu | 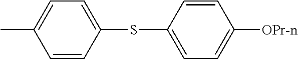 | | 0.52 | 50% EtOAc/ 50% hexane | 426 (M + H)+ [FAB] | B5, C4a |
| 192 | t-Bu | 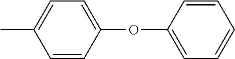 | 182- 184 | | | 352 (M + H)+ [FAB] | C1b |
| 193 | t-Bu | 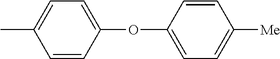 | 165 dec | 0.34 | 60% EtOAc/ 40% pet ether | 366 (M + H)+ [FAB] | C1b |
| 194 | t-Bu | 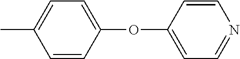 | 210 dec | 0.05 | 5% acetone/ 95% CH2Cl2 | 353 (M + H)+ [FAB] | C3a |
| 195 | t-Bu | 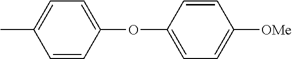 | 174- 175 | 0.25 | 5% acetone/ 95% CH2Cl2 | 382 (M + H)+ [FAB] | C3a |
| 196 | t-Bu | 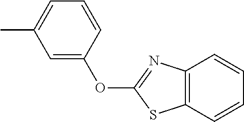 | 90-92 | 0.16 | 5% acetone/ 95% CH2Cl2 | 409 (M + H)+ [FAB] | C2a |
| 197 | t-Bu | 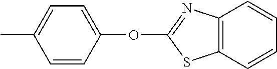 | 221 dec | 0.14 | 5% acetone/ 95% CH2Cl2 | 409 (M + H)+ [FAB] | C2a |
| 198 | t-Bu | 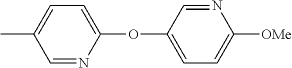 | 196- 198 | 0.17 | 5% MeOH/ 95% CH2Cl2 | 368 (M + H)+ [FAB] | A2, B3b, C3a |
| 199 | t-Bu | 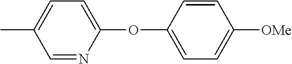 | 204- 206 | 0.27 | 50% EtOAc/ 50% pet ether | 383 (M + H)+ [FAB] | A2, B3a, C3a |
| 200 | t-Bu | 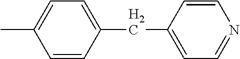 | 179- 180 | | | 351 (M + H)+ [FAB] | A2, C3a |
| 201 | t-Bu | 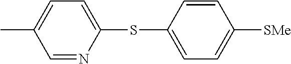 | | 0.33 | 50% EtOAc/ 50% pet ether | 414 (M+) (EI) | A2, B4a, C3a |
| 202 | t-Bu | 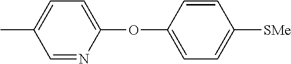 | 188- 189 | 0.49 | 50% EtOAc/ 50% pet ether | 399 (M + H)+ [HPLC ES-MS] | A2, B4a, C3a |
| 203 | t-Bu | 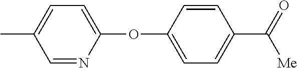 | 179- 180 | 0.14 | 5% MeOH/ 95% CH2Cl2 | 395 (M + H)+ [FAB] | A2, B4a, C3a |
| 204 | t-Bu | 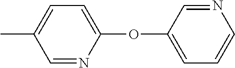 | 197- 199 | 0.08 | 10% acetone/ 90% CH2Cl2 | 353 (M + H)+ [FAB] | A2, B3h, C3a |
| 205 | t-Bu | 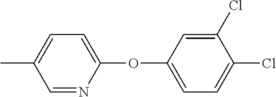 | 136- 139 | 0.33 | 50% EtOAc/ 50% pet ether | 421 (M + H)+ [FAB] | A2, B3b, C3a |
| 206 | t-Bu | 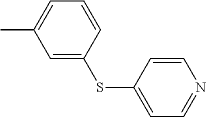 | 213 dec | 0.05 | 5% acetone/ 95% CH2Cl2 | 369 (M + H)+ [FAB] | C3a |
| 207 | t-Bu | 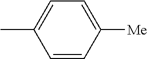 | | 0.60 | 5% MeOH/ 95% CH2Cl2 | 274 (M + H)+ [FAB] | C2a |
| 208 | t-Bu | 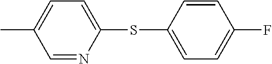 | 118- 121 | 0.19 | 5% MeOH/ 95% CH2Cl2 | 387 (M + H)+ [FAB] | A2, B4a, C3a |
| 209 | t-Bu | 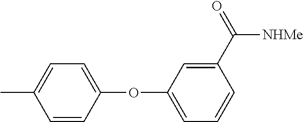 | 217- 219 | 0.18 | 5% MeOH/ 95% CHCl3 | | A2, C3b |
| 210 | t-Bu | 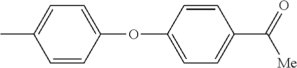 | | 0.48 | 50% EtOAc/ 50% hexane | 394 (M + H)+ [HPLC ES-MS] | C8 |
| 211 | t-Bu | 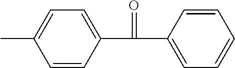 | | 0.17 | 30% EtOAc/ 70% hexane | 364 (M + H)+ [HPLC ES-MS] | C8 |
| 212 | t-Bu | 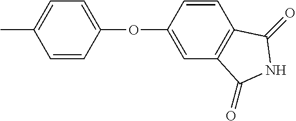 | | 0.79 | 70% EtOAc/ 30% hexane | 421 (M + H)+ [HPLC ES-MS] | B3a step 1, B3d step 2, C3a |
| 213 | t-Bu | 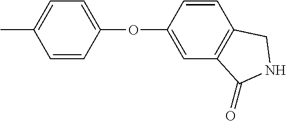 | | 0.50 | 50% EtOAc/ 50% hexane | 407 (M + H)+ [HPLC ES-MS] | B3a step 1, B3d step 2, C3a |
| 214 | t-Bu | 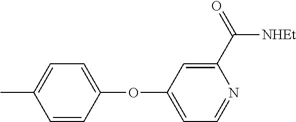 | 182- 185 | 0.25 | 5% MeOH/ 45% EtOAc/ 50% hexane | 424 (M + H)+ [HPLC ES-MS] | C3b, D5b |
| 215 | t-Bu | 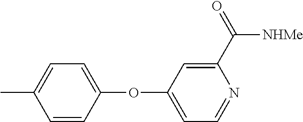 | 198- 200 | 0.20 | 5% MeOH/ 45% EtOAc/ 50% hexane | 444 (M + H)+ [HPLC ES-MS] | C3b, D5b |
| 216 | t-Bu | 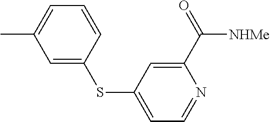 | | 0.24 | 50% EtOAc/ 50% pet ether | 426 (M + H)+ [HPLC ES-MS] | C3b |
| 217 | t-Bu | 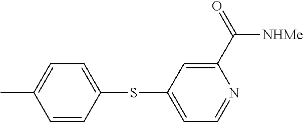 | 215- 217 | | | 426 (M + H)+ [HPLC ES-MS] | C3b |
| 218 | t-Bu | 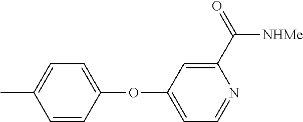 | 188- 200 | 0.22 | 50% EtOAc/ 50% pet ether | 410 (M + H)+ [HPLC ES-MS] | C3b |
| 219 | t-Bu | 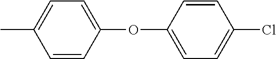 | 214- 215 | 0.35 | 5% acetone/ 95% CH2Cl2 | | A2, C2b |
| 220 | t-Bu | 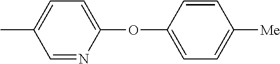 | 180 | | | | C3b |
| 221 | t-Bu | 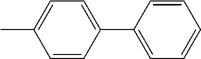 | 160- 162 | 0.58 | 50% EtOAc/ 50% pet ether | 336 (M+) [CI] | C3b |
| 222 | t-Bu | 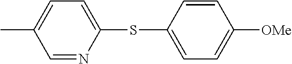 | | 0.18 | 50% EtOAc/ 50% pet ether | | C3b |
| 223 | t-Bu | 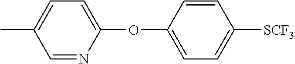 | 163- 165 | 0.21 | 5% MeOH/ 95% CH2Cl2 | 453 (M + H)+ [HPLC ES-MS] | C3b |
| 224 | t-Bu | 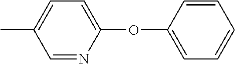 | 208- 212 | 0.17 | 5% MeOH/ 95% CH2Cl2 | 353 (M + H)+ [FAB] | C3b |
| 225 | t-Bu | 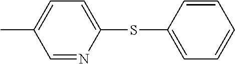 | 109- 112 | 0.17 | 5% MeOH/ 95% CH2Cl2 | 369 (M + H)+ [FAB] | C3b |
| 226 | t-Bu | 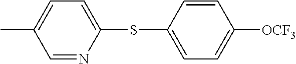 | 155-156 | 0.57 | 10% MeOH/ CH2Cl2 | 453 (M + H)+ [FAB] | C3b |
| 227 | t-Bu | 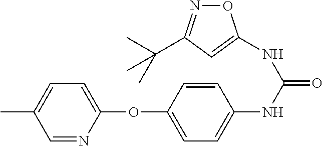 | 231- 234 | 0.54 | 10% MeOH/ CH2Cl2 | 534 (M + H)+ [FAB] | C3b |
| 228 | t-Bu | 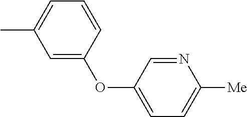 | 179- 180 | 0.24 | 5% MeOH/ 95% CHCl3 | | A2, C3b |
| 229 | t-Bu | 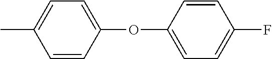 | | 0.30 | 5% MeOH/ 95% CHCl3 | 370 (M + H)+ [FAB] | A2, C3b |
| 230 | t-Bu | 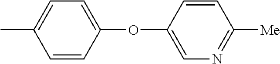 | 178- 180 | 0.20 | 5% MeOH/ 95% CHCl3 | | A2, C3b |
| 231 | t-Bu | 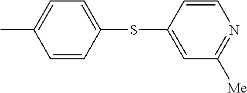 | 186- 187 | 0.20 | 5% MeOH/ 95% CHCl3 | | A2, C3b |
| 232 | t-Bu | 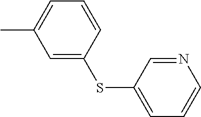 | 149- 152 | 0.28 | 5% MeOH/ 95% CHCl3 | | A2, C3b |
| 233 | t-Bu | 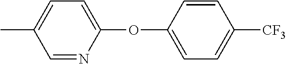 | 210- 213 | 0.06 | 10% MeOH/ CH2Cl2 | 421 (M + H)+ [FAB] | C3b |
| 234 | t-Bu | 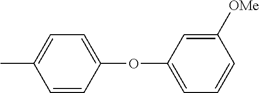 | 132-133 | 0.43 | 5% MeOH/ 95% CHCl3 | | A2, C3b |
| 235 | t-Bu | 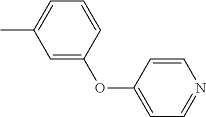 | 71-73 | 0.27 | 5% MeOH/ 95% CHCl3 | | A2, C3b |
| 236 | t-Bu | 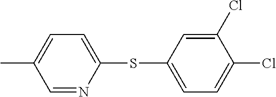 | 176- 177 | 0.44 | 10% MeOH/ Ch2Cl2 | 437 (M + H)+ [FAB] | C3b |
| 237 | t-Bu | 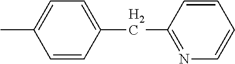 | | 0.09 | 50% EtOAc/ 50% hexane | 351 (M + H)+ [HPLC ES-MS] | C8 |
| 238 | t-Bu | 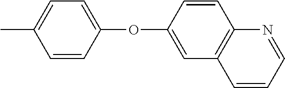 | | 0.16 | 50% EtOAc/ 50% hexane | 403 (M + H)+ [HPLC ES-MS] | C8 |
| 239 | t-Bu | 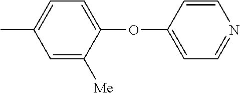 | | 0.15 | 50% EtOAc/ 50% hexane | 381 (M + H)+ [HPLC ES-MS] | C8 |
| 240 | t-Bu | 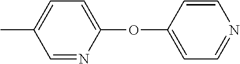 | 215- 216 | 0.19 | 100% EtOAc | 370 (M + H)+ [HPLC ES-MS] | C3b |
| 241 | t-Bu | 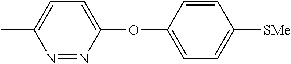 | | 0.42 | 5% MeOH/ 95% CH2Cl2 | | |
| 242 | t-Bu | 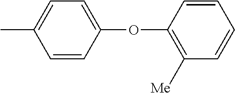 | | 0.74 | 100% EtOAc | 366 (M + H)+ [HPLC ES-MS] | B4b, C8 |
| 243 | t-Bu | 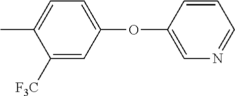 | | 0.12 | 30% EtOAc/ 70% hexane | 421 (M + H)+ [HPLC ES-MS] | C8 |
| 245 | t-Bu | 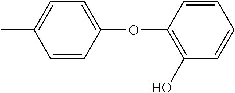 | | 0.68 | 100% EtOAc | 368 (M + H)+ [HPLC ES-MS] | B4b, C8 |
| 246 | t-Bu | 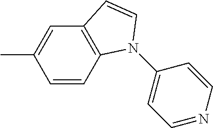 | 142- 144 | 0.13 | 5% MeOH/ 45% EtOAc/ 50% hexane | | A2, C3b |
| 247 | t-Bu | 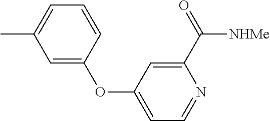 | 205- 207 | 0.31 | 50% EtOAc/ 50% pet ether | 410 (M + H)+ [HPLC ES-MS] | C3b |
| 248 | 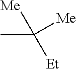 | 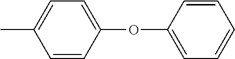 | 154- 155 | 0.50 | 50% EtOAc/ 50% pet ether | 365 (M+) [EI] | C1b |
| 249 | 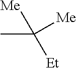 | 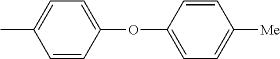 | 160- 162 | 0.37 | 5% acetone/ 95% CH2Cl2 | 380 (M + H)+ [FAB] | C1b |
| 250 | 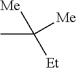 | 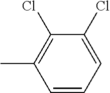 | 196- 199 | 0.58 | 5% acetone/ 95% CH2Cl2 | 342 (M + H)+ [FAB] | C1b |
| 251 | 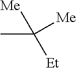 | 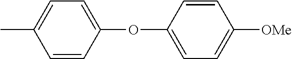 | 137- 138 | 0.25 | 5% acetone/ 95% CH2Cl2 | 396 (M + H)+ [FAB] | A2, B3a, C3a |
| 252 | 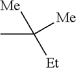 | 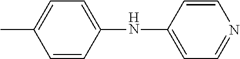 | | 0.18 | 5% MeOH/ CHCl3 | 364 (M+) [EI] | A2, C3a |
| 253 | 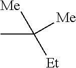 | 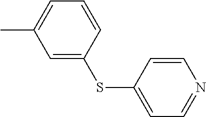 | 215- 221 dec | | | 383 (M + H)+ [FAB] | A2, B4a, C3a |
| 254 | 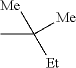 | 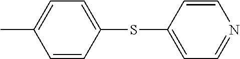 | 187- 188 | 0.42 | 10% MeOH/ CHCl3 | 383 (M + H)+ [FAB] | A2, B4a, C3a |
| 255 | 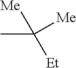 | 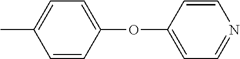 | 90-92 | 0.19 | 30% EtOAc/ 70% pet ether | 366 (M+) [EI] | A2, C3a |
| 257 | 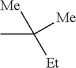 | 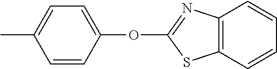 | 199- 200 | 0.33 | 70% EtOAc/ 30% pet ether | 423 (M + H)+ [FAB] | A2, B3a, C3a |
| 258 | 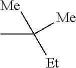 | 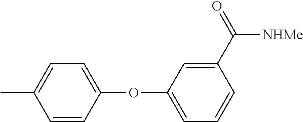 | 117- 119 | 0.14 | 5% MeOH/ 95% CHCl3 | | A2, C3b |
| 259 | 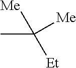 | 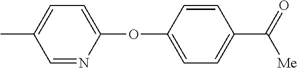 | | 0.37 | 75% EtOAc/ 25% hexane | 409 (M + H)+ [HPLC ES-MS] | C8 |
| 260 | 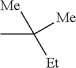 | 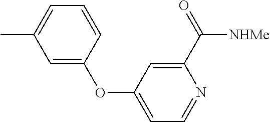 | 194- 195 | 0.25 | 50% EtOAc/ 50% pet ether | 424 (M + H)+ [HPLC ES-MS] | C3b |
| 261 | 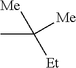 | 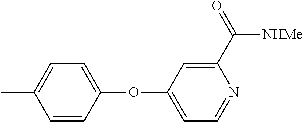 | 216- 217 | 0.20 | 50% EtOAc/ 50% pet ether | 424 (M + H)+ [HPLC ES-MS] | C3b |
| 262 | 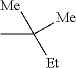 | 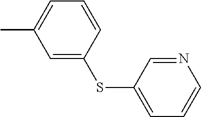 | 62-65 | 0.18 | 5% MeOH/ 95% CHCl3 | | A2, C3b |
| 263 | 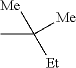 | 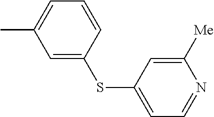 | 86-89 | 0.16 | 5% MeOH/ 95% CHCl3 | | A2, C3b |
| 264 | 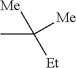 | 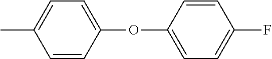 | 145- 146 | 0.32 | 5% MeOH/ 95% CHCl3 | | A2, C3b |
| 265 | 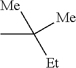 | 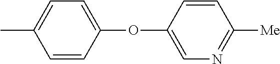 | | 0.23 | 5% MeOH/ 95% CHCl3 | 381 (M + H)+ [FAB] | A2, C3b |
| 266 | 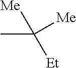 | 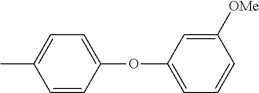 | 0.20 | 5% acetone/ 95% CH2Cl2 | 396 (M + H)+ [FAB] | A2, C3b | |
| 267 | 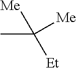 | 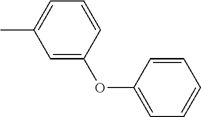 | | 0.38 | 50% EtOAc/ 50% hexane | 366 (M + H)+ [HPLC ES-MS] | C8 |
| 268 | 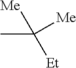 | 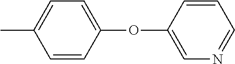 | | 0.14 | 50% EtOAc/ 50% hexane | 367 (M + H)+ (HPLC ES-MS] | C8 |
| 269 | 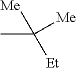 | 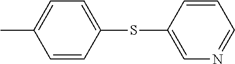 | | 0.21 | 50% EtOAc/ 50% hexane | 383 (M + H)+ [HPLC ES-MS] | C8 |
| 270 | 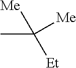 | 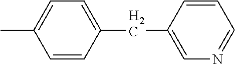 | | 0.10 | 50% EtOAc/ 50% hexane | 365 (M + H)+ [HPLC ES-MS] | C8 |
| 271 | 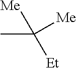 | 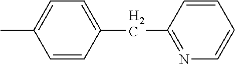 | | 0.14 | 50% EtOAc/ 50% hexane | 365 (M + H)+ [HPLC ES-MS] | C8 |
| 272 | 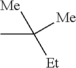 | 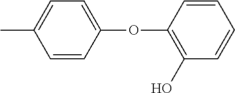 | | 0.35 | 50% EtOAc/ 50% hexane | 382 (M + H)+ [HPLC ES-MS] | C8 |
| 273 | 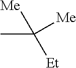 | 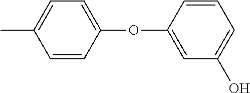 | | 0.48 | 50% EtOAc/ 50% hexane | 382 (M + H)+ [HPLC ES-MS] | C8 |
| 274 | 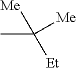 | 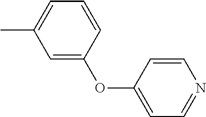 | | 0.20 | 100% EtOAc | 367 (M + H)+ [HPLC ES-MS] | B4b, C8 |
| 275 | 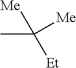 | 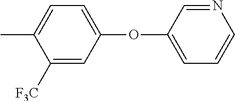 | | 0.56 | 100% EtOAc | 435 (M + H)+ [HPLC ES-MS] | B4b, C8 |
| 276 | 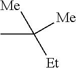 | 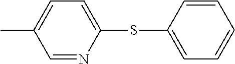 | | 0.57 | 75% EtOAc/ 25% hexane | 383 (M + H)+ [HPLC ES-MS] | C8 |
| 277 | 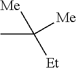 | 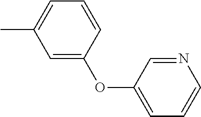 | | 0.40 | 100% EtOAc | | B3f, C8 |
| 278 | 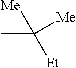 | 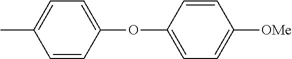 | 63-65 | | | 410 (M + H)+ [FAB] | A2, C3a |
| 279 | 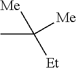 | 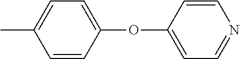 | 84 | 0.16 | 5% MeOH/ 95% CHCl3 | 381 (M + H)+ [FAB] | A2, C3a |
| 280 | 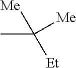 | 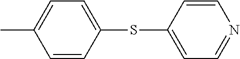 | 189- 192 | 0.16 | 5% MeOH/ 95% CHCl3 | 397 (M + H)+ [HPLC ES-MS] | A2, B4a, C3a |
| 281 | 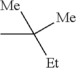 | 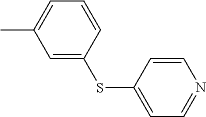 | 189- 191 | 0.17 | 5% MeOH/ 95% CHCl3 | 397 (M + H)+ [FAB] | A2, B4a, C3a |
| 282 | 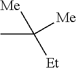 | 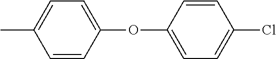 | 123- 125 | | | 414 (M + H)+ [FAB] | A2, C3a |
| 283 | 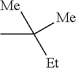 | 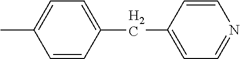 | 175- 177 | 0.16 | 5% MeOH/ 95% CHCl3 | 379 (M + H)+ [FAB] | A2, C3a |
| 284 | 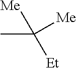 | 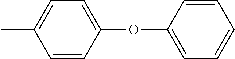 | 135- 137 | 0.33 | 5% MeOH/ 95% CHCl3 | | A2, C3b |
| 285 | 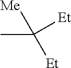 | 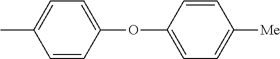 | 67 | 0.41 | 5% MeOH/ 95% CHCl3 | | A2, C3b |
| 286 | 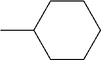 | 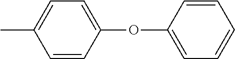 | 155- 156 | 0.38 | 50% EtOAc/ 50% pet ether | 377 (M+) [EI] | C1b |
| 287 | 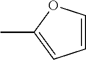 | 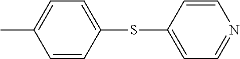 | | 0.18 | 5% MeOH/ 95% CHCl3 | 379 (M + )+ [FAB] | A2, C3b |

TABLE 3
N¹-Substituted-3-tert-butyl-5-pyrazolyl Ureas

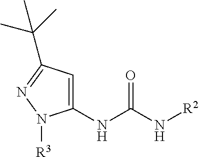

| | | | mp | TLC | Solvent | Mass Spec. | Synth. |
|---|---|---|---|---|---|---|---|
| Ex. | R¹ | R² | (° C.) | R_{f} | System | [Source] | Method |
| 289 | H | 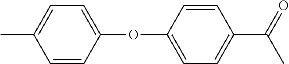 | | 0.07 | 50% EtOAc/ 50% hexane | 393 (M + H)+ [HPLC ES-MS] | C8 |
| 290 | H | 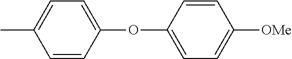 | 181- 183 | | | 381 (M + H)+ [FAB] | C2b |
| 291 | H | 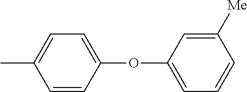 | | 0.30 | 50% EtOAc/ 50% hexane | 365 (M + H)+ [HPLC ES-MS] | C8 |
| 292 | H | 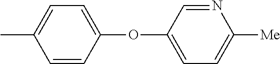 | | | | 366 (M + H)+ [FAB] | C8 |
| 293 | H | 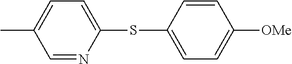 | | 0.53 | 50% EtOAc/ 50% hexane | 398 (M + H)+ [HPLC ES-MS] | C8 |
| 294 | H | 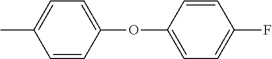 | | | | 369 (M + H)+ [HPLC ES-MS] | C8 |
| 295 | H | 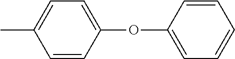 | | 0.27 | 50% EtOAc/ 50% hexane | 351 (M + H)+ [FAB] | C1c |
| 296 | H | 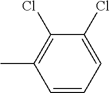 | | 0.59 | 50% EtOAc/ 50% hexane | 327 (M + H)+ [FAB] | C1c |
| 297 | H | 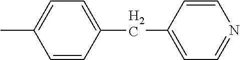 | | 0.30 | 60% acetone/ 40% CH2Cl2 | 350 (M + H)+ [FAB] | C4a |
| 298 | H | 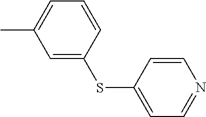 | | 0.07 | 5% MeOH/ 95% CHCl3 | 368 (M + H)+ [FAB] | B4a, C4a |
| 299 | H | 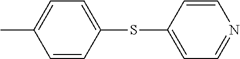 | | 0.18 | 5% MeOH/ 95% CHCl3 | 367 (M+) [EI] | B4a, C4a |
| 300 | H | 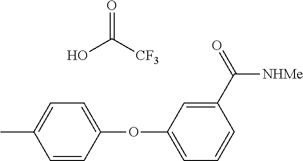 | 160- 161 | | | 408 (M + H)+ [FAB] | A5, B6, C3b isolated at TFA salt |
| 301 | H | 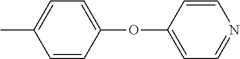 | 228- 232 dec | 0.24 | 10% MeOH/ CHCl3 | 351 (M+) [EI] | C3a |
| 302 | H | 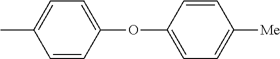 | 204 | 0.06 | 5% acetone/ 95% CH2Cl2 | 364 (M+) [EI] | C3b |
| 303 | H | 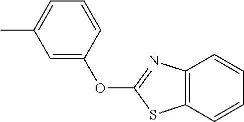 | 110- 111 | 0.05 | 5% acetone/ 95% CH2Cl2 | 408 (M + H+) | C3b |
| 304 | Me | 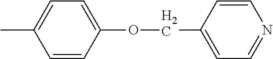 | | 0.10 | 20% acetone/ 80% CH2Cl2 | 380 (M + H)+ [FAB] | C4a |
| 305 | Me | 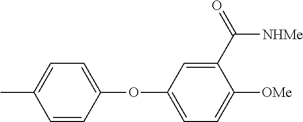 | 99- 101 | 0.19 | 100% EtOAc | 452 (M + H)+ [HPLC ES-MS] | B3a step 1, B12, D5b step 2, C3a |
| 306 | Me | 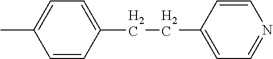 | | 0.48 | 30% acetone/ 70% CH2Cl2 | 378 (M + H)+ [FAB] | B1, C3a |
| 307 | Me | 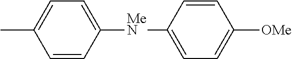 | 135- 137 | 0.03 | 30% EtOAc/ 70% hexane | 408 (M + H)+ [HPLC ES-MS] | C3a |
| 308 | Me | 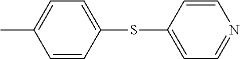 | | 0.35 | 70% acetone/ 30% CH2Cl2 | 382 (M + H)+ [FAB] | B4a, C4a |
| 309 | Me | 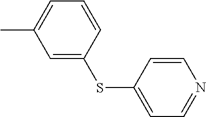 | | 0.46 | 70% acetone/ 30% CH2Cl2 | 382 (M + H)+ [FAB] | B4a, C4a |
| 310 | Me | 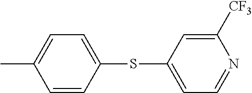 | | 0.32 | 70% acetone/ 30% CH2Cl2 | 450 (M + H)+ [FAB] | B3b, C4a |
| 311 | Me | 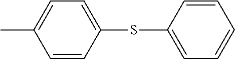 | | 0.09 | 50% EtOAc/ 50% hexane | 381 (M + H)+ [FAB] | C4a |
| 312 | Me | 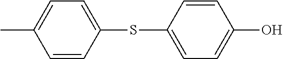 | | 0.61 | 100% EtOAc | 397 (M + H)+ [FAB] | B3c, C4a |
| 313 | Me | 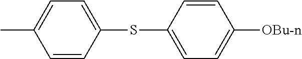 | | 0.25 | 50% EtOAc/ 50% hexane | 453 (M + H)+ [FAB] | B5, C4a |
| 314 | Me | 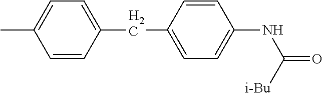 | | 0.65 | 100% EtOAc | 462 (M + H)+ [FAB] | B6, C4a |
| 315 | Me | 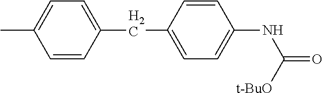 | | 0.67 | 100% EtOAc | 478 (M + H)+ [FAB] | B6, C4a |
| 316 | Me | 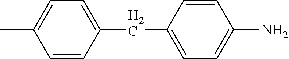 | | 0.50 | 100% EtOAc | 378 (M + H)+ [FAB] | C4a |
| 317 | Me | 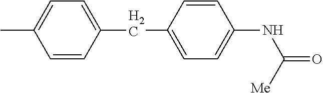 | | 0.33 | 100% EtOAc | 420 (M + H)+ [FAB] | C4a, D3 |
| 318 | Me | 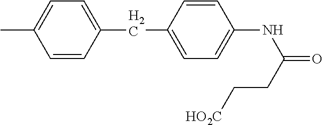 | | 0.60 | 10% water/ 90% CH3CN | 478 (M + H)+ [FAB] | C4a, D3 |
| 319 | Me | 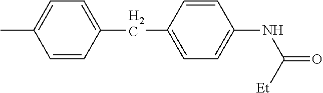 | | 0.55 | 100% EtOAc | 434 (M + H)+ [FAB] | C4a, D3 |
| 320 | Me | 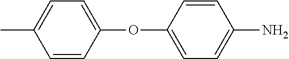 | | 0.52 | 100% EtOAc | 380 (M + H)+ [FAB] | C4a |
| 321 | Me | 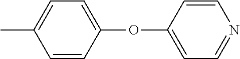 | | 0.25 | 60% acetone/ 40% CH2Cl2 | 366 (M + H)+ [FAB] | C4a |
| 322 | Me | 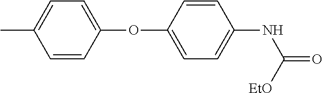 | | 0.52 | 100% EtOAc | 452 (M + H)+ [FAB] | C4a, D3 |
| 323 | Me | 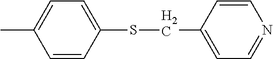 | | 0.34 | 60% acetone/ 40% CH2Cl2 | 396 (M + H)+ [FAB] | C4a |
| 324 | Me | 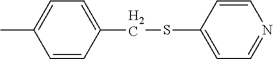 | | 0.36 | 60% acetone/ 40% CH2Cl2 | 396 (M + H)+ [FAB] | C4a |
| 325 | Me | 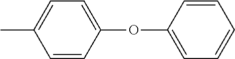 | 147- 149 | | | 365 (M + H)+ [FAB] | C1c |
| 326 | Me | 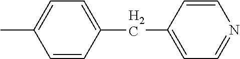 | 161- 162 | 0.15 | 4% MeOH/ 96% CH2Cl2 | 364 (M + H)+ [FAB] | C2b |
| 327 | Me | 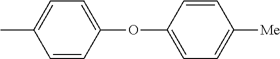 | 228 dec | | | 379 (M + H)+ [FAB] | C2b |
| 328 | Me | 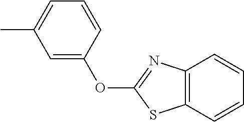 | | 0.30 | 5% MeOH/ 95% CH2Cl2 | 422 (M + H)+ [FAB] | C2b |
| 329 | Me | 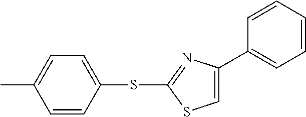 | | 0.46 | 100% EtOAc | 464 (M + H)+ [FAB] | B3c, C4a |
| 330 | Me | 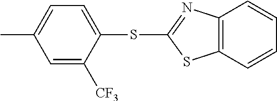 | | 0.52 | 100% EtOAc | 506 (M + H)+ [FAB] | B3c, C4a |
| 331 | Me | 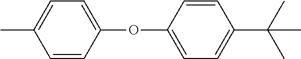 | | 0.75 | 100% EtOAc | 421 (M + H)+ [FAB] | B3c, C4a |
| 332 | Me | 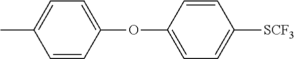 | | 0.50 | 100% EtOAc | 465 (M + H)+ [FAB] | B3c, C4a |
| 333 | Me | 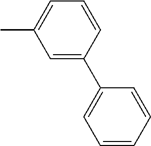 | | 0.50 | 100% EtOAc | 349 (M + H)+ [FAB] | C4a |
| 334 | Me | 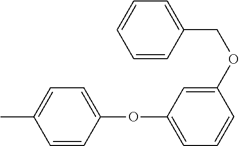 | | 0.60 | 100% EtOAc | 471 (M + H)+ [FAB] | B2, C4a |
| 335 | Me | 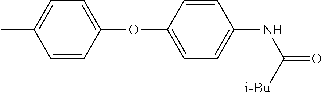 | | 0.52 | 100% EtOAc | 466 (M + H)+ [FAB] | C4a, D3 |
| 336 | Me | 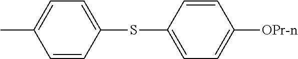 | | 0.42 | 100% EtOAc | 439 (M + H)+ [FAB] | B5, C4a |
| 337 | —CH₂—CF₃ | 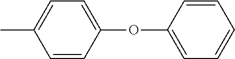 | | | | 433 (M + H)+ [FAB] | C3a |
| 338 | —(CH₂)₂CN | 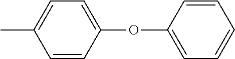 | | 0.37 | 50% EtOAc/ 50% hexane | 404 (M + H)+ [HPLC ES-MS] | A3, C1b |
| 339 | 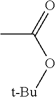 | 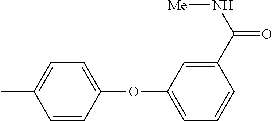 | 159- 161 | | | 508 (M + H)+ [FAB] | A5, B6, C2b |

TABLE 4
5-Substituted-2-thiadiazolyl Ureas

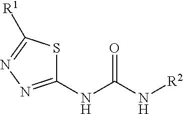

| | | | mp | TLC | Solvent | Mass Spec. | Synth. |
|---|---|---|---|---|---|---|---|
| Entry | R¹ | R² | (° C.) | R_{f} | System | [Source] | Method |
| 340 | t-Bu | 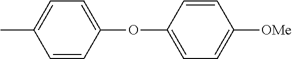 | | 0.37 | 5% MeOH/ 95% CH2Cl2 | 399 (M + H)+ [FAB] | B3a, C3a |
| 341 | t-Bu | 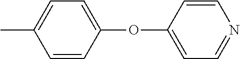 | | 0.26 | 5% MeOH/ 95% CH2Cl2 | 370 (M + H)+ [FAB] | C3a |
| 342 | t-Bu | 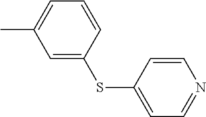 | | | | 386 (M + H)+ [FAB] | B4a, C3a |
| 343 | t-Bu | 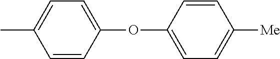 | | 0.30 | 5% acetone/ 95% CH2Cl2 | 383 (M + H)+ [FAB] | C1b |
| 344 | t-Bu | 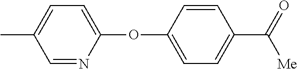 | | 0.60 | 10% MeOH/ CH2Cl2 | 412 (M + H)+ [FAB] | C3b |
| 345 | t-Bu | 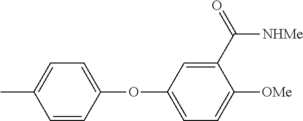 | 245- 250 | 0.23 | 100% EtOAc | 456 (M + H)+ [HPLC ES-MS] | B3a step 1, B12, D5b step 2, C3a |
| 346 | t-Bu | 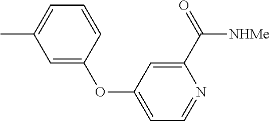 | | 0.10 | 50% EtOAc/ 50% pet ether | | C3b |
| 347 | 5-Bu | 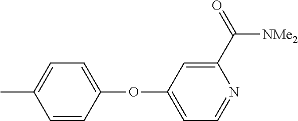 | | 0.13 | 50% EtOAc/ 50% pet ether | 441 (M + H)+ [HPLC ES-MS] | C3b |
| 348 | t-Bu | 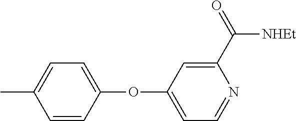 | | 0.14 | 5% MeOH/ 45% EtOAc/ 50% hexane | 441 (M + H)+ [HPLC ES-MS] | C3b, D5b |
| 349 | t-Bu | 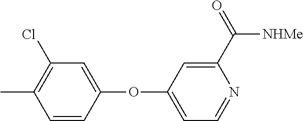 | | 0.23 | 5% MeOH/ 45% EtOAc/ 50% hexane | 461 (M + H)+ [HPLC ES-MS] | C3b, D5b |
| 350 | t-Bu | 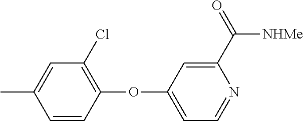 | | 0.09 | 5% MeOH/ 45% EtOAc/ 50% hexane | 461 (M + H)+ [HPLC ES-MS] | C3b, D5b |
| 351 | t-Bu | 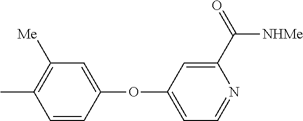 | | 0.13 | 5% MeOH/ 45% EtOAc/ 50% hexane | 441 (M + H)+ [HPLC ES-MS] | C3b, D5b |
| 352 | t-Bu | 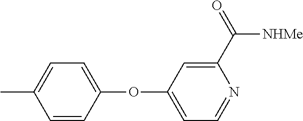 | 159- 160 | 0.10 | 50% EtOAc/ 50% pet ether | 427 (M + H)+ [HPLC ES-MS] | C3b |
| 353 | t-Bu | 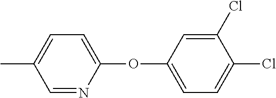 | | 0.47 | 10% MeOH/ CH2Cl2 | 438 (M + H)+ [FAB] | C3b |
| 354 | t-Bu | 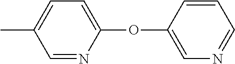 | | 0.31 | 10% MeOH/ CH2Cl2 | 371 (M + H)+ [FAB] | C3b |
| 355 | t-Bu | 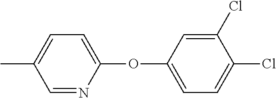 | | 0.51 | 10% MeOH/ CH2Cl2 | 400 (M + H)+ [FAB] | C3b |
| 356 | t-Bu | 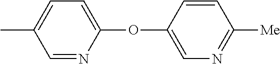 | | 0.43 | 10% MeOH/ CH2Cl2 | 385 (M + H)+ [FAB] | C3b |
| 357 | t-Bu | 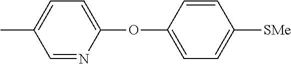 | | 0.70 | 10% MeOH/ CH2Cl2 | 416 (M + H)+ [FAB] | C3b |
| 358 | t-Bu | 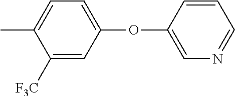 | | 0.11 | 50% EtOAc/ 50% hexane | 438 (M + H)+ [HPLC ES-MS] | C8 |
| 359 | t-Bu | 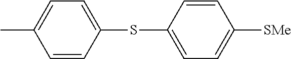 | | 0.06 | 5% MeOH/ 95% CH2Cl2 | 432 (M + H)+ [FAB] | C3b |
| 360 | t-Bu | 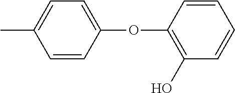 | | 0.20 | 50% EtOAc/ 50% hexane | 385 (M + H)+ [HPLC ES-MS] | C8 |
| 361 | t-Bu | 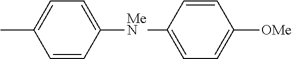 | 107- 110 | 0.05 | 30% EtOAc/ 70% hexane | 412 (M + H)+ [HPLC ES-MS] | C3a |
| 362 | t-Bu | 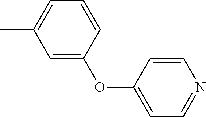 | | 0.16 | 100% EtOAc | 370 (M + H)+ [HPLC ES-MS] | C8 |
| 363 | 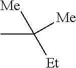 | 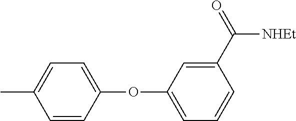 | | 0.12 | 100% EtOAc | | C4a, D5b |
| 364 | 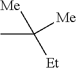 | 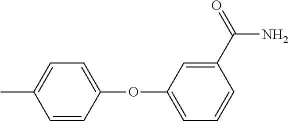 | 183- 185 | | | | B3d step 2, C3a |
| 365 | 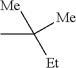 | 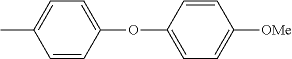 | | 0.19 | 6% MeOH/ 94% CHCl3 | 413 (M + H)+ [FAB] | A6, C3b |
| 366 | 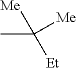 | 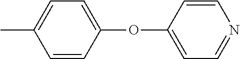 | 248- 249 | 0.34 | 6% MeOH/ 94% CHCl3 | | A6, C3b |
| 367 | 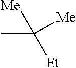 | 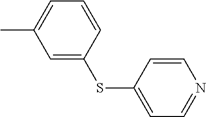 | | 0.20 | | 400 (M + H)+ [FAB] | A6, C3b |
| 368 | 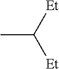 | 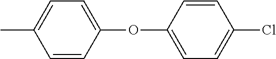 | 182- 183 | 0.33 | 5% MeOH/ 95% CHCl3 | | A6, C3b |
| 369 | 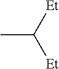 | 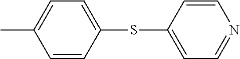 | 180- 181 | 0.19 | 5% MeOH/ 95% CHCl3 | | A6, C3b |
| 370 | 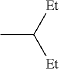 | 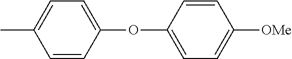 | 168- 169 | 0.24 | 5% MeOH/ 95% CHCl3 | | A6, C3b |
| 371 | 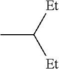 | 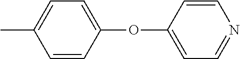 | 168- 171 | 0.17 | 6% MeOH/ 94% CHCl3 | | A6, C3b |
| 372 | 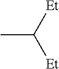 | 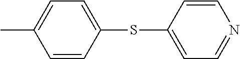 | 156- 158 | 0.19 | 6% MeOH/ 94% CHCl3 | | A6, C3b |

TABLE 5
5-Substituted-3-thienyl Ureas

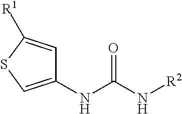

| | | | mp | TLC | Solvent | Mass | Synth. |
|---|---|---|---|---|---|---|---|
| Entry | R¹ | R² | (° C.) | R_{f} | System | Spec. | Method |
| 373 | t-Bu | 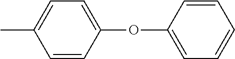 | 144- 145 | 0.68 | 5% acetone/ 95% CH2Cl2 | | A4b, C1a |
| 374 | t-Bu | 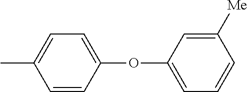 | | 0.52 | 30% Et2O/ 70% pet ether | 381 (M + H)+ (HPLC ES-MS] | |
| 375 | t-Bu | 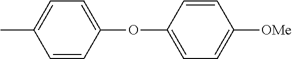 | | 0.26 | 30% Et2O/ 70% pet ether | 397 (M + H)+ [HPLC ES-MS] | need recipie |
| 376 | t-Bu | 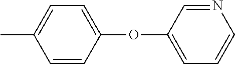 | | 0.28 | 50% Et2O/ 50% pet ether | 368 (M + H)+ [HPLC ES-MS] | need recipie |
| 377 | t-Bu | 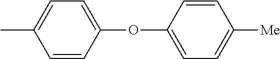 | 57 | | | 381 (M + H)+ [FAB] | A4a |
| 378 | t-Bu | 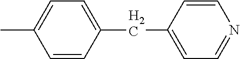 | | 0.15 | 50% EtOAc/ 50% pet ether | 365 (M+) [EI] | A4a |
| 379 | t-Bu | 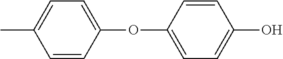 | | 0.44 | 50% EtOAc/ 50% pet ether | 383 (M + H)+ [FAB] | A4a |
| 380 | t-Bu | 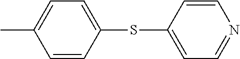 | | | | 384 (M + H)+ [FAB] | A4a |
| 381 | t-Bu | 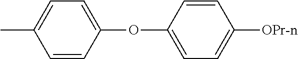 | 176- 177 | 0.45 | 20% EtOAc/ 80% hexane | 425 (M + H)+ [FAB] | D2 |

TABLE 5
Additional Ureas

| | | mp | TLC | Solvent | Mass Spec. | Synth. |
|---|---|---|---|---|---|---|
| Entry | R² | (° C.) | R_{f} | System | [Source] | Method |
| 382 | 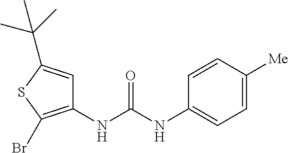 | 161- 163 | 0.71 | 20% EtOAc/ 80% hexane | 367 (M + H)+, 369 (M + 3)+ [FAB] | D1 |
| 383 | 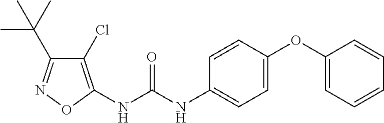 | 145- 147 | 0.57 | 5% MeOH/ 95% CHCl3 | | A2, C3b |
| 384 | 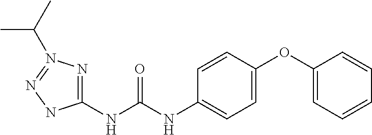 | 132- 135 | 0.33 | 5% acetone/ 95% CH2Cl2 | 339 (M + H)+ (HPLC ES- MS] | A9, C1d |
| 385 | 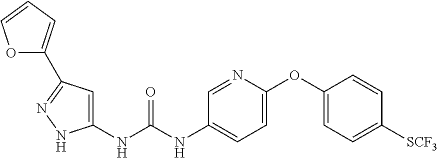 | | 0.60 | 50% EtOAc/ 50% hexane | 462 (M + H)+ [HPLC ES- MS] | C8 |
| 386 | 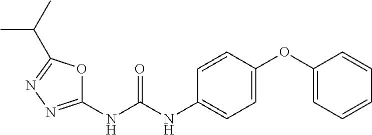 | | 0.28 | 5% acetone/ 95% CH2Cl2 | 339 (M + H)+ [FAB] | A7, C1d |
| 387 | 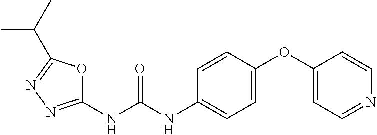 | | | | 3440 (M + H)+ [FAB] | B3b step 1,2, C1d |
| 388 | 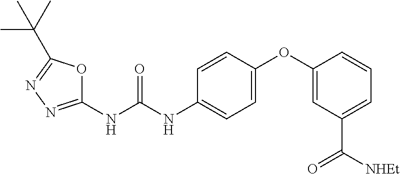 | 174-5 | | | 424 (M + H)+ [HPLC ES- MS] | B4b, C8 |
| 389 | 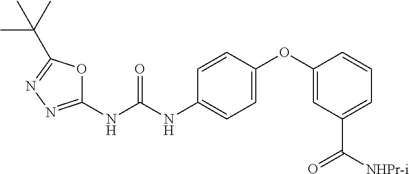 | 198- 200 | | | | C3b, D5b |
| 390 | 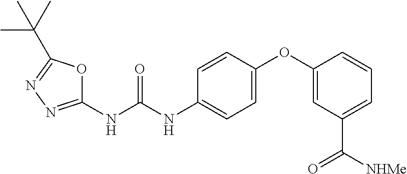 | 169- 170 | 0.23 | 100% EtOAc | | B4b, C8 |
| 391 | 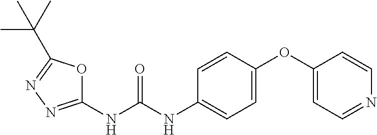 | 167- 171 | 0.12 | 100% EtOAc | | B4b, C8 |
| 392 | 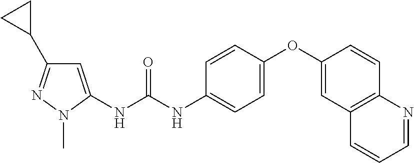 | | 0.08 | 50% EtOAc/ 50% hexane | 400 (M + H)+ [HPLC ES- MS] | C8 |
| 393 | 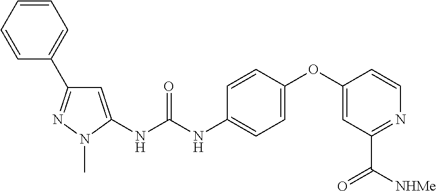 | | 0.55 | 90% EtOAc/ 10% hexane | 443 (M + H)+ [FAB] | B10, B4b, C2b |
| 394 | 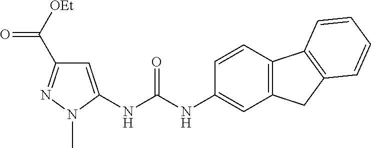 | 230 dec | | | 377 (M + H)+ [HPLC ES- MS] | C5 |
| 395 | 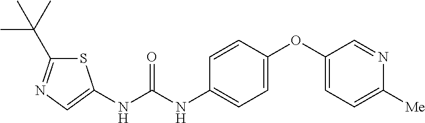 | | 0.48 | 50% EtOAc/ 50% hexane | 383 (M + H)+ [FAB] | C8 |
| 396 | 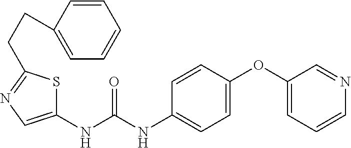 | | | | 417 (M + H)+ [HPLC ES- MS] | C8 |
| 397 | 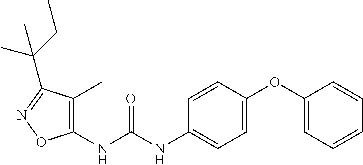 | 155- 157 | 0.44 | 5% acetone/ 95% CH2Cl2 | 380 (M + H)+ [FAB] | C1b |

### Biological Examples

In Vitro raf Kinase Assay:

In an in vitro kinase assay, raf is incubated with MEK in 20 mM Tris-HCl, pH 8.2 containing 2 mM 2-mercaptoethanol and 100 mM NaCl. This protein solution (20 μL) is mixed with water (5 μL) or with compounds diluted with distilled water from 10 mM stock solutions of compounds dissolved in DMSO. The kinase reaction is initiated by adding 25 μL [γ-³³P]ATP (1000-3000 dpmm/pmol) in 80 mM Tris-HCl, pH 7.5, 120 mM NaCl, 1.6 mM DTT, 16 mM MgCl₂. The reaction mixtures are incubated at 32° C., usually for 22 min. Incorporation of ³³P into protein is assayed by harvesting the reaction onto phosphocellulose mats, washing away free counts with a 1% phosphoric acid solution and quantitating phosphorylation by liquid scintillation counting. For high throughput screening, 10 μM ATP and 0.4 μM MEK are used. In some experiments, the kinase reaction is stopped by adding an equal amount of Laemmli sample buffer. Samples are boiled 3 min and the proteins resolved by electrophoresis on 7.5% Laemmli gels. Gels are fixed, dried and exposed to an imaging plate (Fuji). Phosphorylation is analyzed using a Fujix Bio-Imaging Analyzer System.

All compounds exemplified displayed IC₅₀s of between 1 nM and 10 μM.

### Cellular Assay:

For in vitro growth assay, human tumor cell lines, including but not limited to HCT116 and DLD-1, containing mutated K-ras genes are used in standard proliferation assays for anchorage dependent growth on plastic or anchorage independent growth in soft agar. Human tumor cell lines were obtained from ATCC (Rockville Md.) and maintained in RPMI with 10% heat inactivated fetal bovine serum and 200 mM glutamine. Cell culture media and additives are obtained from Gibco/BRL (Gaithersburg, Md.) except for fetal bovine serum (JRH Biosciences, Lenexa, Kans.). In a standard proliferation assay for anchorage dependent growth, 3×10³ cells are seeded into 96-well tissue culture plates and allowed to attach overnight at 37° C. in a 5% CO₂ incubator. Compounds are titrated in media in dilution series and added to 96 well cell cultures. Cells are allowed to grow 5 days typically with a feeding of fresh compound containing media on day three. Proliferation is monitored by measuring metabolic activity with standard XTT calorimetric assay (Boehringer Mannheim) measured by standard ELISA plate reader at OD 490/560, or by measuring ³H-thymidine incorporation into DNA following an 8 h culture with 1 μCu ³H-thymidine, harvesting the cells onto glass fiber mats using a cell harvester and measuring ¹H-thymidine incorporation by liquid scintillant counting.

For anchorage independent cell growth, cells are plated at 1×10³ to 3×10³ in 0.4% Seaplaque agarose in RPMI complete media, overlaying a bottom layer containing only 0.64% agar in RPMI complete media in 24-well tissue culture plates. Complete media plus dilution series of compounds are added to wells and incubated at 37° C. in a 5% CO, incubator for 10-14 days with repeated feedings of fresh media containing compound at 3-4 day intervals. Colony formation is monitored and total cell mass, average colony size and number of colonies are quantitated using image capture technology and image analysis software (Image Pro Plus, media Cybernetics). These assays establish that the compounds of Formula I are active to inhibit raf kinase activity and to inhibit oncogenic cell growth.

### In Viva Assay:

An in vivo assay of the inhibitory effect of the compounds on tumors (e.g., solid cancers) mediated by raf kinase can be performed as follows:

CDI nu/nu mice (6-8 weeks old) are injected subcutaneously into the flank at 1×10⁶ cells with human colon adenocarcinoma cell line. The mice are dosed i.p., i.v. or p.o. at 10, 30, 100, or 300 mg/Kg beginning on approximately day 10, when tumor size is between 50-100 mg. Animals are dosed for 14 consecutive days once a day; tumor size was monitored with calipers twice a week.

The inhibitory effect of the compounds on raf kinase and therefore on tumors (e.g., solid cancers) mediated by raf kinase can further be demonstrated in vivo according to the technique of Monia et al. (Nat. Med. 1996, 2, 668-75).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1-77. (canceled)

78. A compound of the following formula 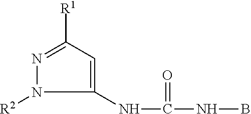
wherein R² is selected from the group consisting of H, —C(O)R⁴, —CO₂R⁴, —C(O)NR³R^{3′}, C₁-C₁₀ alkyl, C₃-C₁₀ cycloalkyl, C₇-C₂₄ alkaryl, C₄-C₂₃ alkheteroaryl, substituted C₁-C₁₀ alkyl, substituted C₃-C₁₀ cycloalkyl, substituted C₇-C₂₄ alkaryl and substituted C₄-C₂₃ alkheteroaryl, where if R² is a substituted group, it is substituted by one or more substituents independently selected from the group consisting of —CN, —CO₂R⁴, —C(O)—NR³R^{3′}, —NO₂, —OR⁴, —SR⁴, and halogen up to per-halosubstitution,
wherein R³ and R^{3′} are independently selected from the group consisting of H, —OR⁴, —SR⁴, —NR⁴R^{4′}, —C(O)R⁴, —CO₂R⁴, —C(O)NR⁴R^{4′}, C₁-C₁₀ alkyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl; and
wherein R⁴ and R^{4′} are independently selected from the group consisting of H, C₁-C₁₀ alkyl, C₃-C₁₀ cycloalkyl, C₆-C₁₄ aryl, C₃-C₁₃ heteroaryl; C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl,
wherein R^{I} is selected from the group consisting of C₃-C₁₀ alkyl, C₃-C₁₀ cycloalkyl, up to per-halosubstituted C₁-C₁₀ alkyl and up to per-halosubstituted C₃-C₁₀ cycloalkyl;
B is
-Q-(Y-Q¹)ₛ-Zₙ₁
wherein
Y is —O—, —S—, —CH₂S—, —SCH₂—, —CH₂O—, —OCH₂— or —CH₂—,
Q is phenyl or pyridinyl, substituted or unsubstituted by halogen, up to per-halosubstitution;
Q¹ is pyridinyl, phenyl or benzothiazolyl, unsubstituted or unsubstituted by halogen up to per-halosubstitution,
Z is —SCH₃, or —NH—C(O)—CₚH₂ₚ₋₁,
p is 1-4,
s=1, and
n1=0-1,
or a pharmaceutically acceptable salt thereof.

79. A pharmaceutical composition comprising a compound of claim 78 and a pharmaceutically acceptable carrier.

80. A compound of claim 78, wherein Q is phenyl substituted or unsubstituted by halogen, up to per-halosubstitution.

81. A compound of claim 78, wherein Q is pyridinyl substituted or unsubstituted by halogen, up to per-halosubstitution.

82. A compound of claim 80, wherein Q¹ is pyridinyl, unsubstituted or unsubstituted by halogen up to per-halosubstitution.

83. A compound of claim 80, wherein Q¹ is phenyl, unsubstituted or unsubstituted by halogen up to per-halosubstitution.

84. A compound of claim 80, wherein Q¹ is benzothiazolyl, unsubstituted or unsubstituted by halogen up to per-halosubstitution.

85. A compound of claim 81, wherein Q¹ is pyridinyl, unsubstituted or unsubstituted by halogen up to per-halosubstitution.

86. A compound of claim 81, wherein Q¹ is phenyl, unsubstituted or unsubstituted by halogen up to per-halosubstitution.

87. A compound of claim 81, wherein Q¹ is benzothiazolyl, unsubstituted or unsubstituted by halogen up to per-halosubstitution.

88. A method for the treatment of cancerous cell growth comprising administering an effective amount of a compound of claim 78 to a patient in need thereof.

89. A method according to claim 88, wherein the cancerous cell growth is mediated by raf kinase.

90. A method according to claim 88, wherein lung carcinoma is treated.

91. A method according to claim 88, wherein pancreas carcinoma is treated.

92. A method according to claim 88, wherein thyroid carcinoma is treated.

93. A method according to claim 88, wherein bladder carcinoma is treated.

94. A method according to claim 88, wherein colon carcinoma is treated.

95. A method according to claim 88, wherein myeloid leukemia is treated.
